SEQUENCE LISTING (7 sequences)

SEQ ID NO: 1 - protein, 468 aa, artificial sequence
  synthetic polypeptide
MAVLGLLFCLVTFPSCVLSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 234 aa, artificial sequence
  synthetic polypeptide
MVSTPQFLVFLLFWIPASRGDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - DNA, 1428 bp, artificial sequence
  synthetic polynucleotide
ctcgagaccgccatggccgtgctgggcctgctgttctgcctggtgaccttccccagctgcgtgctgtcccaggtgcagctgaagcagagcggccctggcctggtgcagccctctcagtccctctccattacctgtaccgtgtccggcttctctctcacaaattatggagtccattgggtgcgccagagccccggcaagggcctggagtggctgggcgtgatctggtccggcggcaacaccgactacaacacccccttcaccagcagactgagcatcaacaaggacaacagcaagagccaggtgttcttcaagatgaacagcctgcagagcaacgacaccgccatctactactgcgctcgcgctctcacctactacgactacgagttcgcctactggggccagggcaccctggtgaccgtgtccgccgccagcaccaagggccccagcgtgttccccctggcccccagcagcaagagcaccagcggcggcaccgctgccctgggctgtctggtgaaggactacttccccgagcccgtgaccgtgtcttggaactctggcgccctgacctccggcgtgcacacatttccagctgtcctccagagttccggactgtattccctcagttctgtcgtgacagtcccttcctctagcctgggcacccagacctacatctgcaacgtgaaccacaagcccagcaacaccaaggtggacaagagagtggagcccaagagctgcgacaagacccacacctgcccaccatgtcctgctccagagctgctggggggaccctccgtgttcctgttcccccccaagcccaaggacaccctgatgatcagcaggacccccgaggtgacctgcgtggtggtggacgtgtcccacgaggaccctgaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctgccagcccccatcgaaaagaccatcagcaaggccaagggccagcctcgcgagccccaggtgtacaccctgcccccctcccgcgaggagatgaccaagaaccaggtgtccctgacctgcctggtgaagggcttctaccccagcgatatcgccgtggagtgggagagcaacggccagcccgagaacaactacaagaccaccccccctgtgctggacagcgacggcagcttcttcctgtacagcaagctgaccgtggacaagtccaggtggcagcagggaaatgtcttttcttgttctgtcatgcatgaagctctgcacaaccactacacccagaagtccctgagcctgtcccccggcaagtgatagctcgag

SEQ ID NO: 4 - DNA, 726 bp, artificial sequence
  synthetic polynucleotide
ctcgagaccgccatggtgtccaccccccagttcctcgtgttcctgctgttctggatccccgccagcaggggcgacatcctgctgacccagagccccgtgatcctgagcgtgtctcccggcgagagggtgtccttcagctgcagagccagccagagcatcggcaccaacatccactggtatcagcagaggaccaacggcagccccaggctgctgatcaagtacgccagcgagtccatcagcggcatccccagcaggttcagcggcagcggctccggcaccgacttcaccctgagcatcaacagcgtggagagcgaggatatcgccgactactactgccagcagaacaacaactggcccaccaccttcggcgctggcaccaagctggagctgaagaggaccgtggccgctcccagcgtgttcatcttcccccccagcgacgagcagctgaagtccggcaccgccagcgtggtgtgcctgctgaacaacttctacccccgcgaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcacaaggtgtacgcctgcgaggtgacccaccagggcctgtccagccccgtgaccaagagcttcaacaggggcgagtgctgatagctcgag

SEQ ID NO: 5 - DNA, 24342 bp, artificial sequence
  synthetic polynucleotide
gtcgactctagagggacagcccccccccaaagcccccagggatgtaattacgtccctcccccgctaggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaagctttaggctgaaagagagatttagaatgacagaatcatagaacggcctgggttgcaaaggagcacagtgctcatccagatccaaccccctgctatgtgcagggtcatcaaccagcagcccaggctgcccagagccacatccagcctggccttgaatgcctgcagggatggggcatccacagcctccttgggcaacctgttcagtgcgtcaccaccctctgggggaaaaactgcctcctcatatccaacccaaacctcccctgtctcagtgtaaagccattcccccttgtcctatcaagggggagtttgctgtgacattgttggtctggggtgacacatgtttgccaattcagtgcatcacggagaggcagatcttggggataaggaagtgcaggacagcatggacgtgggacatgcaggtgttgagggctctgggacactctccaagtcacagcgttcagaacagccttaaggataagaagataggatagaaggacaaagagcaagttaaaacccagcatggagaggagcacaaaaaggccacagacactgctggtccctgtgtctgagcctgcatgtttgatggtgtctggatgcaagcagaaggggtggaagagcttgcctggagagatacagctgggtcagtaggactgggacaggcagctggagaattgccatgtagatgttcatacaatcgtcaaatcatgaaggctggaaagcctccaagatccccaagaccaaccccaacccacccaccgtgcccactggccatgtccctcagtgccacatccccacagttcttcatcacctccagggacggtgacccccccacctccgtgggcagctgtgccactgcagcaccgctctttggagaaggtaaatcttgctaaatccagcccgaccctcccctggcacaacgtaaggccattatctctcatccaactccaggacggagtcagtgaggatggggctctagagggacagcccccccccaaagcccccagggatgtaattacgtccctcccccgctaggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaagctttaggctgaaagagagatttagaatgacagaatcatagaacggcctgggttgcaaaggagcacagtgctcatccagatccaaccccctgctatgtgcagggtcatcaaccagcagcccaggctgcccagagccacatccagcctggccttgaatgcctgcagggatggggcatccacagcctccttgggcaacctgttcagtgcgtcaccaccctctgggggaaaaactgcctcctcatatccaacccaaacctcccctgtctcagtgtaaagccattcccccttgtcctatcaagggggagtttgctgtgacattgttggtctggggtgacacatgtttgccaattcagtgcatcacggagaggcagatcttggggataaggaagtgcaggacagcatggacgtgggacatgcaggtgttgagggctctgggacactctccaagtcacagcgttcagaacagccttaaggataagaagataggatagaaggacaaagagcaagttaaaacccagcatggagaggagcacaaaaaggccacagacactgctggtccctgtgtctgagcctgcatgtttgatggtgtctggatgcaagcagaaggggtggaagagcttgcctggagagatacagctgggtcagtaggactgggacaggcagctggagaattgccatgtagatgttcatacaatcgtcaaatcatgaaggctggaaagcctccaagatccccaagaccaaccccaacccacccaccgtgcccactggccatgtccctcagtgccacatccccacagttcttcatcacctccagggacggtgacccccccacctccgtgggcagctgtgccactgcagcaccgctctttggagaaggtaaatcttgctaaatccagcccgaccctcccctggcacaacgtaaggccattatctctcatccaactccaggaacggagtcagtgaggatggggctctagaggatccctcgacctgcaggtcaacggatcacaacaaactggaaaattcttcaagagaagaataccagaccaccctacctgcttcctgagaaatctgtttgctgctcagaagcaacagttagaaccagacatggaacaacagactggttccaaatcaggaaaggagtatgtcaaggctgtatatcgtcaccctgattatttaacttatatgcatagtacataatacaaaatgccaggctggatgaatcgcaagctggaatcaagatttctgggagaaatatcaataaacgagatacaaagatacaccacacttatggcagaaaactaagaagaactaaagagcctcttgatgaaagtgaaagaggagagtgaaaaagccagcttaaaacccaacattcaaaatcaagatcatcatttcatggcaaataaatggggaaacaatggaaacagtgagagactttattttcttgggctccaaaatcactgcagattgtgactacagccatgattaaaagatgcttgctccttggaagagaagctattaccaaactagaaagcatattaaaaagcagagacgttactttgctgactaagttctgtctagtcaaacctatggtttttccagtagtcatatatggatgtgagttgaactataaagaaagctgagcaccaaagaattgatgcttttgaaatttggtgttggagaagtctcttgagagtcccttgaacctgcaaggagatccaaccagtccatcctaaaggaaatcagtcctgaatattcattggaaggactgatgctgaaattgaagattaacgttttggactcacctaatgcagaagagccaactcactagaaaagaccccatgttggcaaaaattgaagccaggaagagaagtgaatgacagaggatgagatggttggatggcatcgttgactgaatggacatgagtctgatcaagttccgggagacagcaaaggacagggctgcctggtctgctgcagtccatggggttgcaaagagtcggtctcaaatgagtaactaaacaacaaccaagcagtagaaaaataaataaaatttgtctctgagatctcagtacctctttctgtgcatatccgtctcctgttattgtactttgtcttctgcttgtaataaagctgtcctgttagtaaaatctgtttgggtcctctgaattcttttagctatcaaaaatggaaggtgattattgtgcaatgtccacctctgagtaatatacagagaataaaagaagggagaaattatgtgcaagttctctctcatctcctgcttctcatttaaaagattctacctcagtgggggctaaaactccacatttaacagtagcaaaaaccaatattccatagcttcttaggaaaccattttttatactcttgtatgtaattacattcaagctcaaaagcaaagaagtgattctgcgttggtgaaggcccaaccatagaaaagaggaagaaaataggccacatactgtgcttcccccatagctcagttggtaaagaatctacctacaatgcaggaggcctgggcttgatccctgggtaagggagatcccctggagaaggaaatggtaacccactccagtactcttgcctgtaaatcccatggacggaggagcctggcagctacagccttggggtggcaagagttggacatgattaacaactaaaccactgccaccactccacatactgagtgctccccagtggcactagtggtaaagaaccacctgccggtgcagaagacattaaagacactggctctatccctgcttgggaagtagggaagatcccctagagagggaaatagcaacccactccagaattcttgcctggaaaatcccatgaatgaagactggcgggctgtagtaactggggtcacaaagagttaaacatgatttagcaactaaacatcaccacattaaaaaaattaccaccaaaatagtcatattccaggctaaggggaataatagcactagtacctgagagaactttctcagattctctgtcaagttcttccttctctcatataaccagtagtctagtttacctcatcagatattaactactcatcgattctaaattatctaattatgggggggggcactacattgcattatattttgtgtccattgactatcactcaatttatttataaaaaattcatccatgttgtttctgtgacagtaactcattcacattaattgtaatatctcattgcattgtatactacaatttatttatacaaaatactattattcacacttctgttgattttaatttggaacatcaacaataacgtggctgagaagcttctttctttagtatattgttaaggatttccttgatcaagattttacctacttttctggtccaattggtgagagacagtcataaggaaatgctgtgtttattgcacaatatgtaaagcatcttcctgagaaaataaaagggaaatgttgaatgggaaggatatgctttcttttgtattccttttctgagaaatcagactttttcaccttggccttggccaccaaaagctaacaaataaaggcatatgaagtagccaaggccttttctagttatatctatgacactgagttcatttcatcatttattttcctgacttcctcctgggtccatatgagcagtcttagaatgaatattagctgaataatccaaatacatagtagatgttgatttgggttttctaagcaatccaagacttgtatgacagtaagatgtattaccatccaacacacatctcagcatgatataaatgcaaggtatattgtgaagaaaaatttttaattatgtcaaagtgcttactttagaaggtcatctatctgtcccaaagctgtgaatatatatattgaaggtaatgaatagatgaagctaaccttgtaaaaatgagtagtgtgaaatacaactacaattatgaacatctgtcactaaagaggcaaagaaacttgaagattgcttttgcaaatgggctcctattaataaaaagtacttttgaggtctggctcagactctattgtagtacttagggtaagaccctcctcctgtatgggctttcattttctttcttgcttccctcatttgcccttccatgaatactagctgataaacattgactataaaagatatgaggccaaacttgagctgtcccattttaataaatctgtataaataatatttgttctacaaaagtattatctaaataaatgttactttctgtcttaaaatccctcaacaaatccccactatctagagaataagattgacattccctggaatcacagcatgctttgtctgccattatctgacccctttctctttctctcttctcacctccatctactcctttttccttgcaattcatgacccagattcactgtttgatttggcttgcatgtgtgtgtgctgagttgcgtctgactgttatcaaccccatgaatgatagtccaccaggctctactgtccatgaaattttccagtcaagaatactggagtggattgcatttcctactccatttgattaatttagtgacttttaaatttctttttccatattcgggagcctattcttcctttttagtctatactctcttcactcttcaggtctaaggtatcatcgtgtgcttgttagcttgttactttctccattatagcttaagcactaacaactgttcaggttggcatgaaattgtgttctttgtgtggcctgtatatttctgttgtgtattagaatttaccccaagatctcaaagacccactgaatactaaagagacctcattgtggttacaataatttggggactgggccaaaacttccgtgcatcccagccaagatctgtagctactggacaatttcatttcctttatcagattgtgagttattcctgttaaaatgctccccagaatttctggggacagaaaaataggaagaattcaaatccactgttggttttatttcaaaccacaaaattagcatgccattaaatactatatataaacagccactaaatcagatcattatccattcagcttctccttcacttcttctcctctactttggaaaaaaggtaagaatctcagatataatttcagtgtatctgctactcatctttattttggactaggttaaaatgtagaaagaacataattgcttaaaatagatcttaaaaataagggtgtttaagataaggtttacactattttcagcagatatgttaaaaaatagaagtgactataaatacttgataaaaattatagtgactgcaaatgttttaggaatataataagatataataacagtggttgctattttctttagcacaagactagttaacaggctgtattaaaagatcttttcttgaattaaatattttcaatttgattaaacctacctcagccataaaggcaagcacatttcatttatactatggggatttgaataattattactgaagaagctctaccaacaaaaagtttatagagctatcatatttagtcaagagataaagagggttgttaggatatatatgctatttgaaaggtatttataaaagaagagtatatttatcaaaatttctcaagaacatccaaatttcaagtttatcatttatcttacaatatttcaaaaatattaaaatagatacatgaaatacagaagtaaattaaagagaaagtattttacttggtaaaaaaattctaggttggacagagagtgccaggaaacaaaaacaatgaaaaatgtgacctgacaggaattatagctcaaagtatagtagtaagtaatgaaatggcttaaaaattggtatataaaatgctagttataaaataaacaaaatgcaataatatcctccctacatgtaatgaattctaggtattatgattatgctcttttttgaagtcttgacaataaaaatttttttagaagtttataggcatcttgaataaagtgaaacaaattaagaattagtatccatgagaaaaatatagaacaattttcctaatttagtttgaaaatctgggattgaagatgtgtgtcaagagatgttggtggcaagaacatttttttttcaagaacttataaaaatgcaacaaaacaaaccatttaatacattttggtcaaaatcaataatgtattttattttatgctccaaggagcataaaattggggactgggcaagagaaactgacaccctggtaaattaccaagagataagtacacagttactatagtagaaaataagcatagtgtatgatctctaaaattatgtgagacaaaggagagatgacattaggcatgtggggatgaagactgagtagagaagaaacaatctaatcagtccaagaaaacatctcgatcagtggaacaaatagaagaaatgctaaaatgaaacagaagtcttactggaaataaaagatatgcataagacaaaaattcatgaaaatcacttagtttagcagagaaaagataaaaataaagtatgaccttcttcatatacattgtttgatcatatgcacctcaataaaactgagtctccaacagaaatgaaacattaatattttgttcactgctctaatcccagaatctaagcgatatctggcaataaaaataataaatatatattttttaataaatgaatcaaccacttaatttttctgtaaatatctgtaacttctcttctgtctttccaaaaacactcataagtactgtgaatgagatgaaaaagagtgaagtaggatataggctgttagcagaaaacatctgaatggctggcagtgaaacattaacttgaaatgtaagattaatgagtaatagtaaattttaaccttggccatatgataaaatgttcattaatatttttctagaatacagggctttttgtttttgccatgaggtttgcaggatcttggttccctgaccagggatcaaacctgcacaccagggatcaaacctgcactcccctggaagcatggagtcttggacatttgtattatacactatctttggttccttttaaagggaagtaattttacttaaataagaaaatagattgacaagtaatacgctgtttcctcatcttcccattcacaggaatcgcggatcctcgagaccgccatggtgtccaccccccagttcctcgtgttcctgctgttctggatccccgccagcaggggcgacatcctgctgacccagagccccgtgatcctgagcgtgtctcccggcgagagggtgtccttcagctgcagagccagccagagcatcggcaccaacatccactggtatcagcagaggaccaacggcagccccaggctgctgatcaagtacgccagcgagtccatcagcggcatccccagcaggttcagcggcagcggctccggcaccgacttcaccctgagcatcaacagcgtggagagcgaggatatcgccgactactactgccagcagaacaacaactggcccaccaccttcggcgctggcaccaagctggagctgaagaggaccgtggccgctcccagcgtgttcatcttcccccccagcgacgagcagctgaagtccggcaccgccagcgtggtgtgcctgctgaacaacttctacccccgcgaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcacaaggtgtacgcctgcgaggtgacccaccagggcctgtccagccccgtgaccaagagcttcaacaggggcgagtgctgatagctcgaggatccggacccttccctattcttgtaagtctaaatttactaactgtgctgtttaacttctgatgtttgtatgatatttgagtaattaagagccctacaaaaaaatcaataatgaatggttccaaaataagcatagctgagattaatgattctcagcattagttataaatagaataagctggaaaaccttcacctcccctccaccaccagatctcaatgtctaggcttacccatggagattctgattaactgttctttctatgtagaagaaacttattgggaagaaataatataatggactatgatttaattggtctgttgagaatttagatgaaggggattaagttacaataaagccagaatttaacttgataatctcatttggctaagaataacaaacctaagaaggtttgctattttctacaattttgaagttttccttatgcacaattatttcaccacatgactcatttcacatcttgtttttgatatatgagcatatgagggcaaaatactgaagatgcttatttcaatactcagggaaaattttcttgccaaaaggcaagaattgtataattcattcacttattttattttttttaatttttaaggtctaagaggatttcaaagtgaatgccccctcctcacttttggtaagctttaggagattggaggcagactgatcatttttatagttaatatcttttacatttcatcttcctggataagccccaatagtagcaatttctatcagtataccagcataaagattagttttaaatttattttcagtgattgactgttatttactgacctgaaattatgtatctgttatatttcaaataatgcaaaactgtatatatatggtgttgacagatttgattggttttctttcaattgcctatatccttattattgattgtaatcatttatagaaaaaacaaaataatttcttatacttttatgtaaacctgttagagcttattttaaagatcaactgcattcacatttctaatctagtcattatgagcttcaattgttttatctcacttaaaatttatatattgtcttttaattcatgagtcaaaatacaatctcacagtccagatatgggacttaaaaggggaatagcatatagttttgatattcttaaagatatacatctttttgtgatcatgattcagcagacattttaataaaacaattccaagtgagccgacacttggtcctagaggaatttttataaccttaagataaggcacagcatggtgtttttgtaataagatttcttttatgaaaaagtcacaccaaaattggaaatggggtgagatgaagagttataacatataactaaatggacatttgttctctattccacagaattgactgcgactggaaatatggcaacttttcaatccttgcatcatgctactaagataatttttaaatgagtatacatggaacaaaaaatgaaactttattcctttatttatattatgctttttcatcttaatttgaatttgagtcataaaccatatactttcaaaatgttaattcaacattagcataaaagttcaattttaacttggaaatatcatgaacatatcaaattatgtataaaaataatttctggaattgtgattattatttctttaagaatctatttcctaaccagtcatttcaataaattaacccttaggcatatttaagttttcttgtctttattatatttttaaaaatgaaattggtctctttattgttaacttaaatttatctttgatgttaaaaatagctgtggaaaattaaaattgaatagaattctttgaattgagttccaaaggatatcaaaaagtgagggaaaagatagggtgagcctatgctgcatatgtccttagaaagtcttggtttatacctgttacctaagttaaacaattatacttgttcctttcactctcgaaagtacccagcattggatgttaaattttatagtcatcctagacaaaaaaaaaaaaaaaaacaaacaaccctcaaatgtgatatctgaatcacagctctacagtgtggtagctaagtggtgctgtgtaagttagtctccaagagattccatttctacatttataaacagtcaatttaaggtgttttattgaagttttaatgtgaaaagtgcactatatggtgcatgataggagttcctggttgaatctcatttctgacatcactgacaccagtgcagcaaggactagtgttacaatcagaaggagctgagttgtgtaattttagccattaatgcccaagagactagaacttacacaaagctctaatatccattgtctctgtctgtggagtaattatttcattgccatgaattatctgtctgtcatatcctgcatttttatacatgattcagttcccttcagttcacacaatgacttgtctaatttcatctttcctgcatcctccatgttttcctcacttcaggattaagtgaagccgtacttaggcacaatatttcttatctttaaagaaaaattccatctttgagagttgttattgttcagtcactaggtcatgtccaactctttgtgaccccatgcactgcagcatgccaggcttccctgcccttcgctctctcctggagtttgctcagactcatgtagattgagtcggtgatggtatccaactatctcatcaactgttgtgcccttctcctcctaccctcagtctttaccagcatcagagtctttctcagattcttcaggttattatataacaactatcataaaaggagtatctaaatggctgtgtccattatttcacatgttattctctctttaacttgctccaatcccaattttatccctatgggaactgctttattgaagatcaccaacaacttttattttactaatcgttttgttttacccaacctctcagtgagtgttatgaggtagagttgactatttcttcattttgaaatattacgcttcatttcatttgatatcctaaagctcataaggtgtggtttttctcttaactcactagacacttctttgaagtctctcttctggcattttctccttttccaaaattttaatggttggagtaccctagattttagccttaatttgtttgatgttgttcagttccattctcagctcagagcttccaactgtatgtctccaaacttactcgttttgtaaactccaaactcatgcactcaactgcattcttgacctccacactgaattatctaattaatgtcctaaatctggcatgaccaagcatacatttttgtctgaatccagtccccaacttgctcaaaatttaattaaacgtaattcagttacaaaggcagctgatattgtatgcaatagacctgaatgggaacttcacaaaagaagttatcttaattgtcaataaaaacatgaaaaatactctacatcatcaatcttcagaaaaatgcaaattaaaggtgcctaataatatcatgacacaaccgtcagaatgactgaaatgaaaagaattgtaatacagttcagttcagttcagttactcagtcgtctccaactctttgtgaccccatgaactgcagcatgacagaccttcctgtccatcaccaactcccagagtttactcagactatgtccattgagttgatgatgccatccaaccatctcatcctctgtcgtccccttctcctcctgccctcagtctttcccagcatcagggtcttttccaatgagtcagctcttcgcatcaggtggctaaagtattggagtttcagcttcaacatcagtccttctaattaacacccaggactgatctcttttaggatggactagttggatctccttgcagtccaagggactctcaagagtcttctccaacaccacagttcaaaagcatcaattccttggcactcagctttccttatagtccatgtctcacatccacacatgactattggaaaaaccatagccttgactaggtggacctttgttgacaaagtaatgtctctgctttttaatatgttgtctagattggtcataactttccttccaagaagtaattgtcttttaatttcatggctgcagtcaccatctgcagtgattttggagccccaaaatataaagtcagctgctgtttccactgttgccccatctaccccatctatttgccatgaagtgatgggactggatgccactatcttagttttctgaatgttgagctttaagccagcctttttactctcctctttcactttcatcaagaggctctttagttcctcttcactttctgccataagggtggtgtcatctgcatatctgaggttattgatatttctcttggcaattttgattccagcctgcacttcttccagcccagtgtttctcatgatgtactctgcatataaattaaataagcagagtgacaatatacagccttgacatactctttttcctatttggaaccagtctgttgttccatgtccagttctaactgttgtttcctgacctgcatacaggtttctcaagaggcaagtcaggtggtctggtattctcacctgtttcagaattttccacagtttattgtgatccacacagtcaaaggctttggcatagccaataaagcagaaagagatgtttttctggaactctcttacttttttgatgatccagtggatgttggcaatttgatctctggttcctctgccttttctaaaaccagctttaacatctggaagttcatggttcacgtaatacaaaatgtaatacaaaatgtctgcaaaaacaaaggaatgaaaagtaatgctaaaaaatgttaatatttacagaaatttttatagtagtaaagaattcacctgcaatacaggagaaccgggttagatccctgggttggaagacctcctggagaaggaaatggctacccaatctagtattcttgtctggagaaggcaagaatggacagagaagcccagcgggctatggtccatcgggtcacaaagagtcagaagctaccttgcacacagcaagcacggtgcgcgcgcgtgcacacacacacacacacacacacacagacacacacacactctaaaacatttacccaagcttgtccaatggaaaatcaaaaagccagcaatttaagatgacatcaggtaccactgtccaggtaagcctcagaacacaatgaccagtaagaagcaaagtgccatatgagcaactcgaatttttgcaatgttacctaagagcttccatttttataatgcaaaagaatttcatatggggaaattgtattagataaccctgaatgaggagcaagatatagtcaaagtaagatgctctagtactattttttataagcatgatttgttcagccaaaggttttttcccatatggccaatgaactgaaatatgcagtcctgagatttgcatatatttctagctgaaaccaagtaaataatatcctcaagaaagaaatcaatagaaaagttggatgaagagtacaataaagggaccaaaaatattcagaaataagaactagaggagatattgggaaatccctggtgagtccagtttaggattttgtactttcactgcagttggcatggatataatccctcactggggaactaagatcccataagctgtgttggattgccaaaaaaataaatattaagagatatcattcatagaatattttaaagatattttagagaagaggaaattaaggatgtgagaatttgtattactttttcaagatactaaagctatttagagatagagctgttactaaaaacttcagtttcctaaaaattatttgaagcactgtttaataaattccaaaatatagaggaaggaaaacaaaatactgaggattcatataatgattcagatttagaaacaatataacacagaattagtgaattctgacaaattattaggtaggagtagatagttcagcattactcgtatagatggagtatttaatcctttccatgagattatccaaatataataatttcgtatctatgtgaagtataactattaagattactttataaagtaaatcaagaaccagagaataagaaaaatgttttgtgaaccagcagatactatgaacacataaaactcagaaccctgattcctaagacacacagctaatcctgattattcttcctttacatgtgaccatagaacttcacacaagttcaagatacatttgttgagcacatcagtatcagttcagtcactcagtcatgtccgaatctttgtgaccttgtggactgcagcacgccaggctttcctgtccaccaccaacccctggagcttactcaaactcatgtccattgagtcagtgatcccatccaaccatctcatcctctgtcatcctcttctcctgccttcaatctttcccagacattggagtcttttccaatgagtcagatcttcacattaggtggccaaagtataggagtttcagcttcagcatcaatccttccaatgaatattccttgatgtacccctttcgcagtttggaaccagtctgttgttccatgtccagttctaactgctgcttctggacctgtatacagatttctcaggaggcaggtaaagtggtctggtattcccatctcttgaagaattttccacagtttattgtgatccacacaatcaaaggctttagcgtagtcaataaagcagatgtttttctggaactctcgtgcttttttgatgatccaatggatgttggcaatttgatctctggttcctctgccttttctaaatccagcttgaacatctggaagttcatggtccacgtactgttgaagcctggcttggagaattttgagagttattttgctagcatgtgagatgagtgcaatcatgtgggtgtttgaacatactttgtcattgcttttctttgggattgtggcagtcctgtggccactgctgagttttccaaatttgctgacatattgagtgcagcactttcacagcatcaccttttaagatttgaaatagctcaactggaattccatcacctccactagctttgttcatagtgaggctttctaaggccgtttgactttgcattccagggtgtctggctctaggtgagtgatccgttgacctgcagcggccgagtcgactcggccgcgaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtcagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatctctagtcaaggcactatacatcaaatattccttattaacccctttacaaattaaaaagctaaaggtacacaatttttgagcatagttattaatagcagacactctatgcctgtgtggagtaagaaaaaacagtatgttatgattataactgttatgcctacttataaaggttacagaatatttttccataattttcttgtatagcagtgcagctttttcctttgtggtgtaaatagcaaagcaagcaagagttctattactaaacacagcatgactcaaaaaacttagcaattctgaaggaaagtccttggggtcttctacctttctcttcttttttggaggagtagaatgttgagagtcagcagtagcctcatcatcactagatggcatttcttctgagcaaaacaggttttcctcattaaaggcattccaccactgctcccattcatcagttccataggttggaatctaaaatacacaaacaattagaatcagtagtttaacacattatacacttaaaaattttatatttaccttagagctttaaatctctgtaggtagtttgtccaattatgtcacaccacagaagtaaggttccttcacaaagatccggaccaaagcggccatcgtgcctccccactcctgcagttcgggggcatggatgcgcggatagccgctgctggtttcctggatgccgacggatttgcactgccggtagaactccgcgaggtcgtccagcctcaggcagcagctgaaccaactcgcgaggggatcgagcccggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgaagctttttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagccggatctgcaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccaatgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgaggaactttcggcggctttgctgtgcgacaggctcacgtctaaaaggaaataaatcatgggtcataaaaattatcacgttgtcggcgcggcgacggatgttctgtatgcgctgttttccgttggccgttgctgtctggtgatctgccttctaaatctgcacagccgaattgcgcgagcttggttttgctgaaaccgacacacagcaactgaataccagaaagaaaatcactttgcctttctgacatcagaagggcagaaatttgccgttgaacacctggtcaatacgcgttttggtgagcagcaatattgcgcttcgatgagccttggcgttgagattgatacctctgctgcacaaaaggcaatcgaccgagctggaccagcgcattcgtgacaccgtctccttcgaacttattcgcaatggagtgtcattcatcaaggacngcctgatcgcaaatggtgctatccacgcagcggcaatcgaaaaccctcagccggtgaccaatatctacaacatcagccttggtatcctgcgtgatgagccagcgcagaacaaggtaaccgtcagtgccgataagttcaaagttaaacctggtgttgataccaacattgaaacgttgatcgaaaacgcgctgaaaaacgctgctgaatgtgcggcgctggatgtcacaaagcaaatggcagcagacaagaaagcgatggatgaactggcttcctatgtccgcacggccatcatgatggaatgtttccccggtggtgttatctggcagcagtgccgtcgatagtatgcaattgataattattatcatttgcgggtcctttccggcgatccgccttgttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgagctttttggcctctgtcgtttcctttctctgtttttgtccgtggaatgaacaatggaagtcaacaaaaagcagacgtatctagacacgtctgaagctagcttcgaggaactttcggcggctttgctgtgcgacaggctcacgtctaaaaggaaataaatcatgggtcataaaaattatcacgttgtcggcgcggcgacggatgttctgtatgcgctgttttccgttggccgttgctgtctggtgatctgccttctaaatctgcacagccgaattgcgcgagcttggttttgctgaaaccgacacacagcaactgaataccagaaagaaaatcactttgcctttctgacatcagaagggcagaaatttgccgttgaacacctggtcaatacgcgttttggtgagcagcaatattgcgcttcgatgagccttggcgttgagattgatacctctgctgcacaaaaggcaatcgaccgagctggaccagcgcattcgtgacaccgtctccttcgaacttattcgcaatggagtgtcattcatcaaggacngcctgatcgcaaatggtgctatccacgcagcggcaatcgaaaaccctcagccggtgaccaatatctacaacatcagccttggtatcctgcgtgatgagccagcgcagaacaaggtaaccgtcagtgccgataagttcaaagttaaacctggtgttgataccaacattgaaacgttgatcgaaaacgcgctgaaaaacgctgctgaatgtgcggcgctggatgtcacaaagcaaatggcagcagacaagaaagcgatggatgaactggcttcctatgtccgcacggccatcatgatggaatgtttccccggtggtgttatctggcagcagtgccgtcgatagtatgcaattgataattattatcatttgcgggtcctttccggcgatccgccttgttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgagctttttggcctctgtcgtttcctttctctgtttttgtccgtggaatgaacaatggaagtcaacaaaaagcagagcttatcgatgataagcggtcaaacatgagaattc

SEQ ID NO: 6 - DNA, 31567 bp, artificial sequence
  synthetic polynucleotide
gtcgagcggccgctcgactctagagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaagctttaggctgaaagagagatttagaatgacagaatcatagaacggcctgggttgcaaaggagcacagtgctcatccagatccaaccccctgctatgtgcagggtcatcaaccagcagcccaggctgcccagagccacatccagcctggccttgaatgcctgcagggatggggcatccacagcctccttgggcaacctgttcagtgcgtcaccaccctctgggggaaaaactgcctcctcatatccaacccaaacctcccctgtctcagtgtaaagccattcccccttgtcctatcaagggggagtttgctgtgacattgttggtctggggtgacacatgtttgccaattcagtgcatcacggagaggcagatcttggggataaggaagtgcaggacagcatggacgtgggacatgcaggtgttgagggctctgggacactctccaagtcacagcgttcagaacagccttaaggataagaagataggatagaaggacaaagagcaagttaaaacccagcatggagaggagcacaaaaaggccacagacactgctggtccctgtgtctgagcctgcatgtttgatggtgtctggatgcaagcagaaggggtggaagagcttgcctggagagatacagctgggtcagtaggactgggacaggcagcgttcatacaatcgtcaaatcatgaaggctggaaaagccctccaagatccccaagaccaaccccaacccacccaccgtgcccactggccatgtccctcagtgccacatccccacagttcttcatcacctccagggacggtgacccccccacctccgtgggcagctgtgccactgcagcaccgctctttggagaaggtaaatcttgctaaatccagcccgaccctcccctggcacaacgtaaggccattatctctcatccaactccaggacggagtcagtgaggatggggctctagagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaagctttaggctgaaagagagatttagaatgacagaatcatagaacggcctgggttgcaaaggagcacagtgctcatccagatccaaccccctgctatgtgcagggtcatcaaccagcagcccaggctgcccagagccacatccagcctggccttgaatgcctgcagggatggggcatccacagcctccttgggcaacctgttcagtgcgtcaccaccctctgggggaaaaactgcctcctcatatccaacccaaacctcccctgtctcagtgtaaagccattcccccttgtcctatcaagggggagtttgctgtgacattgttggtctggggtgacacatgtttgccaattcagtgcatcacggagaggcagatcttggggataaggaagtgcaggacagcatggacgtgggacatgcaggtgttgagggctctgggacactctccaagtcacagcgttcagaacagccttaaggataagaagataggatagaaggacaaagagcaagttaaaacccagcatggagaggagcacaaaaaggccacagacactgctggtccctgtgtctgagcctgcatgtttgatggtgtctggatgcaagcagaaggggtggaagagcttgcctggagagatacagctgggtcagtaggactgggacaggcagctggagaattgccatgtagatgttcatacaatcgtcaaatcatgaaggctggaaaagccctccaagatccccaagaccaaccccaacccacccaccgtgcccactggccatgtccctcagtgccacatccccacagttcttcatcacctccagggacggtgacccccccacctccgtgggcagctgtgccactgcagcaccgctctttggagaaggtaaatcttgctaaatccagcccgaccctcccctggcacaacgtaaggccattatctctcatccaactccaggacggagtcagtgaggatggggctctagaggatccctcgacctgcaggtcaacggatcacaacaaactggaaaattcttcaagagaagaataccagaccaccctacctgcttcctgagaaatctgtttgctgctcagaagcaacagttagaaccagacatggaacaacagactggttccaaatcaggaaaggagtatgtcaaggctgtatatcgtcaccctgcttatttaacttatatgcatagtacataatacaaaatgccaggctggatgaatcgcaagctggaatcaagatttctgggagaaatatcaataaacgagatacaaagatgacaccacacttatggcagaaaactaagaagaactaaagagcctcttgatgaaagtgaaagaggagagtgaaaaagccagcttaaaacccaacattcaaaatcaagatcatcatttcatggcaaataaatggggaaacaatggaaacagtgagagactttattttcttgggctccaaaatcactgcagattgtgactacagccatgattaaaagatgcttgctccttggaagagaagctattaccaaactagaaagcatattaaaaagcagagacgttactttgctgactaagttctgtctagtcaaacctatggtttttccagtagtcatatatggatgtgagttgaactataaagaaagctgagcaccaaagaattgatgcttttgaaatttggtgttggagaagtctcttgagagtcccttgacctgcaaggagatccaaccagtccatcctaaaggaaatcagtcctgaatattcattggaaggactgatgctgaaattgaagtttcaatactttgaccacctaatgcgaagagccaactcactagaaaagaccccgatgttgggaaaaattgaagccaggaagagaagtgaatgacagaggatgagatggttggatggcatcgttgactgaatggacatgagtctgatcaagttccgggagacagcaaaggacagggctgcctggtctgctgcagtccatggggttgcaaagagtcggactcaactgagtaactaaacaacaaccaagcagtagaaaaataaataaaatttgtctctgagatctcagtacctctttctgtgcatatccgtctcctgttattgtactttgtcttctgcttgtaataaagctgtcctgttagtaaaatctgtttgggtcctctgaattcttttagctatcaaaaatggaaggtgattattgtgcaatgtccacctctgagtaatatacagagaataaaagaagggagaaattatgtgcaagttctctctcatctcctgcttctcatttaaaagattctacctcagtgggggctaaaactccacatttaacagtagcaaaaaccaatattccatagcttcttaggaaaccattttttatactcttgtatgtaattacattcaagctcaaaagcaaagaagtgattctgcgttggtgaaggcccaaccatagaaaagaggaagaaaataggccacatactgtgcttcccccatagctcagttggtaaagaatctacctacaatgcaggaggcctgggcttgatccctgggtaagggagatcccctggagaaggaaatggtaacccactccagtactcttgcctgtaaaatcccatggacggaggagcctggcaggctacagccttggggtggcaagagttggacatggattaacaactaaaccactgccaccactccacatactgagtgctccccagtggcactagtggtaaagaacccacctgccggtgcagaagacattaaagacactggctctatccctgcttgggaagtagggaagatcccctagagagggaaatagcaacccactccagaattcttgcctggaaaatcccatgaatgaagactggcgggctgtagtaactggggtcacaagagttaaacatgatttagcaactaaacatcaccacattaaaaaaattaccacaaaatagtcatattccaggctaaggggaataatagcactagtacctgagagaactttctcagattctctgtcaagttcttccttctctcagataaccagtagtctagtttacctcatcagatattaactactcatcgattctaaattatctaattatgggggggggcactacattgcattatattttgtgtccattgactatcactcaatttatttataaaaaattcatccatgttgttttctgtgacagtaactcattcacattaattgtaatatctcattgcattgtatactacaatttatttatacaaaatactattattcacacttctgttgattttaatttggaacatcaacaataacgtggctgagaagcttctttctttagtatattgttaaggatttccttgatcaagattttacctacttttctggtccaattggtgagagacagtcataaggaaatgctgtgtttattgcacaatatgtaaagcatcttcctgagaaaataaaagggaaatgttgaatgggaaggatatgctttcttttgtattccttttctgagaaatcagactttttcacctgtggccttggccaccaaaagctaacaaataaaggcatatgaagtagccaaggccttttctagttatatctatgacactgagttcatttcatcatttattttcctgacttcctcctgggtccatatgagcagtcttagaatgaatattagctgaataatccaaatacatagtagatgttgatttgggttttctaagcaatccaagacttgtatgacagtaagatgtattaccatccaacacacatctcagcatgatataaatgcaaggtatattgtgaagaaaaatttttaattatgtcaaagtgcttactttagaaggtcatctatctgtcccaaagctgtgaatatatatattgaaggtaatgaatagatgaagctaaccttgtaaaaatgagtagtgtgaaatacaactacaattatgaacatctgtcactaaagaggcaaagaaacttgaagattgcttttgcaaatgggctcctattaataaaaagtacttttgaggtctggctcagactctattgtagtacttagggtaagaccctcctcctgtatgggctttcattttctttcttgcttccctcatttgcccttccatgaatactagctgataaacattgactataaaagatatgaggccaaacttgagctgtcccattttaataaatctgtataaataatatttgttctacaaaagtattatctaaataaatgttactttctgtcttaaaatccctcaacaaatccccactatctagagaataagattgacattccctggaatcacagcatgctttgtctgccattatctgacccctttctctttctctcttctcacctccatctactcctttttccttgcaattcatgacccagattcactgtttgatttggcttgcatgtgtgtgtgctgagttgcgtctgactgttatcaaccccatgaatgatagtccaccaggctctactgtccatgaaattttccagtcaagaatactggagtggattgcatttcctactccatttgattaatttagtgacttttaaatttctttttccatattcgggagcctattcttcctttttagtctatactctcttcactcttcaggtctaaggtatcatcgtgtgcttgttagcttgttactttctccattatagcttaagcactaacaactgttcaggttggcatgaaattgtgttctttgtgtggcctgtatatttctgttgtgtattagaatttaccccaagatctcaaagacccactgaatactaaagagacctcattgtggttacaataatttggggactgggccaaaacttccgtgcatcccagccaagatctgtagctactggacaatttcatttcctttatcagattgtgagttattcctgttaaaatgctccccagaatttctggggacagaaaaataggaagaattcaaatccactgttggttttatttcaaaccacaaaattagcatgccattaaatactatatataaacagccactaaatcagatcattatccattcagcttctccttcacttcttctcctctactttggaaaaaaggtaagaatctcagatataatttcagtgtatctgctactcatctttattttggactaggttaaaatgtagaaagaacataattgcttaaaatagatcttaaaaataagggtgtttaagataaggtttacactattttcagcagatatgttaaaaaatagaagtgactataaagacttgataaaaattatagtgactgcaaatgttttaggaatataataagatataataacagtggttgctattttctttagcacaagactagttaacaggctgtattaaaagatcttttcttgaattaaatattttcaatttgattaaacctacctcagccataaaggcaagcacatttcatttatactatggggatttgaataattattactgaagaagctctaccaacaaaaagtttatagagctatcatatttagtcaagagataaagagggttgttaggatatatatgctatttgaaaggtatttataaaagaagagtatatttatcaaaatttctcagaacatccaaatttcaagtttatcatttatcttacaatatttcaaaaatattaaaatagatacatgaaatacagaagtaaattaaagagaaagtattttacttggtaaaaaaattctaggttggacagagagtgccaggaaacaaaaacaatgaaaaatgtgacctgacaggaattatagctcaaagtatagtagtaagtaatgaaatggcttaaaaattggtatataaaatgctagttataaaataaacaaaatgcaataatatcctccctacatgtaatgaattctaggtattatgctcttttttgaagtcttgacaataaaaatttttttagaagtttataggcatcttgaataaagtgaaacaaattaagaattagtatccatgagaaaaatatagaacaattttcctaatttagtttgaaaatctgggattgaagatgtgtgtcaagagatgttggtggcaagaacatttttttttcaagaacttataaaaatgcaacaaaacaaaccatttaatacattttggtcaaaatcaataatgtattttattttatgctccaaggagcataaaattggggactgggcaagagaaactgacaccctggtaaattaccaagagataagtacacagttctatgtagagaaaataagcatagtgtatgatctctaaaattatgtgagacaaaggagagatgacattaggcatgtggggatgaagactgagtagagaagaaacaatctaatcagtccaagaaaacatctcgatcagtggaacaaatagaagaaatgctaaaatgaaacagaagtcttactggaaataaaagatatgcataagacaaaaattcatgaaaatcacttagtttagcagagaaaagataaaaataaagtatgaccttcttcatatacattgtttgatcatatgcacctcaataaaactgagtctccaacagaaatgaaacattaatattttgttcactgctctaatcccagaatctaagcgatatctggcaataaaaataataaatatatattttttaataaatgaatcaaccacttaatttttctgtaaatatctgtaacttctcttctgtctttccaaaaacactcataagtactgtgaatgagatgaaaaagagtgaagtaggatataggctgttagcagaaaacatctgaatggctggcagtgaaacattaacttgaaatgtaagattaatgagtaatagtaaattttaaccttggccatatgataaaatgttcattaatatttttctagaatacagggctttttgtttttgccatgaggtttgcaggatcttggttccctgaccagggatcaaacctgcactcccctggaagcatggagtcttggacatttgtattatacactatctttggttccttttaaagggaagtaattttacttaaataagaaaatagattgacaagtaatacgctgtttcctcatcttcccattcacaggaatcgcggatcctcgagaccgccatggccgtgctgggcctgctgttctgcctggtgaccttccccagctgcgtgctgtcccaggtgcagctgaagcagagcggccctggcctggtgcagccctctcagtccctctccattacctgtaccgtgtccggcttctctctcacaaattatggagtccattgggtgcgccagagccccggcaagggcctggagtggctgggcgtgatctggtccggcggcaacaccgactacaacacccccttcaccagcagactgagcatcaacaaggacaacagcaagagccaggtgttcttcaagatgaacagcctgcagagcaacgacaccgccatctactactgcgctcgcgctctcacctactacgactacgagttcgcctactggggccagggcaccctggtgaccgtgtccgccgccagcaccaagggccccagcgtgttccccctggcccccagcagcaagagcaccagcggcggcaccgctgccctgggctgtctggtgaaggactacttccccgagcccgtgaccgtgtcttggaactctggcgccctgacctccggcgtgcacacatttccagctgtcctccagagttccggactgtattccctcagttctgtcgtgacagtcccttcctctagcctgggcacccagacctacatctgcaacgtgaaccacaagcccagcaacaccaaggtggacaagagagtggagcccaagagctgcgacaagacccacacctgcccaccatgtcctgctccagagctgctggggggaccctccgtgttcctgttcccccccaagcccaaggacaccctgatgatcagcaggacccccgaggtgacctgcgtggtggtggacgtgtcccacgaggaccctgaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctgccagcccccatcgaaaagaccatcagcaaggccaagggccagcctcgcgagccccaggtgtacaccctgcccccctcccgcgaggagatgaccaagaaccaggtgtccctgacctgcctggtgaagggcttctaccccagcgatatcgccgtggagtgggagagcaacggccagcccgagaacaactacaagaccaccccccctgtgctggacagcgacggcagcttcttcctgtacagcaagctgaccgtggacaagtccaggtggcagcagggaaatgtcttttcttgttctgtcatgcatgaagctctgcacaaccactacacccagaagtccctgagcctgtcccccggcaagtgatagctcgaggatccggacccttccctattcttgtaagtctaaatttactaactgtgctgtttaacttctgatgtttgtatgatatttgagtaattaagagccctacaaaaaaatcaataatgaatggttccaaaataagcatagctgagattaatgattctcagcattagttataaatagaataagctggaaaaccttcacctcccctccaccaccagatctcaatgtctaggcttacccatggagattctgattaactgttctttctatgtagaagaaacttattgggaagaaataatataatggactatgatttaattggtctgttgagaatttagatgaaggggattaagttacaataaagccagaatttaacttgataatctcatttggctaagaataacaaacctaagaaggtttgctattttctacaattttgaagttttccttatgcacaattatttcaccacatgactcatttcacatcttgtttttgatatatgagcatatgagggcaaaatactgaagatgcttatttcaatactcagggaaaattttcttgccaaaaggcaagaattgtataattcattcacttattttattttttttaatttttaaggtctaagaggatttcaaagtgaatgccccctcctcacttttggtaagctttaggagattggaggcagactgatcatttttatagttaatatcttttacatttcatcttcctggataagccccaatagtagcaatttctatcagtataccagcataaagattagttttaaatttattttcagtgattgactgttatttactgacctgaaattatgtatctgttatatttcaaataatgcaaaactgtatatatatggtgttgacagatttgattggttttctttcaattgcctatatccttattattgattgtaatcatttatagaaaaaacaaaataatttcttatacttttatgtaaacctgttagagcttattttaaagatcaactgcattcacatttctaatctagtcattatgagcttcaattgttttatctcacttaaaatttatatattgtcttttaattcatgagtcaaaatacaatctcacagtccagatatgggacttaaaaggggaatagcatatagttttgatattcttaaagatatacatctttttgtgatcatgattcagcagacattttaataaaacaattccaagtgagccgacacttggtcctagaggaatttttataaccttaagataaggcacagcatggtgtttttgtaataagatttcttttatgaaaaagtcacaccaaaattggaaatggggtgagatgaagagttataacatataactaaatggacatttgttctctattccacagaattgactgcgactggaaatatggcaacttttcaatccttgcatcatgctactaagataatttttaaatgagtatacatggaacaaaaaatgaaactttattcctttatttatattatgctttttcatcttaatttgaatttgagtcataaaccatatactttcaaaatgttaattcaacattagcataaaagttcaattttaacttggaaatatcatgaacatatcaaattatgtataaaaataatttctggaattgtgattattatttctttaagaatctatttcctaaccagtcatttcaataaattaacccttaggcatatttaagttttcttgtctttattatatttttaaaaatgaaattggtctctttattgttaacttaaatttatctttgatgttaaaaatagctgtggaaaattaaaattgaatagaattctttgaattgagttccaaaggatatcaaaaagtgagggaaaagatagggtgagcctatgctgcatatgtccttagaaagtcttggtttatacctgttacctaagttaaacaattatacttgttcctttcactctcgaaagtacccagcattggatgttaaattttatagtcatcctagacaaaaaaaaaaaaaaaaacaaacaaccctcaaatgtgatatctgaatcacagctctacagtgtggtagctaagtggtgctgtgtaagttagtctccaagagattccatttctacatttataaacagtcaatttaaggtgttttattgaagttttaatgtgaaaagtgcactatatggtgcatgataggagttcctggttgaatctcatttctgacatcactgacaccagtgcagcaaggactagtgttacaatcagaaggagctgagttgtgtaattttagccattaatgcccaagagactagaacttacacaaagctctaatatccattgtctctgtctgtggagtaattatttcattgccatgaattatctgtctgtcatatcctgcatttttatacatgattcagttcccttcagttcacacaatgacttgtctaatttcatctttcctgcatcctccatgttttcctcacttcaggattaagtgaagccgtacttaggcacaatatttcttatctttaaagaaaaattccatctttgagagttgttattgttcagtcactaggtcatgtccaactctttgtgaccccatgcactgcagcatgccaggcttccctgcccttcgctctctcctggagtttgctcagactcatgtagattgagtcggtgatggtatccaactatctcatcaactgttgtgcccttctcctcctaccctcagtctttaccagcatcagagtctttctcagattcttcaggttattatataacaactatcataaaaggagtatctaaatggctgtgtccattatttcacatgttattctctctttaacttgctccaatcccaattttatccctatgggaactgctttattgaagatcaccaacaacttttattttactaatcgttttgttttacccaacctctcagtgagtgttatgaggtagagttgactatttcttcattttgaaatattacgcttcatttcatttgatatcctaaagctcataaggtgtggtttttctcttaactcactagacacttctttgaagtctctcttctggcattttctccttttccaaaattttaatggttggagtaccctagattttagccttaatttgtttgatgttgttcagttccattctcagctcagagcttccaactgtatgtctccaaacttactcgttttgtaaactccaaactcatgcactcaactgcattcttgacctccacactgaattatctaattaatgtcctaaatctggcatgaccaagcatacatttttgtctgaatccagtccccaacttgctcaaaatttaattaaacgtaattcagttacaaaggcagctgatattgtatgcaatagacctgaatgggaacttcacaaaagaagttatcttaattgtcaataaaaacatgaaaaatactctacatcatcaatcttcagaaaaatgcaaattaaaggtgcctaataatatcatgacacaaccgtcagaatgactgaaatgaaaagaattgtaatacagttcagttcagttcagttactcagtcgtctccaactctttgtgaccccatgaactgcagcatgacagaccttcctgtccatcaccaactcccagagtttactcagactatgtccattgagttgatgatgccatccaaccatctcatcctctgtcgtccccttctcctcctgccctcagtctttcccagcatcagggtcttttccaatgagtcagctcttcgcatcaggtggctaaagtattggagtttcagcttcaacatcagtccttctaattaacacccaggactgatctcttttaggatggactagttggatctccttgcagtccaagggactctcaagagtcttctccaacaccacagttcaaaagcatcaattccttggcactcagctttccttatagtccatgtctcacatccacacatgactattggaaaaaccatagccttgactaggtggacctttgttgacaaagtaatgtctctgctttttaatatgttgtctagattggtcataactttccttccaagaagtaattgtcttttaatttcatggctgcagtcaccatctgcagtgattttggagccccaaaatataaagtcagctgctgtttccactgttgccccatctaccccatctatttgccatgaagtgatgggactggatgccactatcttagttttctgaatgttgagctttaagccagcctttttactctcctctttcactttcatcaagaggctctttagttcctcttcactttctgccataagggtggtgtcatctgcatatctgaggttattgatatttctcttggcaattttgattccagcctgcacttcttccagcccagtgtttctcatgatgtactctgcatataaattaaataagcagagtgacaatatacagccttgacatactctttttcctatttggaaccagtctgttgttccatgtccagttctaactgttgtttcctgacctgcatacaggtttctcaagaggcaagtcaggtggtctggtattctcacctgtttcagaattttccacagtttattgtgatccacacagtcaaaggctttggcatagccaataaagcagaaagagatgtttttctggaactctcttacttttttgatgatccagtggatgttggcaatttgatctctggttcctctgccttttctaaaaccagctttaacatctggaagttcatggttcacgtaatacaaaatgtaatacaaaatgtctgcaaaaacaaaggaatgaaaagtaatgctaaaaaatgttaatatttacagaaatttttatagtagtaaagaattcacctgcaatacaggagaaccgggttagatccctgggttggaagacctcctggagaaggaaatggctacccaatctagtattcttgtctggagaaggcaagaatggacagagaagcccagcgggctatggtccatcgggtcacaaagagtcagaagctaccttgcacacagcaagcacggtgcgcgcgcgtgcacacacacacacacacacacacacagacacacacacactctaaaacatttacccaagcttgtccaatggaaaatcaaaaagccagcaatttaagatgacatcaggtaccactgtccaggtaagcctcagaacacaatgaccagtaagaagcaaagtgccatatgagcaactcgaatttttgcaatgttacctaagagcttccatttttataatgcaaaagaatttcatatggggaaattgtattagataaccctgaatgaggagcaagatatagtcaaagtaagatgctctagtactattttttataagcatgatttgttcagccaaaggttttttcccatatggccaatgaactgaaatatgcagtcctgagatttgcatatatttctagctgaaaccaagtaaataatatcctcaagaaagaaatcaatagaaaagttggatgaagagtacaataaagggaccaaaaatattcagaaataagaactagaggagatattgggaaatccctggtgagtccagtttaggattttgtactttcactgcagttggcatggatataatccctcactggggaactaagatcccataagctgtgttggattgccaaaaaaataaatattaagagatatcattcatagaatattttaaagatattttagagaagaggaaattaaggatgtgagaatttgtattactttttcaagatactaaagctatttagagatagagctgttactaaaaacttcagtttcctaaaaattatttgaagcactgtttaataaattccaaaatatagaggaaggaaaacaaaatactgaggattcatataatgattcagatttagaaacaatataacacagaattagtgaattctgacaaattattaggtaggagtagatagttcagcattactcgtatagatggagtatttaatcctttccatgagattatccaaatataataatttcgtatctatgtgaagtataactattaagattactttataaagtaaatcaagaaccagagaataagaaaaatgttttgtgaaccagcagatactatgaacacataaaactcagaaccctgattcctaagacacacagctaatcctgattattcttcctttacatgtgaccatagaacttcacacaagttcaagatacatttgttgagcacatcagtatcagttcagtcactcagtcatgtccgaatctttgtgaccttgtggactgcagcacgccaggctttcctgtccaccaccaacccctggagcttactcaaactcatgtccattgagtcagtgatcccatccaaccatctcatcctctgtcatcctcttctcctgccttcaatctttcccagacattggagtcttttccaatgagtcagatcttcacattaggtggccaaagtataggagtttcagcttcagcatcaatccttccaatgaatattccttgatgtacccctttcgcagtttggaaccagtctgttgttccatgtccagttctaactgctgcttctggacctgtatacagatttctcaggaggcaggtaaagtggtctggtattcccatctcttgaagaattttccacagtttattgtgatccacacaatcaaaggctttagcgtagtcaataaagcagatgtttttctggaactctcgtgcttttttgatgatccaatggatgttggcaatttgatctctggttcctctgccttttctaaatccagcttgaacatctggaagttcatggtccacgtactgttgaagcctggcttggagaattttgagagttattttgctagcatgtgagatgagtgcaatcatgtgggtgtttgaacatactttgtcattgcttttctttgggattgtggcagtcctgtggccactgctgagttttccaaatttgctgacatattgagtgcagcactttcacagcatcaccttttaagatttgaaatagctcaactggaattccatcacctccactagctttgttcatagtgaggctttctaaggccgtttgactttgcattccagggtgtctggctctaggtgagtgatccgttgacctgcagcggccctcgactctagagggacagcccccccccaaagcccccagggatgtaattacgtccctcccccgctaggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaagctttaggctgaaagagagatttagaatgacagaatcatagaacggcctgggttgcaaaggagcacagtgctcatccagatccaaccccctgctatgtgcagggtcatcaaccagcagcccaggctgcccagagccacatccagcctggccttgaatgcctgcagggatggggcatccacagcctccttgggcaacctgttcagtgcgtcaccaccctctgggggaaaaactgcctcctcatatccaacccaaacctcccctgtctcagtgtaaagccattcccccttgtcctatcaagggggagtttgctgtgacattgttggtctggggtgacacatgtttgccaattcagtgcatcacggagaggcagatcttggggataaggaagtgcaggacagcatggacgtgggacatgcaggtgttgagggctctgggacactctccaagtcacagcgttcagaacagccttaaggataagaagataggatagaaggacaaagagcaagttaaaacccagcatggagaggagcacaaaaaggccacagacactgctggtccctgtgtctgagcctgcatgtttgatggtgtctggatgcaagcagaaggggtggaagagcttgcctggagagatacagctgggtcagtaggactgggacaggcagctggagaattgccatgtagatgttcatacaatcgtcaaatcatgaaggctggaaagcctccaagatccccaagaccaaccccaacccacccaccgtgcccactggccatgtccctcagtgccacatccccacagttcttcatcacctccagggacggtgacccccccacctccgtgggcagctgtgccactgcagcaccgctctttggagaaggtaaatcttgctaaatccagcccgaccctcccctggcacaacgtaaggccattatctctcatccaactccaggacggagtcagtgaggatggggctctagagggacagcccccccccaaagcccccagggatgtaattacgtccctcccccgctaggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaagctttaggctgaaagagagatttagaatgacagaatcatagaacggcctgggttgcaaaggagcacagtgctcatccagatccaaccccctgctatgtgcagggtcatcaaccagcagcccaggctgcccagagccacatccagcctggccttgaatgcctgcagggatggggcatccacagcctccttgggcaacctgttcagtgcgtcaccaccctctgggggaaaaactgcctcctcatatccaacccaaacctcccctgtctcagtgtaaagccattcccccttgtcctatcaagggggagtttgctgtgacattgttggtctggggtgacacatgtttgccaattcagtgcatcacggagaggcagatcttggggataaggaagtgcaggacagcatggacgtgggacatgcaggtgttgagggctctgggacactctccaagtcacagcgttcagaacagccttaaggataagaagataggatagaaggacaaagagcaagttaaaacccagcatggagaggagcacaaaaaggccacagacactgctggtccctgtgtctgagcctgcatgtttgatggtgtctggatgcaagcagaaggggtggaagagcttgcctggagagatacagctgggtcagtaggactgggacaggcagctggagaattgccatgtagatgttcatacaatcgtcaaatcatgaaggctggaaagcctccaagatccccaagaccaaccccaacccacccaccgtgcccactggccatgtccctcagtgccacatccccacagttcttcatcacctccagggacggtgacccccccacctccgtgggcagctgtgccactgcagcaccgctctttggagaaggtaaatcttgctaaatccagcccgaccctcccctggcacaacgtaaggccattatctctcatccaactccaggaacggagtcagtgaggatggggctctagaggatccctcgacatctatgtcgggtgcggagaaagaggtaatgaaatggcactcgacggtatcgataagcttgatatcgaattctaccgggtaggggaggcgcttttcccaaggcagtctggagcatgcgctttagcagccccgctggcacttggcgctacacaagtggcctctggcctcgcacacattccacatccaccggtagcgccaaccggctcccttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagcttgnnnccttcgctttctgggctcagaggctgggaaggggtgggtccgggggcgggctcaggggcgggctcaggggcggggcgggcgcgaaggtcctccggagcccggcattctgcacgcttcaaaagcgcacgtctgccgcgctgttctcctcttcctcatctccgggcctttcgacctgcagccaatatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatcaattctctagagctcgctgatcagcctcgactgtgccttctagttgccagccatctgttgtttgcccctcccccgtgccttccnngaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgatcgatcgagttcgatcgactctagagggacagcccccccccaaagcccccagggatgtaattacgtccctcccccgctaggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaagctttaggctgaaagagagatttagaatgacagaatcatagaacggcctgggttgcaaaggagcacagtgctcatccagatccaaccccctgctatgtgcagggtcatcaaccagcagcccaggctgcccagagccacatccagcctggccttgaatgcctgcagggatggggcatccacagcctccttgggcaacctgttcagtgcgtcaccaccctctgggggaaaaactgcctcctcatatccaacccaaacctcccctgtctcagtgtaaagccattcccccttgtcctatcaagggggagtttgctgtgacattgttggtctggggtgacacatgtttgccaattcagtgcatcacggagaggcagatcttggggataaggaagtgcaggacagcatggacgtgggacatgcaggtgttgagggctctgggacactctccaagtcacagcgttcagaacagccttaaggataagaagataggatagaaggacaaagagcaagttaaaacccagcatggagaggagcacaaaaaggccacagacactgctggtccctgtgtctgagcctgcatgtttgatggtgtctggatgcaagcagaaggggtggaagagcttgcctggagagatacagctgggtcagtaggactgggacaggcagctggagaattgccatgtagatgttcatacaatcgtcaaatcatgaaggctggaaagcctccaagatccccaagaccaaccccaacccacccaccgtgcccactggccatgtccctcagtgccacatccccacagttcttcatcacctccagggacggtgacccccccacctccgtgggcagctgtgccactgcagcaccgctctttggagaaggtaaatcttgctaaatccagcccgaccctcccctggcacaacgtaaggccattatctctcatccaactccaggacggagtcagtgaggatggggctctagagggacagcccccccccaaagcccccagggatgtaattacgtccctcccccgctaggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaagctttaggctgaaagagagatttagaatgacagaatcatagaacggcctgggttgcaaaggagcacagtgctcatccagatccaaccccctgctatgtgcagggtcatcaaccagcagcccaggctgcccagagccacatccagcctggccttgaatgcctgcagggatggggcatccacagcctccttgggcaacctgttcagtgcgtcaccaccctctgggggaaaaactgcctcctcatatccaacccaaacctcccctgtctcagtgtaaagccattcccccttgtcctatcaagggggagtttgctgtgacattgttggtctggggtgacacatgtttgccaattcagtgcatcacggagaggcagatcttggggataaggaagtgcaggacagcatggacgtgggacatgcaggtgttgagggctctgggacactctccaagtcacagcgttcagaacagccttaaggataagaagataggatagaaggacaaagagcaagttaaaacccagcatggagaggagcacaaaaaggccacagacactgctggtccctgtgtctgagcctgcatgtttgatggtgtctggatgcaagcagaaggggtggaagagcttgcctggagagatacagctgggtcagtaggactgggacaggcagctggagaattgccatgtagatgttcatacaatcgtcaaatcatgaaggctggaaagcctccaagatccccaagaccaaccccaacccacccaccgtgcccactggccatgtccctcagtgccacatccccacagttcttcatcacctccagggacggtgacccccccacctccgtgggcagctgtgccactgcagcaccgctctttggagaaggtaaatcttgctaaatccagcccgaccctcccctggcacaacgtaaggccattatctctcatccaactccaggaacggagtcagtgaggatggggctctagaggatccgcggccgcgaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtcagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatctctagtcaaggcactatacatcaaatattccttattaacccctttacaaattaaaaagctaaaggtacacaatttttgagcatagttattaatagcagacactctatgcctgtgtggagtaagaaaaaacagtatgttatgattataactgttatgcctacttataaaggttacagaatatttttccataattttcttgtatagcagtgcagctttttcctttgtggtgtaaatagcaaagcaagcaagagttctattactaaacacagcatgactcaaaaaacttagcaattctgaaggaaagtccttggggtcttctacctttctcttcttttttggaggagtagaatgttgagagtcagcagtagcctcatcatcactagatggcatttcttctgagcaaaacaggttttcctcattaaaggcattccaccactgctcccattcatcagttccataggttggaatctaaaatacacaaacaattagaatcagtagtttaacacattatacacttaaaaattttatatttaccttagagctttaaatctctgtaggtagtttgtccaattatgtcacaccacagaagtaaggttccttcacaaagatccggaccaaagcggccatcgtgcctccccactcctgcagttcgggggcatggatgcgcggatagccgctgctggtttcctggatgccgacggatttgcactgccggtagaactccgcgaggtcgtccagcctcaggcagcagctgaaccaactcgcgaggggatcgagcccggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgaagctttttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagccggatctgcaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccaatgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgaggaactttcggcggctttgctgtgcgacaggctcacgtctaaaaggaaataaatcatgggtcataaaaattatcacgttgtcggcgcggcgacggatgttctgtatgcgctgttttccgttggccgttgctgtctggtgatctgccttctaaatctgcacagccgaattgcgcgagcttggttttgctgaaaccgacacacagcaactgaataccagaaagaaaatcactttgcctttctgacatcagaagggcagaaatttgccgttgaacacctggtcaatacgcgttttggtgagcagcaatattgcgcttcgatgagccttggcgttgagattgatacctctgctgcacaaaaggcaatcgaccgagctggaccagcgcattcgtgacaccgtctccttcgaacttattcgcaatggagtgtcattcatcaaggacngcctgatcgcaaatggtgctatccacgcagcggcaatcgaaaaccctcagccggtgaccaatatctacaacatcagccttggtatcctgcgtgatgagccagcgcagaacaaggtaaccgtcagtgccgataagttcaaagttaaacctggtgttgataccaacattgaaacgttgatcgaaaacgcgctgaaaaacgctgctgaatgtgcggcgctggatgtcacaaagcaaatggcagcagacaagaaagcgatggatgaactggcttcctatgtccgcacggccatcatgatggaatgtttccccggtggtgttatctggcagcagtgccgtcgatagtatgcaattgataattattatcatttgcgggtcctttccggcgatccgccttgttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgagctttttggcctctgtcgtttcctttctctgtttttgtccgtggaatgaacaatggaagtcaacaaaaagcagacgtatctagacacgtctgaagctagcttcgaggaactttcggcggctttgctgtgcgacaggctcacgtctaaaaggaaataaatcatgggtcataaaaattatcacgttgtcggcgcggcgacggatgttctgtatgcgctgttttccgttggccgttgctgtctggtgatctgccttctaaatctgcacagccgaattgcgcgagcttggttttgctgaaaccgacacacagcaactgaataccagaaagaaaatcactttgcctttctgacatcagaagggcagaaatttgccgttgaacacctggtcaatacgcgttttggtgagcagcaatattgcgcttcgatgagccttggcgttgagattgatacctctgctgcacaaaaggcaatcgaccgagctggaccagcgcattcgtgacaccgtctccttcgaacttattcgcaatggagtgtcattcatcaaggacngcctgatcgcaaatggtgctatccacgcagcggcaatcgaaaaccctcagccggtgaccaatatctacaacatcagccttggtatcctgcgtgatgagccagcgcagaacaaggtaaccgtcagtgccgataagttcaaagttaaacctggtgttgataccaacattgaaacgttgatcgaaaacgcgctgaaaaacgctgctgaatgtgcggcgctggatgtcacaaagcaaatggcagcagacaagaaagcgatggatgaactggcttcctatgtccgcacggccatcatgatggaatgtttccccggtggtgttatctggcagcagtgccgtcgatagtatgcaattgataattattatcatttgcgggtcctttccggcgatccgccttgttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgagctttttggcctctgtcgtttcctttctctgtttttgtccgtggaatgaacaatggaagtcaacaaaaagcagagcttatcgtgataagcggtcaaacatgagaattc

SEQ ID NO: 7 - DNA, 26621 bp, artificial sequence
  synthetic polynucleotide
artctcgactctagagggacagcccccccccaaagcccccagggatgtaattacgtccctcccccgctaggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaagctttaggctgaaagagagatttagaatgacagaatcatagaacggcctgggttgcaaaggagcacagtgctcatccagatccaaccccctgctatgtgcagggtcatcaaccagcagcccaggctgcccagagccacatccagcctggccttgaatgcctgcagggatggggcatccacagcctccttgggcaacctgttcagtgcgtcaccaccctctgggggaaaaactgcctcctcatatccaacccaaacctcccctgtctcagtgtaaagccattcccccttgtcctatcaagggggagtttgctgtgacattgttggtctggggtgacacatgtttgccaattcagtgcatcacggagaggcagatcttggggataaggaagtgcaggacagcatggacgtgggacatgcaggtgttgagggctctgggacactctccaagtcacagcgttcagaacagccttaaggataagaagataggatagaaggacaaagagcaagttaaaacccagcatggagaggagcacaaaaaggccacagacactgctggtccctgtgtctgagcctgcatgtttgatggtgtctggatgcaagcagaaggggtggaagagcttgcctggagagatacagctgggtcagtaggactgggacaggcagctggagaattgccatgtagatgttcatacaatcgtcaaatcatgaaggctggaaagcctccaagatccccaagaccaaccccaacccacccaccgtgcccactggccatgtccctcagtgccacatccccacagttcttcatcacctccagggacggtgacccccccacctccgtgggcagctgtgccactgcagcaccgctctttggagaaggtaaatcttgctaaatccagcccgaccctcccctggcacaacgtaaggccattatctctcatccaactccaggacggagtcagtgaggatggggctctagagggacagcccccccccaaagcccccagggatgtaattacgtccctcccccgctaggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaagctttaggctgaaagagagatttagaatgacagaatcatagaacggcctgggttgcaaaggagcacagtgctcatccagatccaaccccctgctatgtgcagggtcatcaaccagcagcccaggctgcccagagccacatccagcctggccttgaatgcctgcagggatggggcatccacagcctccttgggcaacctgttcagtgcgtcaccaccctctgggggaaaaactgcctcctcatatccaacccaaacctcccctgtctcagtgtaaagccattcccccttgtcctatcaagggggagtttgctgtgacattgttggtctggggtgacacatgtttgccaattcagtgcatcacggagaggcagatcttggggataaggaagtgcaggacagcatggacgtgggacatgcaggtgttgagggctctgggacactctccaagtcacagcgttcagaacagccttaaggataagaagataggatagaaggacaaagagcaagttaaaacccagcatggagaggagcacaaaaaggccacagacactgctggtccctgtgtctgagcctgcatgtttgatggtgtctggatgcaagcagaaggggtggaagagcttgcctggagagatacagctgggtcagtaggactgggacaggcagctggagaattgccatgtagatgttcatacaatcgtcaaatcatgaaggctggaaagcctccaagatccccaagaccaaccccaacccacccaccgtgcccactggccatgtccctcagtgccacatccccacagttcttcatcacctccagggacggtgacccccccacctccgtgggcagctgtgccactgcagcaccgctctttggagaaggtaaatcttgctaaatccagcccgaccctcccctggcacaacgtaaggccattatctctcatccaactccaggaacggagtcagtgaggatggggctctagaggatccctcgacctgcaggtcaacggatcacaacaaactggaaaattcttcaagagaagaataccagaccaccctacctgcttcctgagaaatctgtttgctgctcagaagcaacagttagaaccagacatggaacaacagactggttccaaatcaggaaaggagtatgtcaaggctgtatatcgtcaccctgattatttaacttatatgcatagtacataatacaaaatgccaggctggatgaatcgcaagctggaatcaagatttctgggagaaatatcaataaacgagatacaaagatacaccacacttatggcagaaaactaagaagaactaaagagcctcttgatgaaagtgaaagaggagagtgaaaaagccagcttaaaacccaacattcaaaatcaagatcatcatttcatggcaaataaatggggaaacaatggaaacagtgagagactttattttcttgggctccaaaatcactgcagattgtgactacagccatgattaaaagatgcttgctccttggaagagaagctattaccaaactagaaagcatattaaaaagcagagacgttactttgctgactaagttctgtctagtcaaacctatggtttttccagtagtcatatatggatgtgagttgaactataaagaaagctgagcaccaaagaattgatgcttttgaaatttggtgttggagaagtctcttgagagtcccttgaacctgcaaggagatccaaccagtccatcctaaaggaaatcagtcctgaatattcattggaaggactgatgctgaaattgaagattaacgttttggactcacctaatgcagaagagccaactcactagaaaagaccccatgttggcaaaaattgaagccaggaagagaagtgaatgacagaggatgagatggttggatggcatcgttgactgaatggacatgagtctgatcaagttccgggagacagcaaaggacagggctgcctggtctgctgcagtccatggggttgcaaagagtcggtctcaaatgagtaactaaacaacaaccaagcagtagaaaaataaataaaatttgtctctgagatctcagtacctctttctgtgcatatccgtctcctgttattgtactttgtcttctgcttgtaataaagctgtcctgttagtaaaatctgtttgggtcctctgaattcttttagctatcaaaaatggaaggtgattattgtgcaatgtccacctctgagtaatatacagagaataaaagaagggagaaattatgtgcaagttctctctcatctcctgcttctcatttaaaagattctacctcagtgggggctaaaactccacatttaacagtagcaaaaaccaatattccatagcttcttaggaaaccattttttatactcttgtatgtaattacattcaagctcaaaagcaaagaagtgattctgcgttggtgaaggcccaaccatagaaaagaggaagaaaataggccacatactgtgcttcccccatagctcagttggtaaagaatctacctacaatgcaggaggcctgggcttgatccctgggtaagggagatcccctggagaaggaaatggtaacccactccagtactcttgcctgtaaatcccatggacggaggagcctggcagctacagccttggggtggcaagagttggacatgattaacaactaaaccactgccaccactccacatactgagtgctccccagtggcactagtggtaaagaaccacctgccggtgcagaagacattaaagacactggctctatccctgcttgggaagtagggaagatcccctagagagggaaatagcaacccactccagaattcttgcctggaaaatcccatgaatgaagactggcgggctgtagtaactggggtcacaaagagttaaacatgatttagcaactaaacatcaccacattaaaaaaattaccaccaaaatagtcatattccaggctaaggggaataatagcactagtacctgagagaactttctcagattctctgtcaagttcttccttctctcatataaccagtagtctagtttacctcatcagatattaactactcatcgattctaaattatctaattatgggggggggcactacattgcattatattttgtgtccattgactatcactcaatttatttataaaaaattcatccatgttgtttctgtgacagtaactcattcacattaattgtaatatctcattgcattgtatactacaatttatttatacaaaatactattattcacacttctgttgattttaatttggaacatcaacaataacgtggctgagaagcttctttctttagtatattgttaaggatttccttgatcaagattttacctacttttctggtccaattggtgagagacagtcataaggaaatgctgtgtttattgcacaatatgtaaagcatcttcctgagaaaataaaagggaaatgttgaatgggaaggatatgctttcttttgtattccttttctgagaaatcagactttttcaccttggccttggccaccaaaagctaacaaataaaggcatatgaagtagccaaggccttttctagttatatctatgacactgagttcatttcatcatttattttcctgacttcctcctgggtccatatgagcagtcttagaatgaatattagctgaataatccaaatacatagtagatgttgatttgggttttctaagcaatccaagacttgtatgacagtaagatgtattaccatccaacacacatctcagcatgatataaatgcaaggtatattgtgaagaaaaatttttaattatgtcaaagtgcttactttagaaggtcatctatctgtcccaaagctgtgaatatatatattgaaggtaatgaatagatgaagctaaccttgtaaaaatgagtagtgtgaaatacaactacaattatgaacatctgtcactaaagaggcaaagaaacttgaagattgcttttgcaaatgggctcctattaataaaaagtacttttgaggtctggctcagactctattgtagtacttagggtaagaccctcctcctgtatgggctttcattttctttcttgcttccctcatttgcccttccatgaatactagctgataaacattgactataaaagatatgaggccaaacttgagctgtcccattttaataaatctgtataaataatatttgttctacaaaagtattatctaaataaatgttactttctgtcttaaaatccctcaacaaatccccactatctagagaataagattgacattccctggaatcacagcatgctttgtctgccattatctgacccctttctctttctctcttctcacctccatctactcctttttccttgcaattcatgacccagattcactgtttgatttggcttgcatgtgtgtgtgctgagttgcgtctgactgttatcaaccccatgaatgatagtccaccaggctctactgtccatgaaattttccagtcaagaatactggagtggattgcatttcctactccatttgattaatttagtgacttttaaatttctttttccatattcgggagcctattcttcctttttagtctatactctcttcactcttcaggtctaaggtatcatcgtgtgcttgttagcttgttactttctccattatagcttaagcactaacaactgttcaggttggcatgaaattgtgttctttgtgtggcctgtatatttctgttgtgtattagaatttaccccaagatctcaaagacccactgaatactaaagagacctcattgtggttacaataatttggggactgggccaaaacttccgtgcatcccagccaagatctgtagctactggacaatttcatttcctttatcagattgtgagttattcctgttaaaatgctccccagaatttctggggacagaaaaataggaagaattcatttcctaatcatgcagatttctaggaattcaaatccactgttggttttatttcaaaccacaaaattagcatgccattaaatactatatataaacagccactaaatcagatcattatccattcagcttctccttcacttcttctcctctactttggaaaaaaggtaagaatctcagatataatttcagtgtatctgctactcatctttattttggactaggttaaaatgtagaaagaacataattgcttaaaatagatcttaaaaataagggtgtttaagataaggtttacactattttcagcagatatgttaaaaaatagaagtgactataaatacttgataaaaattatagtgactgcaaatgttttaggaatataataagatataataacagtggttgctattttctttagcacaagactagttaacaggctgtattaaaagatcttttcttgaattaaatattttcaatttgattaaacctacctcagccataaaggcaagcacatttcatttatactatggggatttgaataattattactgaagaagctctaccaacaaaaagtttatagagctatcatatttagtcaagagataaagagggttgttaggatatatatgctatttgaaaggtatttataaaagaagagtatatttatcaaaatttctcaagaacatccaaatttcaagtttatcatttatcttacaatatttcaaaaatattaaaatagatacatgaaatacagaagtaaattaaagagaaagtattttacttggtaaaaaaattctaggttggacagagagtgccaggaaacaaaaacaatgaaaaatgtgacctgacaggaattatagctcaaagtatagtagtaagtaatgaaatggcttaaaaattggtatataaaatgctagttataaaataaacaaaatgcaataatatcctccctacatgtaatgaattctaggtattatgattatgctcttttttgaagtcttgacaataaaaatttttttagaagtttataggcatcttgaataaagtgaaacaaattaagaattagtatccatgagaaaaatatagaacaattttcctaatttagtttgaaaatctgggattgaagatgtgtgtcaagagatgttggtggcaagaacatttttttttcaagaacttataaaaatgcaacaaaacaaaccatttaatacattttggtcaaaatcaataatgtattttattttatgctccaaggagcataaaattggggactgggcaagagaaactgacaccctggtaaattaccaagagataagtacacagttactatagtagaaaataagcatagtgtatgatctctaaaattatgtgagacaaaggagagatgacattaggcatgtggggatgaagactgagtagagaagaaacaatctaatcagtccaagaaaacatctcgatcagtggaacaaatagaagaaatgctaaaatgaaacagaagtcttactggaaataaaagatatgcataagacaaaaattcatgaaaatcacttagtttagcagagaaaagataaaaataaagtatgaccttcttcatatacattgtttgatcatatgcacctcaataaaactgagtctccaacagaaatgaaacattaatattttgttcactgctctaatcccagaatctaagcgatatctggcaataaaaataataaatatatattttttaataaatgaatcaaccacttaatttttctgtaaatatctgtaacttctcttctgtctttccaaaaacactcataagtactgtgaatgagatgaaaaagagtgaagtaggatataggctgttagcagaaaacatctgaatggctggcagtgaaacattaacttgaaatgtaagattaatgagtaatagtaaattttaaccttggccatatgataaaatgttcattaatatttttctagaatacagggctttttgtttttgccatgaggtttgcaggatcttggttccctgaccagggatcaaacctgcacaccagggatcaaacctgcactcccctggaagcatggagtcttggacatttgtattatacactatctttggttccttttaaagggaagtaattttacttaaataagaaaatagattgacaagtaatacgctgtttcctcatcttcccattcacaggaatcgcggatcctcgagaccgccatggccgtgctgggcctgctgttctgcctggtgaccttccccagctgcgtgctgtcccaggtgcagctgaagcagagcggccctggcctggtgcagccctctcagtccctctccattacctgtaccgtgtccggcttctctctcacaaattatggagtccattgggtgcgccagagccccggcaagggcctggagtggctgggcgtgatctggtccggcggcaacaccgactacaacacccccttcaccagcagactgagcatcaacaaggacaacagcaagagccaggtgttcttcaagatgaacagcctgcagagcaacgacaccgccatctactactgcgctcgcgctctcacctactacgactacgagttcgcctactggggccagggcaccctggtgaccgtgtccgccgccagcaccaagggccccagcgtgttccccctggcccccagcagcaagagcaccagcggcggcaccgctgccctgggctgtctggtgaaggactacttccccgagcccgtgaccgtgtcttggaactctggcgccctgacctccggcgtgcacacatttccagctgtcctccagagttccggactgtattccctcagttctgtcgtgacagtcccttcctctagcctgggcacccagacctacatctgcaacgtgaaccacaagcccagcaacaccaaggtggacaagagagtggagcccaagagctgcgacaagacccacacctgcccaccatgtcctgctccagagctgctggggggaccctccgtgttcctgttcccccccaagcccaaggacaccctgatgatcagcaggacccccgaggtgacctgcgtggtggtggacgtgtcccacgaggaccctgaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctgccagcccccatcgaaaagaccatcagcaaggccaagggccagcctcgcgagccccaggtgtacaccctgcccccctcccgcgaggagatgaccaagaaccaggtgtccctgacctgcctggtgaagggcttctaccccagcgatatcgccgtggagtgggagagcaacggccagcccgagaacaactacaagaccaccccccctgtgctggacagcgacggcagcttcttcctgtacagcaagctgaccgtggacaagtccaggtggcagcagggaaatgtcttttcttgttctgtcatgcatgaagctctgcacaaccactacacccagaagtccctgagcctgtcccccggcaagtgatagctcgaggatccggacccttccctattcttgtaagtctaaatttactaactgtgctgtttaacttctgatgtttgtatgatatttgagtaattaagagccctacaaaaaaatcaataatgaatggttccaaaataagcatagctgagattaatgattctcagcattagttataaatagaataagctggaaaaccttcacctcccctccaccaccagatctcaatgtctaggcttacccatggagattctgattaactgttctttctatgtagaagaaacttattgggaagaaataatataatggactatgatttaattggtctgttgagaatttagatgaaggggattaagttacaataaagccagaatttaacttgataatctcatttggctaagaataacaaacctaagaaggtttgctattttctacaattttgaagttttccttatgcacaattatttcaccacatgactcatttcacatcttgtttttgatatatgagcatatgagggcaaaatactgaagatgcttatttcaatactcagggaaaattttcttgccaaaaggcaagaattgtataattcattcacttattttattttttttaatttttaaggtctaagaggatttcaaagtgaatgccccctcctcacttttggtaagctttaggagattggaggcagactgatcatttttatagttaatatcttttacatttcatcttcctggataagccccaatagtagcaatttctatcagtataccagcataaagattagttttaaatttattttcagtgattgactgttatttactgacctgaaattatgtatctgttatatttcaaataatgcaaaactgtatatatatggtgttgacagatttgattggttttctttcaattgcctatatccttattattgattgtaatcatttatagaaaaaacaaaataatttcttatacttttatgtaaacctgttagagcttattttaaagatcaactgcattcacatttctaatctagtcattatgagcttcaattgttttatctcacttaaaatttatatattgtcttttaattcatgagtcaaaatacaatctcacagtccagatatgggacttaaaaggggaatagcatatagttttgatattcttaaagatatacatctttttgtgatcatgattcagcagacattttaataaaacaattccaagtgagccgacacttggtcctagaggaatttttataaccttaagataaggcacagcatggtgtttttgtaataagatttcttttatgaaaaagtcacaccaaaattggaaatggggtgagatgaagagttataacatataactaaatggacatttgttctctattccacagaattgactgcgactggaaatatggcaacttttcaatccttgcatcatgctactaagataatttttaaatgagtatacatggaacaaaaaatgaaactttattcctttatttatattatgctttttcatcttaatttgaatttgagtcataaaccatatactttcaaaatgttaattcaacattagcataaaagttcaattttaacttggaaatatcatgaacatatcaaattatgtataaaaataatttctggaattgtgattattatttctttaagaatctatttcctaaccagtcatttcaataaattaacccttaggcatatttaagttttcttgtctttattatatttttaaaaatgaaattggtctctttattgttaacttaaatttatctttgatgttaaaaatagctgtggaaaattaaaattgaatagaattctttgaattgagttccaaaggatatcaaaaagtgagggaaaagatagggtgagcctatgctgcatatgtccttagaaagtcttggtttatacctgttacctaagttaaacaattatacttgttcctttcactctcgaaagtacccagcattggatgttaaattttatagtcatcctagacaaaaaaaaaaaaaaaaacaaacaaccctcaaatgtgatatctgaatcacagctctacagtgtggtagctaagtggtgctgtgtaagttagtctccaagagattccatttctacatttataaacagtcaatttaaggtgttttattgaagttttaatgtgaaaagtgcactatatggtgcatgataggagttcctggttgaatctcatttctgacatcactgacaccagtgcagcaaggactagtgttacaatcagaaggagctgagttgtgtaattttagccattaatgcccaagagactagaacttacacaaagctctaatatccattgtctctgtctgtggagtaattatttcattgccatgaattatctgtctgtcatatcctgcatttttatacatgattcagttcccttcagttcacacaatgacttgtctaatttcatctttcctgcatcctccatgttttcctcacttcaggattaagtgaagccgtacttaggcacaatatttcttatctttaaagaaaaattccatctttgagagttgttattgttcagtcactaggtcatgtccaactctttgtgaccccatgcactgcagcatgccaggcttccctgcccttcgctctctcctggagtttgctcagactcatgtagattgagtcggtgatggtatccaactatctcatcaactgttgtgcccttctcctcctaccctcagtctttaccagcatcagagtctttctcagattcttcaggttattatataacaactatcataaaaggagtatctaaatggctgtgtccattatttcacatgttattctctctttaacttgctccaatcccaattttatccctatgggaactgctttattgaagatcaccaacaacttttattttactaatcgttttgttttacccaacctctcagtgagtgttatgaggtagagttgactatttcttcattttgaaatattacgcttcatttcatttgatatcctaaagctcataaggtgtggtttttctcttaactcactagacacttctttgaagtctctcttctggcattttctccttttccaaaattttaatggttggagtaccctagattttagccttaatttgtttgatgttgttcagttccattctcagctcagagcttccaactgtatgtctccaaacttactcgttttgtaaactccaaactcatgcactcaactgcattcttgacctccacactgaattatctaattaatgtcctaaatctggcatgaccaagcatacatttttgtctgaatccagtccccaacttgctcaaaatttaattaaacgtaattcagttacaaaggcagctgatattgtatgcaatagacctgaatgggaacttcacaaaagaagttatcttaattgtcaataaaaacatgaaaaatactctacatcatcaatcttcagaaaaatgcaaattaaaggtgcctaataatatcatgacacaaccgtcagaatgactgaaatgaaaagaattgtaatacagttcagttcagttcagttactcagtcgtctccaactctttgtgaccccatgaactgcagcatgacagaccttcctgtccatcaccaactcccagagtttactcagactatgtccattgagttgatgatgccatccaaccatctcatcctctgtcgtccccttctcctcctgccctcagtctttcccagcatcagggtcttttccaatgagtcagctcttcgcatcaggtggctaaagtattggagtttcagcttcaacatcagtccttctaattaacacccaggactgatctcttttaggatggactagttggatctccttgcagtccaagggactctcaagagtcttctccaacaccacagttcaaaagcatcaattccttggcactcagctttccttatagtccatgtctcacatccacacatgactattggaaaaaccatagccttgactaggtggacctttgttgacaaagtaatgtctctgctttttaatatgttgtctagattggtcataactttccttccaagaagtaattgtcttttaatttcatggctgcagtcaccatctgcagtgattttggagccccaaaatataaagtcagctgctgtttccactgttgccccatctaccccatctatttgccatgaagtgatgggactggatgccactatcttagttttctgaatgttgagctttaagccagcctttttactctcctctttcactttcatcaagaggctctttagttcctcttcactttctgccataagggtggtgtcatctgcatatctgaggttattgatatttctcttggcaattttgattccagcctgcacttcttccagcccagtgtttctcatgatgtactctgcatataaattaaataagcagagtgacaatatacagccttgacatactctttttcctatttggaaccagtctgttgttccatgtccagttctaactgttgtttcctgacctgcatacaggtttctcaagaggcaagtcaggtggtctggtattctcacctgtttcagaattttccacagtttattgtgatccacacagtcaaaggctttggcatagccaataaagcagaaagagatgtttttctggaactctcttacttttttgatgatccagtggatgttggcaatttgatctctggttcctctgccttttctaaaaccagctttaacatctggaagttcatggttcacgtaatacaaaatgtaatacaaaatgtctgcaaaaacaaaggaatgaaaagtaatgctaaaaaatgttaatatttacagaaatttttatagtagtaaagaattcacctgcaatacaggagaaccgggttagatccctgggttggaagacctcctggagaaggaaatggctacccaatctagtattcttgtctggagaaggcaagaatggacagagaagcccagcgggctatggtccatcgggtcacaaagagtcagaagctaccttgcacacagcaagcacggtgcgcgcgcgtgcacacacacacacacacacacacacagacacacacacactctaaaacatttacccaagcttgtccaatggaaaatcaaaaagccagcaatttaagatgacatcaggtaccactgtccaggtaagcctcagaacacaatgaccagtaagaagcaaagtgccatatgagcaactcgaatttttgcaatgttacctaagagcttccatttttataatgcaaaagaatttcatatggggaaattgtattagataaccctgaatgaggagcaagatatagtcaaagtaagatgctctagtactattttttataagcatgatttgttcagccaaaggttttttcccatatggccaatgaactgaaatatgcagtcctgagatttgcatatatttctagctgaaaccaagtaaataatatcctcaagaaagaaatcaatagaaaagttggatgaagagtacaataaagggaccaaaaatattcagaaataagaactagaggagatattgggaaatccctggtgagtccagtttaggattttgtactttcactgcagttggcatggatataatccctcactggggaactaagatcccataagctgtgttggattgccaaaaaaataaatattaagagatatcattcatagaatattttaaagatattttagagaagaggaaattaaggatgtgagaatttgtattactttttcaagatactaaagctatttagagatagagctgttactaaaaacttcagtttcctaaaaattatttgaagcactgtttaataaattccaaaatatagaggaaggaaaacaaaatactgaggattcatataatgattcagatttagaaacaatataacacagaattagtgaattctgacaaattattaggtaggagtagatagttcagcattactcgtatagatggagtatttaatcctttccatgagattatccaaatataataatttcgtatctatgtgaagtataactattaagattactttataaagtaaatcaagaaccagagaataagaaaaatgttttgtgaaccagcagatactatgaacacataaaactcagaaccctgattcctaagacacacagctaatcctgattattcttcctttacatgtgaccatagaacttcacacaagttcaagatacatttgttgagcacatcagtatcagttcagtcactcagtcatgtccgaatctttgtgaccttgtggactgcagcacgccaggctttcctgtccaccaccaacccctggagcttactcaaactcatgtccattgagtcagtgatcccatccaaccatctcatcctctgtcatcctcttctcctgccttcaatctttcccagacattggagtcttttccaatgagtcagatcttcacattaggtggccaaagtataggagtttcagcttcagcatcaatccttccaatgaatattccttgatgtacccctttcgcagtttggaaccagtctgttgttccatgtccagttctaactgctgcttctggacctgtatacagatttctcaggaggcaggtaaagtggtctggtattcccatctcttgaagaattttccacagtttattgtgatccacacaatcaaaggctttagcgtagtcaataaagcagatgtttttctggaactctcgtgcttttttgatgatccaatggatgttggcaatttgatctctggttcctctgccttttctaaatccagcttgaacatctggaagttcatggtccacgtactgttgaagcctggcttggagaattttgagagttattttgctagcatgtgagatgagtgcaatcatgtgggtgtttgaacatactttgtcattgcttttctttgggattgtggcagtcctgtggccactgctgagttttccaaatttgctgacatattgagtgcagcactttcacagcatcaccttttaagatttgaaatagctcaactggaattccatcacctccactagctttgttcatagtgaggctttctaaggccgtttgactttgcattccagggtgtctggctctaggtgagtgatccgttgacctgcaggtcgagctcgaagcggccgctctagcctcgaggctagaactagtggatctcgagccccagctggttctttccgcctcagaagccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgagctctagatcatcgatgcatggggtcgtgcgctcctttcggtcgggcgctgcgggtcgtggggcgggcgtcaggcaccgggcttgcgggtcatgcaccaggtgcgcggtccttcgggcacctcgacgtcggcggtgacggtgaagccgagccgctcgtagaaggggaggttgcggggcgcggaggtctccaggaaggcgggcaccccggcgcgctcggccgcctccactccggggagcacgacggcgctgcccagacccttgccctggtggtcgggcgagacgccgacggtggccaggaaccacgcgggctccttgggccggtgcggcgccaggaggccttccatctgttgctgcgcggccagccgggaaccgctcaactcggccatgcgcgggccgatctcggcgaacaccgcccccgcttcgacgctctccggcgtggtccagaccgccaccgcggcgccgtcgtccgcgacccacaccttgccgatgtcgagcccgacgcgcgtgaggaagagttcttgcagctcggtgacccgctcgatgtggcggtccggatcgacggtgtggcgcgtggcggggtagtcggcgaacgcggcggcgagggtgcgtacggccctggggacgtcgtcgcgggtggcgaggcgcaccgtgggcttgtactcggtcatggtaagcttcagctgctcgagatctagatggatgcaggtcgaaaggcccggagatgaggaagaggagaacagcgcggcagacgtgcgcttttgaagcgtgcagaatgccgggcctccggaggaccttcgggcgcccgccccgcccctgagcccgcccctgagcccgcccccggacccaccccttcccagcctctgagcccagaaagcgaaggagcaaagctgctattggccgctgccccaaaggcctacccgcttccattgctcagcggtgctgtccatctgcacgagactagtgagacgtgctacttccatttgtcacgtcctgcacgacgcgagctgcggggcgggggggaacttcctgactaggggaggagtagaaggtggcgcgaaggggccaccaaagaacggagccggttggcgcctaccggtggatgtggaatgtgtgcgaggccagaggccacttgtgtagcgccaagtgcccagcggggctgctaaagcgcatgctccagactgccttgggaaaagcgcctcccctacccggtagaatttcgaggtcgaccggccgcgaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtcagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatctctagtcaaggcactatacatcaaatattccttattaacccctttacaaattaaaaagctaaaggtacacaatttttgagcatagttattaatagcagacactctatgcctgtgtggagtaagaaaaaacagtatgttatgattataactgttatgcctacttataaaggttacagaatatttttccataattttcttgtatagcagtgcagctttttcctttgtggtgtaaatagcaaagcaagcaagagttctattactaaacacagcatgactcaaaaaacttagcaattctgaaggaaagtccttggggtcttctacctttctcttcttttttggaggagtagaatgttgagagtcagcagtagcctcatcatcactagatggcatttcttctgagcaaaacaggttttcctcattaaaggcattccaccactgctcccattcatcagttccataggttggaatctaaaatacacaaacaattagaatcagtagtttaacacattatacacttaaaaattttatatttaccttagagctttaaatctctgtaggtagtttgtccaattatgtcacaccacagaagtaaggttccttcacaaagatccggaccaaagcggccatcgtgcctccccactcctgcagttcgggggcatggatgcgcggatagccgctgctggtttcctggatgccgacggatttgcactgccggtagaactccgcgaggtcgtccagcctcaggcagcagctgaaccaactcgcgaggggatcgagcccggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgaagctttttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagccggatctgcaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccaatgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgaggaactttcggcggctttgctgtgcgacaggctcacgtctaaaaggaaataaatcatgggtcataaaaattatcacgttgtcggcgcggcgacggatgttctgtatgcgctgttttccgttggccgttgctgtctggtgatctgccttctaaatctgcacagccgaattgcgcgagcttggttttgctgaaaccgacacacagcaactgaataccagaaagaaaatcactttgcctttctgacatcagaagggcagaaatttgccgttgaacacctggtcaatacgcgttttggtgagcagcaatattgcgcttcgatgagccttggcgttgagattgatacctctgctgcacaaaaggcaatcgaccgagctggaccagcgcattcgtgacaccgtctccttcgaacttattcgcaatggagtgtcattcatcaaggacngcctgatcgcaaatggtgctatccacgcagcggcaatcgaaaaccctcagccggtgaccaatatctacaacatcagccttggtatcctgcgtgatgagccagcgcagaacaaggtaaccgtcagtgccgataagttcaaagttaaacctggtgttgataccaacattgaaacgttgatcgaaaacgcgctgaaaaacgctgctgaatgtgcggcgctggatgtcacaaagcaaatggcagcagacaagaaagcgatggatgaactggcttcctatgtccgcacggccatcatgatggaatgtttccccggtggtgttatctggcagcagtgccgtcgatagtatgcaattgataattattatcatttgcgggtcctttccggcgatccgccttgttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgagctttttggcctctgtcgtttcctttctctgtttttgtccgtggaatgaacaatggaagtcaacaaaaagcagacgtatctagacacgtctgaagctagcttcgaggaactttcggcggctttgctgtgcgacaggctcacgtctaaaaggaaataaatcatgggtcataaaaattatcacgttgtcggcgcggcgacggatgttctgtatgcgctgttttccgttggccgttgctgtctggtgatctgccttctaaatctgcacagccgaattgcgcgagcttggttttgctgaaaccgacacacagcaactgaataccagaaagaaaatcactttgcctttctgacatcagaagggcagaaatttgccgttgaacacctggtcaatacgcgttttggtgagcagcaatattgcgcttcgatgagccttggcgttgagattgatacctctgctgcacaaaaggcaatcgaccgagctggaccagcgcattcgtgacaccgtctccttcgaacttattcgcaatggagtgtcattcatcaaggacngcctgatcgcaaatggtgctatccacgcagcggcaatcgaaaaccctcagccggtgaccaatatctacaacatcagccttggtatcctgcgtgatgagccagcgcagaacaaggtaaccgtcagtgccgataagttcaaagttaaacctggtgttgataccaacattgaaacgttgatcgaaaacgcgctgaaaaacgctgctgaatgtgcggcgctggatgtcacaaagcaaatggcagcagacaagaaagcgatggatgaactggcttcctatgtccgcacggccatcatgatggaatgtttccccggtggtgttatctggcagcagtgccgtcgatagtatgcaattgataattattatcatttgcgggtcctttccggcgatccgccttgttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgagctttttggcctctgtcgtttcctttctctgtttttgtccgtggaatgaacaatggaagtcaacaaaaagcagagcttatcgatgataagcggtcaaacatgagaattcgtcgg

(19) United States
(12) Patent Application Publication
(10) Pub. No.: US 2016/0002330 A1
(43) Pub. Date: Jan. 7, 2016

(54) CETUXIMAB WITH MODIFIED GLYCOSYLATION AND USES THEREOF

(71) Applicant: LABORATOIRE FRANCAIS DU FRACTIONNEMENT ET DES BIOTECHNOLOGIES, Les Ulis (FR)

(72) Inventor: Harry M. MEADE, Newton, MA (US)

(21) Appl. No.: 14/767,089

(22) PCT Filed: Feb. 13, 2014
(86) PCT No.: PCT/IB2014/000867

§ 371 Date: Aug. 11, 2015

Related U.S. Application Data
(60) Provisional application No. 61/764,446, filed on Feb. 13, 2013.

(51) Int. Cl. C07K 16/28; A61K 31/513; A61K 31/4745; A61K 31/522; A61K 39/395; A01K 67/027

(52) U.S. Cl. CPC C07K 16/28; A61K 39/39533; A01K 67/0278; A61K 31/4745; A61K 31/522; A61K 31/513; C07K 2317/24; C07K 2317/21; C07K 2317/14; A61K 2039/505

(57) ABSTRACT

In one aspect, the disclosure relates to antibodies with altered glycosylation patterns, methods of production of said antibodies, and methods of use thereof. In some embodiments, the antibody is cetuximab.

### RELATED APPLICATIONS

This application claims the benefit under 35 U.S.C. §119(e) of U.S. Provisional Application Ser. No. 61/764,446, entitled “Cetuximab with Modified Glycosylation and Uses Thereof,” filed on Feb. 13, 2013, the entire disclosure of which is incorporated by reference herein in its entirety.

### FIELD OF THE INVENTION

The present invention relates in part to antibodies with altered glycosylation patterns, methods of their production, and methods of use.

### BACKGROUND OF THE INVENTION

Erbitux® (cetuximab) is a recombinant chimeric monoclonal antibody that binds to the extracellular domain of the human epidermal growth factor receptor (EGFR) and is used in the clinic to treat certain forms of cancer. Unfortunately, some patients have experienced an allergic response, in some cases anaphylactic shock and death, when treated with Erbitux®. This allergic response has been shown to be caused by IgE that recognizes the galactose-alpha-1,3-galactose glycosylation molecule present on the commercial form of cetuximab, Erbitux® (C. Chung, et al. Cetuximab-induced anaphylaxis and IgE specific for galactose-alpha-1,3-galactose. N Engl J Med (2008);358:1109-17). Addition of galactose-alpha-1,3-galactose is thought to be a result of the mammalian (murine myeloma) cell culture system used to commercially produce Erbitux®. Production of proteins in Chinese hamster ovary cells can also result in galactose-alpha-1,3-galactose moieties on the proteins (Bosques et al., Nat. Biotechn. 28: 1153-1156, 2010).

### SUMMARY OF INVENTION

It has been found that antibodies produced in mammalian mammary epithelial cells, such as in the mammalian mammary epithelial cells of a transgenic animal engineered to secrete antibodies in its milk, have no detectable galactose-alpha-1,3-galatose moieties. Accordingly, in aspect, the disclosure provides antibodies and compositions thereof that have no detectable galactose-alpha-1,3-galatose moieties. Also provided are methods of producing such antibodies as well as methods of their use.

In one aspect, the disclosure provides a glycosylated antibody comprising

(a) a heavy chain comprising SEQ ID NO:1;

(b) a light chain comprising SEQ ID NO:2;

wherein the glycosylated antibody has fewer galactose-alpha-1,3-galactose moieties than the glycosylated antibody produced in non-mammary cell culture.

In some of the embodiments of the glycosylated antibodies provided herein, the non-mammary cell culture is mouse myeloma cell culture. In some of the embodiments of the glycosylated antibodies provided herein, the fewer galactose-alpha-1,3-galactose moieties are located in the variable region of the heavy chain. In some of the embodiments of the glycosylated antibodies provided herein, the fewer galactose-alpha-1,3-galactose moieties are located at position 107 of the heavy chain as shown in SEQ ID NO:1. In some of the embodiments of the glycosylated antibodies provided herein, the glycosylated antibody lacks galactose-alpha-1,3-galactose moieties. In some of the embodiments of the glycosylated antibodies provided herein, the glycosylated antibody is produced in mammary gland epithelial cells. In some of the embodiments of the glycosylated antibodies provided herein, the glycosylated antibody is produced in the mammary gland epithelial cells of a non-human transgenic mammal. In some embodiments, the non-human mammal is a goat, sheep, bison, camel, cow, pig, rabbit, buffalo, horse, rat, mouse or llama. In some embodiments, the non-human mammal is a goat. In some of the embodiments of the glycosylated antibodies provided herein, the antibody is chimeric, humanized or a fully human antibody. In some of the embodiments of the glycosylated antibodies provided herein, the antibody is cetuximab.

In one aspect, the disclosure provides a composition comprising any of the glycosylated antibodies provided herein, and a pharmaceutically-acceptable carrier.

In one aspect, the disclosure provides a composition comprising a population of glycosylated antibodies, wherein the glycosylated antibody comprises:

(a) a heavy chain comprising SEQ ID NO:1;

(b) a light chain comprising SEQ ID NO:2; and

wherein the population of antibodies has fewer galactose-alpha-1,3-galactose moieties than the population of glycosylated antibodies produced in non-mammary cell culture.

In some of the embodiments of the compositions provided herein, the non-mammary cell culture is mouse myeloma cell culture. In some of the embodiments of the compositions provided herein, the fewer galactose-alpha-1,3-galactose moieties are located in the variable region of the heavy chain. In some of the embodiments of the compositions provided herein, the fewer galactose-alpha-1,3-galactose moieties are located at position 107 of the heavy chain as shown in SEQ ID NO:1. In some of the embodiments of the compositions provided herein, the glycosylated antibodies lack galactose-alpha-1,3-galactose moieties. In some of the embodiments of the compositions provided herein, the population of glycosylated antibodies is produced in mammary gland epithelial cells. In some of the embodiments of the compositions provided herein, the population of glycosylated antibodies is produced in the mammary gland epithelial cells of a non-human transgenic mammal. In some embodiments, the non-human mammal is a goat, sheep, bison, camel, cow, pig, rabbit, buffalo, horse, rat, mouse or llama.

In some embodiments, the non-human mammal is a goat.

In some of the embodiments of the compositions provided herein, the glycosylated antibodies in the population are chimeric, humanized or fully human antibodies. In some of the embodiments of the compositions provided herein, the glycosylated antibodies in the population are cetuximab antibodies. In some of the embodiments of the compositions provided herein, the composition further comprises milk. In some of the embodiments of the compositions provided herein, the composition further comprises a pharmaceutically acceptable carrier.

In one aspect, the disclosure provides a composition comprising monoclonal antibodies, wherein the monoclonal antibodies comprise:

(a) a heavy chain comprising SEQ ID NO:1;

(b) a light chain comprising SEQ ID NO:2; and

wherein the composition is produced in a mammary gland of a transgenic goat.

In one aspect, the disclosure provides a composition comprising monoclonal antibodies, wherein the monoclonal antibodies comprise:

(a) a heavy chain comprising SEQ ID NO:1;

(b) a light chain comprising SEQ ID NO:2; and

wherein the monoclonal antibodies lack galactose-alpha-1,3-galactose.

In one aspect, the disclosure provides a method comprising producing a population of glycosylated antibodies in mammary gland epithelial cells such that the population of glycosylated antibodies produced comprises fewer galactose-alpha-1,3-galactose moieties than the population of glycosylated antibodies produced in non-mammary cell culture, and

wherein the glycosylated antibodies comprise a heavy chain comprising SEQ ID NO:1 and a light chain comprising SEQ ID NO:2.

In some of the embodiments of the methods provided herein, the non-mammary cell culture is mouse myeloma cell culture. In some of the embodiments of the methods provided herein, the population of glycosylated antibodies lacks galactose-alpha-1,3-galactose. In some of the embodiments of the methods provided herein, the method further comprises collecting the population of glycosylated antibodies. In some of the embodiments of the methods provided herein, the producing occurs in vitro. In some of the embodiments of the methods provided herein, the producing occurs in vivo. In some of the embodiments of the methods provided herein, the producing occurs in a non-human transgenic mammal. In some embodiments, the non-human mammal is a goat, sheep, bison, camel, cow, pig, rabbit, buffalo, horse, rat, mouse or llama. In some embodiments, the non-human mammal is a goat.

In some of the embodiments of the methods provided herein, the method further comprising purifying the population of glycosylated antibodies. In some of the embodiments of the methods provided herein, the method further comprises comparing the number of galactose-alpha-1,3-galactose moieties present in the population of glycosylated antibodies to the number of galactose-alpha-1,3-galactose moieties present in a population of glycosylated antibodies produced in non-mammary cell culture.

In one aspect, the disclosure provides a population of glycosylated antibodies produced by any of the methods provided herein.

In one aspect, the disclosure provides mammary gland epithelial cells that produce any of the antibodies provided herein. In some embodiments of the mammary gland epithelial cells provided herein, the mammary gland epithelial cells comprise a nucleic acid comprising SEQ ID NO:3 and a nucleic acid comprising SEQ ID NO:4. In some embodiments of the mammary gland epithelial cells provided herein, the nucleic acid comprising SEQ ID NO:3 and the nucleic acid comprising SEQ ID NO:4 are connected.

In one aspect, the disclosure provides a transgenic non-human mammal comprising any of the mammary gland epithelial cells disclosed herein. In some embodiments, the non-human mammal is a goat.

In one aspect, the disclosure provides mammary gland epithelial cells that produce any of the population of antibodies disclosed herein. In some embodiments of the mammary gland epithelial cells provided herein, the mammary gland epithelial cells comprise a nucleic acid comprising SEQ ID NO: 3 and a nucleic acid comprising SEQ ID NO: 4. In some embodiments of the mammary gland epithelial cells provided herein, the nucleic acid comprising SEQ ID NO: 3 and the nucleic acid comprising SEQ ID NO: 4 are connected.

In one aspect, the disclosure provides a transgenic non-human mammal comprising any of the mammary gland epithelial cells disclosed herein.

In one aspect, the disclosure provides a method comprising administering an effective amount of any of the antibodies provided herein to a subject in need thereof.

In one aspect, the disclosure provides a method comprising administering an effective amount of any of the compositions provided herein to a subject in need thereof.

In some embodiments, the subject has cancer. In some embodiments, the cancer is head and neck cancer or colorectal cancer. In some embodiments of the methods provided herein, the method further comprises administering at least one additional therapeutic agent.

In some embodiments, the at least one additional therapeutic agent is irinotecan, leucovorin calcium, fluorouracil, or a combination thereof.

### BRIEF DESCRIPTION OF THE DRAWINGS

The drawings are first described. It is to be understood that the drawings are exemplary and not required for enablement of the invention.

FIG. 1 shows the amino acid sequence of the heavy chain (HC) of cetuximab (SEQ ID NO:1).

FIG. 2 shows the amino acid sequence of the light chain (LC) of cetuximab (SEQ ID NO:2).

FIG. 3 shows the nucleic acid sequence of the heavy chain (HC) of cetuximab (SEQ ID NO:3).

FIG. 4 shows the nucleic acid sequence of the light chain (LC) of cetuximab (SEQ ID NO:4).

FIG. 5 shows the plasmid BC 2553 LC cetuximab (light chain) (A) and the nucleic acid sequence of the plasmid (SEQ ID NO:5) (B).

FIG. 6 shows the plasmid BC 2554 HC cetuximab (heavy chain) (A) and the nucleic acid sequence of the plasmid (SEQ ID NO:6) (B).

FIG. 7 shows the plasmid BC 2584 puromycin beta casein vector carrying heavy chain (A) and the nucleic acid sequence of the plasmid (SEQ ID NO:7) (B).

FIG. 8 shows a Western Blot with the levels of expression of cetuximab in the milk of two mouse lines (#15 and #27).

FIG. 9 shows a Western Blot used to detect levels of alpha-1,3-gal in milk-produced cetuximab and commercially produced cetuximab (Erbitux®).

FIG. 10 shows a Western Blot with of cetuximab produced in the milk of goats #1 and #2.

FIG. 11 shows the purification of cetuximab produced in the milk of goats.

FIG. 12 is a western blot depicting anti-alpha 1,3 galactose monoclonal antibody staining on cetuximab produced in goats (lane 2, goat #1, and lane 3, goat #2), and the commercially product Erbitux®.

FIG. 13 is a mass spectrum (LC/MS) of purified Erbitux®, purified cetuximab produced in the milk of goat #1, and purified cetuximab produced in the milk of goat #2.

### DETAILED DESCRIPTION OF INVENTION

Erbitux® (cetuximab) is a human/mouse chimeric recombinant antibody that binds to the extracellular domain of the human epidermal growth factor receptor (EGFR) and is used in the clinic to treat cancer. A study of the structure of Erbitux® showed that one N-linked glycosylation site present in the framework 3 region of the heavy chain is occupied by galactose-alpha-1,3-galactose (also referred to herein as “gal-alpha-1,3-gal” or “alpha-1,3-gal”), see Jun Qian, et al.: Structural characterization of N-linked oligosaccharides on monoclonal antibody cetuximab by the combination of orthogonal matrix-assisted laser desorption/ionization hybrid quadrupole-quadrupole time-of-flight tandem mass spectrometry and sequential enzymatic digestion. (Analytical Biochemistry 364 (2007) 8-18). The presence of this gal-alpha-1,3-gal is thought to be due to the fact that commercial Erbitux® is produced in mouse myeloma cells, which are known to add gal-alpha-1,3-gal on N-linked glycosylation sites.

This gal-alpha-1,3-gal glycosylation has been shown to give rise to an allergic response in some patients treated with Erbitux® (Chung, et al. Cetuximab-induced anaphylaxis and IgE specific for galactose-alpha-1,3-galactose. N Engl J Med (2008);358:1109-17). These patients were shown to have pre-existing IgE antibodies to gal-alpha-1,3-gal, which gave rise to a hypersensitive reaction upon treatment with the antibody.

Additionally, galactose-alpha-1,3-galactose has been shown to cause allergic responses in other contexts. For instance, gal-alpha-1,3-gal is present on red meat and people who experience an allergic reaction (e.g., anaphylaxis) after eating red meat were found to have IgE antibodies specific to gal-alpha-1,3-gal (Commins et al., Delayed anaphylaxis, angioedema, or urticaria after consumption of red meat in patients with IgE antibodies specific for galactose-alpha-1,3-galactose. J Allergy Clin Immunol. 2009 February123(2):426-33; and Commins and Platts-Mills, Anaphylaxis syndromes related to a new mammalian cross-reactive carbohydrate determinant. J Allergy Clin Immunol. 2009 October; 124(4): 652-657.). In another instance, it was shown that gal-alpha-1,3-gal present on cat IgA, another allergen, can be bound by IgE in humans (Gronlund et al. The carbohydrate galactose-alpha-1,3-galactose is a major IgE-binding epitope on cat IgA. J Allergy Clin Immunol. 2009 May; 123(5): 1189-91). Hence, the presence of galactose-alpha-1,3-galactose poses potential problems in the development of antibodies and other glycoproteins for therapeutic use in humans.

Production of recombinant antibodies is often performed in cell culture using well-established cell lines like NSo and Sp2/0. These cell lines have been shown to add galactose to recombinant antibodies in an alpha(1-3) linkage to form gal-alpha-1,3-gal (Chung, et al. Cetuximab-induced anaphylaxis and IgE specific for galactose-alpha-1,3-galactose. N Engl J Med (2008);358:1109-17). In humans and higher primates the gene that produces gal-alpha-1,3-gal through addition of galactose in an alpha(1-3) linkage (alpha-1,3-galactosyltransferase) is non-functional, and humans and higher primates therefore generally do not produce gal-alpha-1,3-gal. As a result, gal-alpha-1,3-gal negative animals, such as humans, have the potential to recognize gal-alpha-1,3-gal as “non-self” and can generate IgE and IgG antibodies specific for this oligosaccharide, potentially giving rise to the allergic reactions described above. Thus, a need exists for methods to produce recombinant therapeutic antibodies that lack gal-alpha-1,3-gal moieties.

The ability to modify animal genomes through transgenic technology offers alternatives for the manufacture of recombinant antibodies with modified glycosylation patterns. The production of human recombinant pharmaceuticals in the milk of transgenic farm animals solves many of the problems associated with microbial bioreactors (e.g., lack of proper post-translational modifications, improper protein folding, high purification costs) or animal cell bioreactors (e.g., high capital costs, expensive culture media, low yields).

Surprisingly, as disclosed herein, cetuximab produced in the milk of mice and goats through expression of the heavy and light chains of cetuximab in mammary epithelial cells was found to have no detectable gal-alpha-1,3-gal (as determined by LC/MS and Western blot analysis). This is surprising, at least, because commercially produced cetuximab (Erbitux®), which is produced in mouse myeloma cells contains gal-alpha-1,3-gal moieties. In addition, goat serum proteins also show gal-alpha-1,3-gal moieties.

Accordingly, aspects of the disclosure relate to glycosylated antibodies or populations of antibodies that have altered glycosylation patterns, specifically fewer gal-alpha-1,3-gal moieties compared to glycosylated antibodies produced in cell culture. In some embodiments, the antibody is cetuximab. Other aspects of the disclosure provide methods for producing glycosylated antibody or populations of antibodies that comprise fewer gal-alpha-1,3-gal moieties than glycosylated antibodies produced in cell culture. Yet another aspect of the disclosure relates to mammalian mammary epithelial cells or transgenic animals capable of producing populations of antibodies that have altered glycosylation patterns, specifically fewer gal-alpha-1,3-gal moieties compared to glycosylated antibodies produced in cell culture. In some embodiments, the glycosylated antibodies provided herein do not have any detectable level of gal-alpha-1,3-gal moieties.

### Antibodies and Antigen-binding Fragments Thereof

As used herein, the term “antibody” can refer to a glycoprotein comprising at least two heavy (H) chains and two light (L) chains. Each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. In some embodiments, the antigen is the extracellular domain of the human epidermal growth factor receptor (EGFR). The constant regions of the antibodies may mediate the binding of the immunoglobulin to host tissues or factors, including various cells of the immune system (e.g., effector cells) and the first component (Clq) of the classical complement system. Formation of a mature functional antibody molecule can be accomplished when two proteins are expressed in stoichiometric quantities and self-assemble with the proper configuration.

The term “antibodies” is also meant to encompass antigen-binding fragments thereof. Methods for making antibodies and antigen-binding fragments are well known in the art (see, e.g. Sambrook et al, “Molecular Cloning: A Laboratory Manual” (2nd Ed.), Cold Spring Harbor Laboratory Press (1989); Lewin, “Genes IV”, Oxford University Press, New York, (1990), and Roitt et al., “Immunology” (2nd Ed.), Gower Medical Publishing, London, New York (1989), WO2006/040153, WO2006/122786, and WO2003/002609). As used herein, an “antigen-binding fragment” of an antibody refers to one or more portions of an antibody that retain the ability to specifically bind to an antigen, e.g. the extracellular domain of EGFR. It has been shown that the antigen-binding function of an antibody can be performed by fragments of a full-length antibody. Examples of binding fragments encompassed within the term “antigen-binding fragment” of an antibody include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546) which consists of a VH domain; and (vi) an isolated complementarity determining region (CDR). Furthermore, although the two domains of the Fv fragment, VL and VH, are encoded by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. These antibody fragments are obtained using conventional procedures, such as proteolytic fragmentation procedures, as described in J. Goding, Monoclonal Antibodies: Principles and Practice, pp 98-118 (N.Y. Academic Press 1983), which is hereby incorporated by reference as well as by other techniques known to those with skill in the art. The fragments are screened for utility in the same manner as are intact antibodies.

In some embodiments, the antibodies are of the isotype IgG, IgA or IgD. In further embodiments, the antibodies are selected from the group consisting of IgG1, IgG2, IgG3, IgG4, IgM, IgA1, IgA2, IgAsec, IgD, IgE or has immunoglobulin constant and/or variable domain of IgG1, IgG2, IgG3, IgG4, IgM, IgA1, IgA2, IgAsec, IgD or IgE. In other embodiments, the antibodies are bispecific or multispecific antibodies. According to an alternative embodiment, the antibodies of the present disclosure can be modified to be in the form of a bispecific antibody, or a multispecific antibody. The term “bispecific antibody” is intended to include any agent, e.g., a protein, peptide, or protein or peptide complex, which has two different binding specificities which bind to, or interact with (a) a cell surface antigen and (b) an Fc receptor on the surface of an effector cell. The term “multispecific antibody” is intended to include any agent, e.g., a protein, peptide, or protein or peptide complex, which has more than two different binding specificities which bind to, or interact with (a) a cell surface antigen, (b) an Fc receptor on the surface of an effector cell, and (c) at least one other component. Accordingly, the disclosure includes, but is not limited to, bispecific, trispecific, tetraspecific, and other multispecific antibodies which are directed to cell surface antigens, and to Fc receptors on effector cells. The term “bispecific antibodies” further includes diabodies. Diabodies are bivalent, bispecific antibodies in which the VH and VL domains are expressed on a single polypeptide chain, but using a linker that is too short to allow for pairing between the two domains on the same chain, thereby forcing the domains to pair with complementary domains of another chain and creating two antigen-binding sites (see e.g., Holliger, P., et al. (1993) Proc. Natl. Acad. Sci. USA 90:6444-6448; Poijak, R. J., et al. (1994) Structure 2:1121-1123).

The term “antibodies” also encompasses different types of antibodies, e.g., recombinant antibodies, monoclonal antibodies, humanized antibodies or chimeric antibodies, or a mixture of these.

In some embodiments, the antibodies are recombinant antibodies. The term “recombinant antibody”, as used herein, is intended to include antibodies that are prepared, expressed, created or isolated by recombinant means, such as antibodies isolated from an animal that is transgenic for another species' immunoglobulin genes, antibodies expressed using a recombinant expression vector transfected into a host cell, antibodies isolated from a recombinant, combinatorial antibody library, or antibodies prepared, expressed, created or isolated by any other means that involves splicing of immunoglobulin gene sequences to other DNA sequences.

In yet other embodiments, the antibodies can be chimeric or humanized antibodies. As used herein, the term “chimeric antibody” refers to an antibody that combines parts of a non-human (e.g., mouse, rat, rabbit) antibody with parts of a human antibody. As used herein, the term “humanized antibody” refers to an antibody that retains only the antigen-binding CDRs from the parent antibody in association with human framework regions (see, Waldmann, 1991, Science 252:1657). Such chimeric or humanized antibodies retaining binding specificity of the murine antibody are expected to have reduced immunogenicity when administered in vivo for diagnostic, prophylactic or therapeutic applications according to the disclosure. In some embodiments, the antibody is cetuximab.

In certain embodiments, the antibodies are human antibodies. The term “human antibody”, as used herein, is intended to include antibodies having variable and constant regions derived from human germline immunoglobulin sequences. The human antibodies of the disclosure may include amino acid residues not encoded by human germline immunoglobulin sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences (referred to herein as “humanized antibodies”). Human antibodies are generated using transgenic mice carrying parts of the human immune system rather than the mouse system. Fully human monoclonal antibodies also can be prepared by immunizing mice transgenic for large portions of human immunoglobulin heavy and light chain loci. See, e.g., U.S. Pat. Nos. 5,591,669, 5,598,369, 5,545,806, 5,545,807, 6,150,584, and references cited therein, the contents of which are incorporated herein by reference. These animals have been genetically modified such that there is a functional deletion in the production of endogenous (e.g., murine) antibodies. The animals are further modified to contain all or a portion of the human germ-line immunoglobulin gene locus such that immunization of these animals results in the production of fully human antibodies to the antigen of interest. Following immunization of these mice (e.g., XenoMouse (Abgenix), HuMAb mice (Medarex/GenPharm)), monoclonal antibodies are prepared according to standard hybridoma technology. These monoclonal antibodies have human immunoglobulin amino acid sequences and therefore will not provoke human anti-mouse antibody (HAMA) responses when administered to humans. The human antibodies, like any of the antibodies provided herein can be monoclonal antibodies.

In some embodiments, the antibody is a full-length antibody. In some embodiments the full-length antibody comprises a heavy chain and a light chain. In some embodiments, the heavy chain comprises SEQ ID NO:1 and the light chain comprises SEQ ID NO:2. In some embodiments, the heavy chain consists of SEQ ID NO:1 and the light chain consists of SEQ ID NO:2. In some embodiments, the antibody is cetuximab.

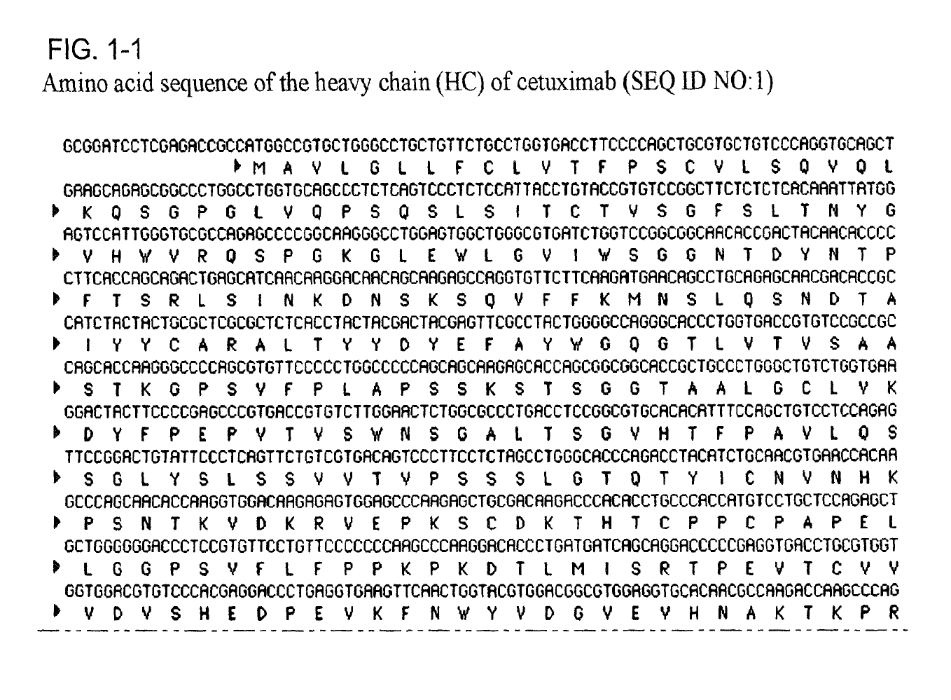

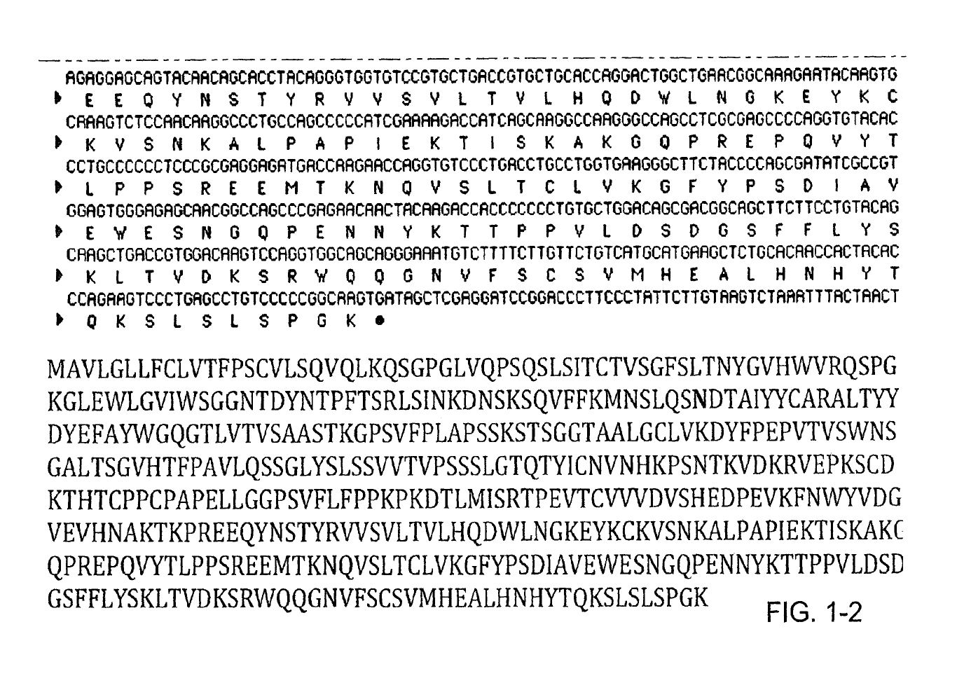

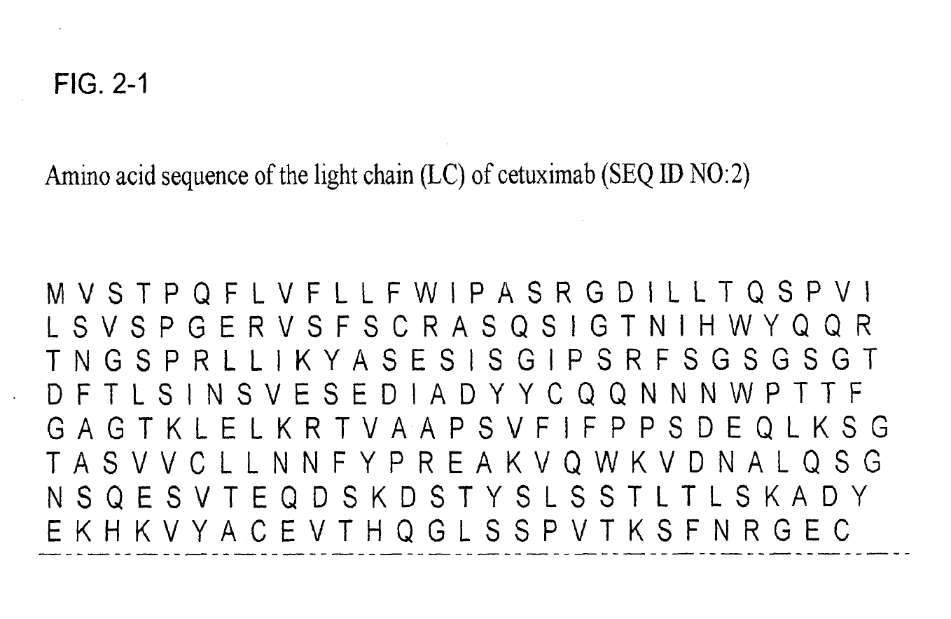

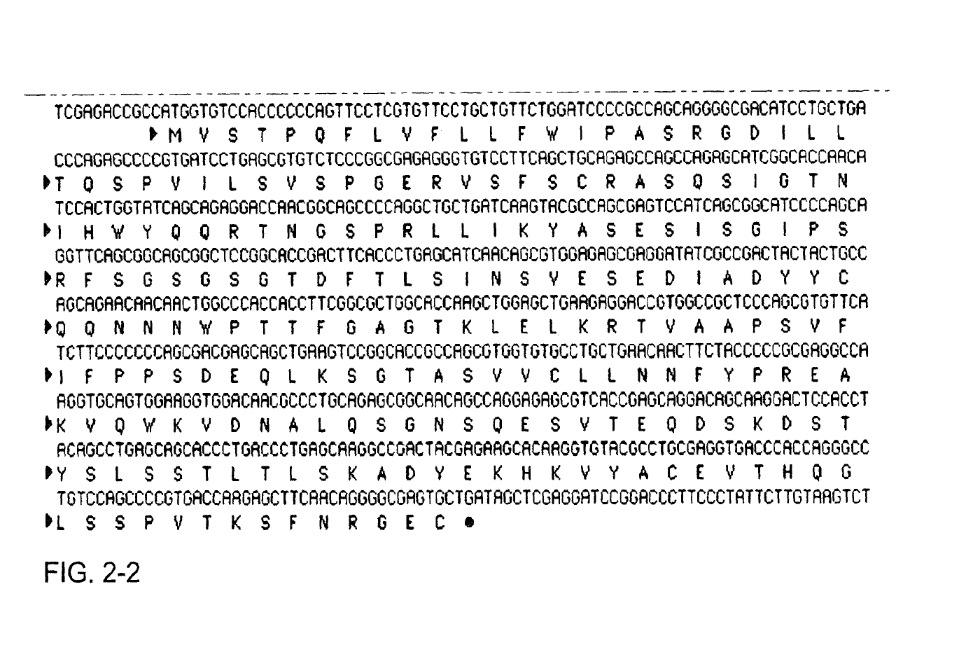

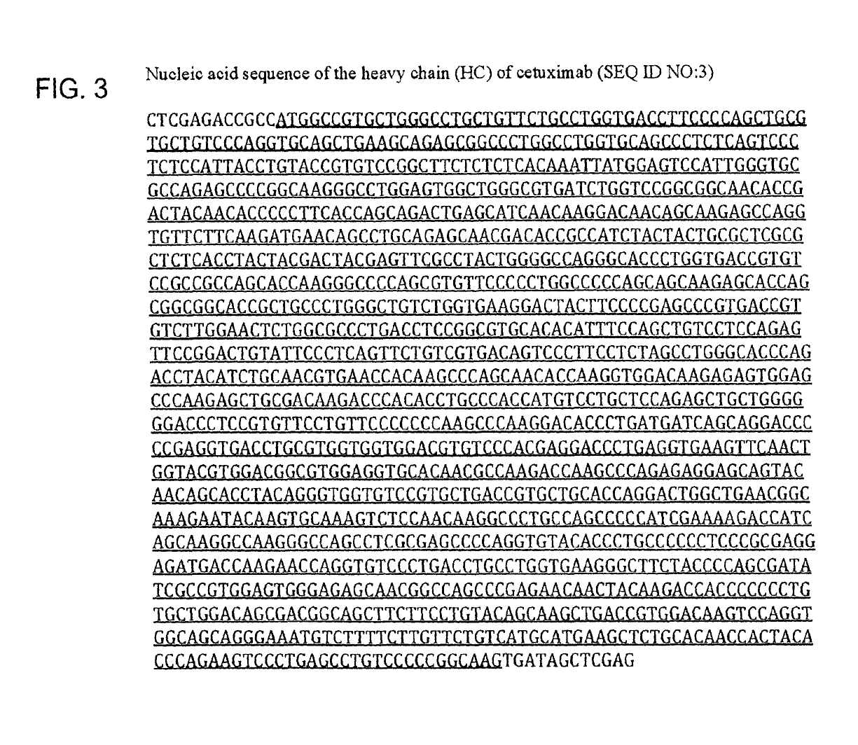

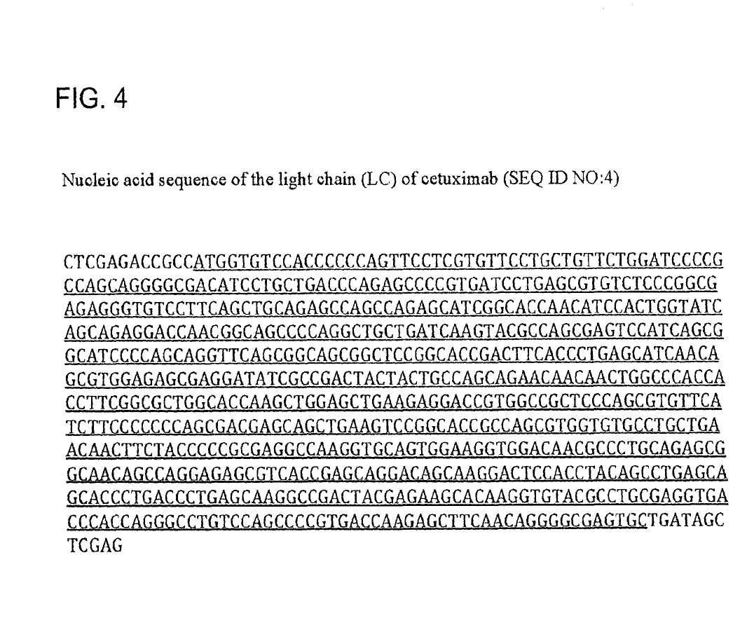

In some embodiments, the antibody is a glycosylated antibody. Glycosylated antibodies are described in greater detail herein. In some embodiments, the glycosylated antibody comprises a heavy chain comprising SEQ ID NO:1 and a light chain comprising SEQ ID NO:2. In some embodiments, the glycosylated antibody comprises a heavy chain that consists of SEQ ID NO:1 and a light chain that consists of SEQ ID NO:2. In some embodiments, the glycosylated antibody has fewer galactose-alpha-1,3-galactose moieties than the glycosylated antibody produced in non-mammary cell culture. In some embodiments, the fewer galactose-alpha-1,3-galactose moieties are located in the variable region of the heavy chain of the glycosylated antibody. In one embodiment, the fewer galactose-alpha-1,3-galactose moieties are located at position 107 in the heavy chains as shown in SEQ ID NO:1 (Highlighted in FIG. 1).

### Glycosylation

Glycosylation is important for the correct folding, targeting, bioactivity and clearance of therapeutic glycoproteins. Glycosylation is a post-translational modification that can produce a variety of final protein forms in the natural state. Glycosylation patterns differ between different animals, e.g. between mice and humans. Certain alterations in carbohydrate structure also can affect antibody function, tolerability, or therapeutic effectiveness. For example, as described above, certain sugar moieties, such as gal-alpha-1,3-gal, can cause allergic reactions in humans.

Most animals, aside from humans and higher primates incorporate alpha-1,3-gal on N-linked sugars. For instance, CHO (Chinese Hamster Ovary) cell line used for the production of therapeutic proteins puts low level of alpha-1,3-gal on some sites. Furthermore, the mouse myeloma cell line used for the production of the therapeutic antibody cetuximab also incorporates alpha-1,3-gal moieties.

Surprisingly, as disclosed herein, recombinant cetuximab produced in the mammary glands of both transgenic mice and goats did not have detectable gal-alpha-1,3-gal. As also noted above, the result is surprising, at least because cetuximab that is produced in mouse myeloma cells (Erbitux®) is known to contain gal-alpha-1,3-gal sugars (Jun Qian, et al. Structural characterization of N-linked oligosaccharides on monoclonal antibody cetuximab by the combination of orthogonal matrix-assisted laser desorption/ionization hybrid quadrupole-quadrupole time-of-flight tandem mass spectrometry and sequential enzymatic digestion. Analytical Biochemistry 364 (2007) 8-18).

Thus, in some aspects, the disclosure provides a glycosylated antibody or populations of glycosylated antibodies with altered glycosylation patterns. In some embodiments, the disclosure provides a glycosylated antibody or populations of glycosylated antibodies that comprise fewer gal-alpha-1,3-gal moieties than a glycosylated antibody or populations of glycosylated antibodies produced in cell culture. In some embodiments, the cell culture resulting in the production of higher levels gal-alpha-1,3-gal moieties is a non-mammary cell culture. As described herein, “non-mammary cell culture” means a cell culture system in which the cells used are not of mammary tissue origin. In some embodiments, the non-mammary cell culture is mammalian cell culture. In some embodiments, the non-mammary cell culture is mouse cell culture. In some embodiments, the non-mammary cell culture is mouse myeloma cell culture.

In some embodiments, the disclosure provides a glycosylated antibody or populations of glycosylated antibodies that lack gal-alpha-1,3-gal moieties. As used herein, “a glycosylated antibody or populations of glycosylated antibodies that lack gal-alpha-1,3-gal moieties” describes either an antibody or populations of antibodies that contain no gal-alpha-1,3-gal moieties within the entire antibody, or antibodies that do not have detectable gal-alpha-1,3-gal at a specified location within the antibody. Methods for detecting gal-alpha-1,3-gal are well-known in the art and are also described herein. In some embodiments, the glycosylated antibody or populations of glycosylated antibodies of the disclosure comprise a heavy chain comprising SEQ ID NO:1 and a light chain comprising SEQ ID NO:2. In some embodiments, the glycosylated antibody or populations of glycosylated antibodies disclosed herein are cetuximab antibodies.

In other aspects, the disclosure provides methods for producing glycosylated antibodies with altered glycosylation patterns. In some embodiments, the disclosure provides methods for producing glycosylated antibodies that comprise fewer gal-alpha-1,3-gal moieties than glycosylated antibodies produced in cell culture. In some embodiments, the fewer galactose-alpha-1,3-galactose moieties are located in the variable region of the heavy chain of the glycosylated antibody. In one embodiment, the fewer galactose-alpha-1,3-galactose moieties are located at amino acid position 107 in SEQ ID NO:1. In some embodiments, the cell culture is non-mammary cell culture. In some embodiments, the non-mammary cell culture is mammalian cell culture. In some embodiments, the non-mammary cell culture is mouse cell culture. In some embodiments, the non-mammary cell culture is mouse myeloma cell culture. In some embodiments, the disclosure provides methods for producing glycosylated antibodies that lack gal-alpha-1,3-gal moieties. In some embodiments, the methods above comprise production of glycosylated antibodies comprising a heavy chain comprising SEQ ID NO:1 and a light chain comprising SEQ ID NO:2. In some embodiments, the methods above comprise production of cetuximab.

In some embodiments, a glycosylated antibody or populations of glycosylated antibodies comprise fewer gal-alpha-1,3-gal moieties than a glycosylated antibody or population or glycosylated antibodies produced in cell culture, e.g. non-mammary cell culture. In some embodiments, a glycosylated antibody or populations of glycosylated antibodies comprise at least 10%, at least 20%, at least 30%, at least 40%, at least 50%, at least 60%, at least 70%, at least 80%, at least 90%, or 100% fewer gal-alpha-1,3-gal moieties than a glycosylated antibody or population of glycosylated antibodies produced in cell culture, e.g. non-mammary cell culture. The glycosylation pattern, e.g., the number of gal-alpha-1,3-gal moieties, can be determined by many methods known in the art, e.g., liquid chromatography-mass spectrometry (LC/MS) or tandem mass spectrometry. For example, methods of analyzing carbohydrates on proteins have been described in U.S. Patent Applications US 2006/0057638 and US 2006/0127950. The methods of analyzing carbohydrates on proteins are incorporated herein by reference. Thus, the quantity of gal-alpha-1,3-gal moieties in an antibody or population of antibodies can be measured and compared to another antibody or population of antibodies, determining whether there are fewer gal-alpha-1,3-gal moieties in one population versus the other.

In another aspect, the disclosure provides compositions of glycosylated antibodies or populations of glycosylated antibodies. In one aspect, the disclosure provides a composition comprising a population of glycosylated antibodies, wherein the glycosylated antibodies comprise:

(a) a heavy chain comprising SEQ ID NO:1;

(b) a light chain comprising SEQ ID NO:2; and

wherein the population of antibodies has fewer galactose-alpha-1,3-galactose moieties than the population of glycosylated antibodies produced in non-mammary cell culture.

In another aspect, the disclosure provides a composition comprising monoclonal antibodies, wherein the monoclonal antibodies comprise:

(a) a heavy chain comprising SEQ ID NO:1;

(b) a light chain comprising SEQ ID NO:2; and

wherein the composition is produced in a mammary gland of a transgenic goat.

In yet another aspect, the disclosure provides a composition comprising monoclonal antibodies, wherein the monoclonal antibodies comprise:

(a) a heavy chain comprising SEQ ID NO:1;

(b) a light chain comprising SEQ ID NO:2; and

wherein the monoclonal antibodies lack galactose-alpha-1,3-galactose.

Compositions of antibodies or populations of antibodies as described above may have any further limitations as described herein. For example, in some embodiments, compositions are produced in the mammary gland of a transgenic non-human animal, e.g., a goat. In some embodiments, the composition further comprises milk. In other embodiments, the composition further comprises a pharmaceutically acceptable carrier. In some embodiments, the antibodies or populations of antibodies comprise fewer gal-alpha-1,3-gal moieties than glycosylated antibodies produced in cell culture, e.g. non-mammary cell culture. In other embodiments, the antibodies or populations of antibodies lack gal-alpha-1,3-gal moieties.

### Mammalian Mammary Gland Epithelial Cells and Transgenic Animals

In one aspect, the disclosure relates to mammalian mammary epithelial cells that produce glycosylated antibodies. Methods are provided herein for producing glycosylated antibodies in mammalian mammary epithelial cells.

In some embodiments, the production is accomplished by production in cell culture (i.e., in vitro or ex vivo). Examples of cells of mammary tissue origin include, but are not limited to, mammary gland epithelial cells, mammary epithelial cells, mammary tumor cells (e.g., mammary carcinoma cells). Cells of mammary tissue origin may be primary cells, immortalized cells, or transformed cells.

In some embodiments, the production is accomplished by production in a transgenic animal (i.e., in vivo). In some embodiments, the mammalian mammary gland epithelial cells are in a transgenic animal. In some embodiments, the mammalian mammary gland epithelial cells have been engineered to express recombinant antibodies in the milk of a transgenic animal, such as a mouse or goat. To accomplish this, the expression of the gene(s) encoding the recombinant protein can be, for example, under the control of the goat β-casein regulatory elements. Expression of recombinant proteins, e.g. antibodies, in both mice and goat milk has been established previously (see, e.g., US Patent Application No. 2008-0118501). In some embodiments, the expression is optimized for individual mammary duct epithelial cells that produce milk proteins.

Transgenic animals capable of producing recombinant antibodies can be generated according to methods known in the art (see, e.g., U.S. Pat. No. 5,945,577 and US Patent Application No 2008-0118501). Animals suitable for transgenic expression, include, but are not limited to goat, sheep, bison, camel, cow, pig, rabbit, buffalo, horse, rat, mouse or llama. Suitable animals also include bovine, caprine, ovine and porcine, which relate to various species of cows, goats, sheep and pigs (or swine), respectively. Suitable animals also include ungulates. As used herein, “ungulate” is of or relating to a hoofed typically herbivorous quadruped mammal, including, without limitation, sheep, swine, goats, cattle and horses. Suitable animals also include dairy animals, such as goats and cattle, or mice. In some embodiments, the animal suitable for transgenic expression is a goat.

In some embodiments, transgenic animals are generated by generation of primary cells comprising a construct of interest followed by nuclear transfer of primary cell nuclei into enucleated oocytes. Primary cells comprising a construct of interest are produced by injecting or transfecting primary cells with a single construct comprising the coding sequence of an antibody of interest, e.g., the heavy and light chains of cetuximab, or by co-transfecting or co-injecting primary cells with separate constructs comprising the coding sequences of the heavy and light chains of an antibody, e.g., cetuximab. These cells are then expanded and characterized to assess transgene copy number, transgene structural integrity and chromosomal integration site. Cells with desired transgene copy number, transgene structural integrity and chromosomal integration site are then used for nuclear transfer to produce transgenic animals. As used herein, “nuclear transfer” refers to a method of cloning wherein the nucleus from a donor cell is transplanted into an enucleated oocyte.

Coding sequences for antibodies of interest to be expressed in mammalian mammary epithelial cells can be obtained by screening libraries of genomic material or reverse-translated messenger RNA derived from the animal of choice (such as humans, cattle or mice), from sequence databases such as NCBI, Genbank, or by obtaining the sequences of antibodies using methods known in the art, e.g., peptide mapping. The sequences can be cloned into an appropriate plasmid vector and amplified in a suitable host organism, like E. coli. As used herein, a “vector” may be any of a number of nucleic acids into which a desired sequence may be inserted by restriction and ligation for transport between different genetic environments or for expression in a host cell. Vectors are typically composed of DNA although RNA vectors are also available. Vectors include, but are not limited to, plasmids and phagemids. A cloning vector is one which is able to replicate in a host cell, and which is further characterized by one or more endonuclease restriction sites at which the vector may be cut in a determinable fashion and into which a desired DNA sequence may be ligated such that the new recombinant vector retains its ability to replicate in the host cell. An expression vector is one into which a desired DNA sequence may be inserted by restriction and ligation such that it is operably joined to regulatory sequences and may be expressed as an RNA transcript. Vectors may further contain one or more marker sequences suitable for use in the identification of cells which have or have not been transformed or transfected with the vector. Markers include, for example, genes encoding proteins which increase or decrease either resistance or sensitivity to antibiotics or other compounds, genes which encode enzymes whose activities are detectable by standard assays known in the art (e.g., β-galactosidase or alkaline phosphatase), and genes which visibly affect the phenotype of transformed or transfected cells, hosts, colonies or plaques.

The coding sequence of antibodies or the heavy and light chains of antibodies of interest can be operatively linked to a control sequence which enables the coding sequence to be expressed in the milk of a transgenic non-human mammal. After amplification of the vector, the DNA construct can be digested with restriction enzymes, purified from the remains of the vector and introduced into fertilized embryos to produce transgenic animals. The transgenic animals will have the desired transgenic protein integrated into their genome.

A DNA sequence which is suitable for directing production to the milk of transgenic animals can carry a 5′-promoter region derived from a naturally-derived milk protein. This promoter is consequently under the control of hormonal and tissue-specific factors and is most active in lactating mammary tissue. In some embodiments, the promoter used is a milk-specific promoter. As used herein, a “milk-specific promoter” is a promoter that naturally directs expression of a gene in a cell that secretes a protein into milk (e.g., a mammary epithelial cell) and includes, for example, the casein promoters, e.g., α-casein promoter (e.g., alpha S-1 casein promoter and alpha S2-casein promoter), β-casein promoter (e.g., the goat beta casein gene promoter (DiTullio, BIOTECHNOLOGY 10:74-77, 1992), 65 -casein promoter, κ-casein promoter, whey acidic protein (WAP) promoter (Gorton et al., BIOTECHNOLOGY 5: 1183-1187, 1987), β-lactoglobulin promoter (Clark et al., BIOTECHNOLOGY 7: 487-492, 1989) and α-lactalbumin promoter (Soulier et al., FEBS LETTS. 297:13, 1992). Also included in this definition are promoters that are specifically activated in mammary tissue, such as, for example, the long terminal repeat (LTR) promoter of the mouse mammary tumor virus (MMTV). In some embodiments the promoter is a caprine beta casein promoter.

The promoter can be operably linked to a DNA sequence directing the production of a protein leader sequence which directs the secretion of the transgenic protein across the mammary epithelium into the milk. As used herein, a coding sequence and regulatory sequences (e.g., a promoter) are said to be “operably joined” or “operably linked” when they are linked in such a way as to place the expression or transcription of the coding sequence under the influence or control of the regulatory sequences. As used herein, a “leader sequence” or “signal sequence” is a nucleic acid sequence that encodes a protein secretory signal, and, when operably linked to a downstream nucleic acid molecule encoding a transgenic protein directs secretion. The leader sequence may be the native human leader sequence, an artificially-derived leader, or may be obtained from the same gene as the promoter used to direct transcription of the transgene coding sequence, or from another protein that is normally secreted from a cell, such as a mammalian mammary epithelial cell.

In some embodiments a 3′-sequence, which can be derived from a naturally secreted milk protein, can be added to improve stability of mRNA. In some embodiments, to produce primary cell lines containing a construct (e.g., encoding an cetuximab antibody) for use in producing transgenic goats by nuclear transfer, the heavy and light chain constructs can be transfected into primary goat skin epithelial cells, which are expanded and fully characterized to assess transgene copy number, transgene structural integrity and chromosomal integration site. As used herein, “nuclear transfer” refers to a method of cloning wherein the nucleus from a donor cell is transplanted into an enucleated oocyte.

Cloning will result in a multiplicity of transgenic animals—each capable of producing an antibody or other gene construct of interest. The production methods include the use of the cloned animals and the offspring of those animals. Cloning also encompasses the nuclear transfer of fetuses, nuclear transfer, tissue and organ transplantation and the creation of chimeric offspring. One step of the cloning process comprises transferring the genome of a cell, e.g. a primary cell that contains the transgene of interest into an enucleated oocyte. As used herein, “transgene” refers to any piece of a nucleic acid molecule that is inserted by artifice into a cell, or an ancestor thereof, and becomes part of the genome of an animal which develops from that cell. Such a transgene may include a gene which is partly or entirely exogenous (i.e., foreign) to the transgenic animal, or may represent a gene having identity to an endogenous gene of the animal. Suitable mammalian sources for oocytes include goats, sheep, cows, pigs, rabbits, guinea pigs, mice, hamsters, rats, non-human primates, etc. Preferably, oocytes are obtained from ungulates, and most preferably goats or cattle. Methods for isolation of oocytes are well known in the art. Essentially, the process comprises isolating oocytes from the ovaries or reproductive tract of a mammal, e.g., a goat. A readily available source of ungulate oocytes is from hormonally-induced female animals. For the successful use of techniques such as genetic engineering, nuclear transfer and cloning, oocytes may preferably be matured in vivo before these cells may be used as recipient cells for nuclear transfer, and before they were fertilized by the sperm cell to develop into an embryo. Metaphase II stage oocytes, which have been matured in vivo, have been successfully used in nuclear transfer techniques. Essentially, mature metaphase II oocytes are collected surgically from either non-super ovulated or super ovulated animals several hours past the onset of estrus or past the injection of human chorionic gonadotropin (hCG) or similar hormone.

Thus, in one aspect the disclosure provides mammary gland epithelial cells that produce the antibodies or compositions of the disclosure. In some embodiments, the antibody comprises a nucleic acid comprising SEQ ID NO:3 and a nucleic acid comprising SEQ ID NO:4. In some embodiments, the nucleic acid comprising SEQ ID NO:3 and the nucleic acid comprising SEQ ID NO:4 are connected. “Connected” is used herein to mean the nucleic acids are physically linked, e.g., within the same vector or within approximately the same genomic location. In some embodiments, the mammary epithelial cells above are in a transgenic non-human mammal. In some embodiments, the transgenic non-human mammal is a goat.

In another aspect the disclosure provides a method for the production of a transgenic antibody, and variants and fragments thereof, the process comprising expressing in the milk of a transgenic non-human mammal a transgenic antibody encoded by a nucleic acid construct. In some embodiments, the method for producing the antibodies of the disclosure comprises:

(a) transfecting non-human mammalian cells with a transgene DNA construct encoding a cetuximab antibody;

(b) selecting cells in which said cetuximab transgene DNA construct has been inserted into the genome of the cells; and

(c) performing a first nuclear transfer procedure to generate a non-human transgenic mammal heterozygous for the cetuximab antibody and that can express it in its milk.

In some embodiments, the transgene DNA construct comprises SEQ ID NO:3 and/or SEQ ID NO:4. In some embodiments, the non-human transgenic mammal is a goat. In another aspect, the disclosure provides a method of:

(a) providing a non-human transgenic mammal engineered to express a cetuximab antibody,

(b) expressing the cetuximab antibody in the milk of the non-human transgenic mammal; and

(c) isolating the cetuximab antibody expressed in the milk.

In some embodiments, the cetuximab antibody comprises a heavy chain comprising SEQ ID NO:1 and a light chain comprising SEQ ID NO:2. One of the tools used to predict the quantity and quality of the recombinant protein expressed in the mammary gland is through the induction of lactation (Ebert K M, 1994). Induced lactation allows for the expression and analysis of protein from the early stage of transgenic production rather than from the first natural lactation resulting from pregnancy, which is at least a year later. Induction of lactation can be done either hormonally or manually.

In some embodiments, the compositions of glycosylated antibodies provided herein further comprise milk. In some embodiments, the methods provided herein include a step of isolating a population of antibodies from the milk of a transgenic animal. Methods for isolating antibodies from the milk of transgenic animal are known in the art and are described for instance in Pollock et al., Journal of Immunological Methods, Volume 231, Issues 1-2, 10 Dec. 1999, Pages 147-157. In some embodiments, the methods provided herein include a step of purifying glycosylated antibodies with a desired amount of gal-alpha-1,3-gal.

### Pharmaceutical Compositions, Dosage, and Administration

In one aspect, the disclosure provides pharmaceutical compositions which comprise an amount of an antibody of interest or a pharmaceutically acceptable salt of said antibody and a pharmaceutically acceptable vehicle, diluent or carrier.

In another aspect the disclosure provides a method of treating a subject, comprising administering to a subject a composition provided in an amount effective to treat a disease the subject has or is at risk of having is provided. In one embodiment the subject is a human. In another embodiment the subject is a non-human animal, e.g. a dog, cat, horse, cow, pig, sheep, goat or primate. In some embodiments, the disease is cancer. In some embodiments, the cancer comprises cancer cells expressing EGFR. In some embodiments, the cancer comprises cells with a wild-type KRAS gene. In some embodiments, the cancer is head and neck cancer. In some embodiments, the head and neck cancer is squamous cell carcinoma. In some embodiments, the cancer is colorectal cancer. In some embodiments, the colorectal cancer is metastatic colorectal cancer.

Some methods contemplate combination therapy with known cancer medicaments or therapies, for example, chemotherapeutic agents, immunotherapeutic agents, cancer vaccines, hormone therapy, biological response modifiers, surgical procedures, or radiation therapy. It is to be understood that the administering of the antibodies or compositions of the disclosure and the administering of another medicament or therapy can occur simultaneously or non-simultaneously. In one embodiment, the combination therapy comprises administering the antibodies and compositions provided herein in combination with radiation therapy.

In another embodiment the subject is further administered an additional cancer medicament. In one embodiment the additional cancer medicament is a chemotherapeutic agent. In some embodiments, the chemotherapeutic agent is fluorouracil (also referred to as 5-fluorouracil or 5-FU). In some embodiments, the chemotherapeutic agent is irinotecan. In some embodiments, combination therapy comprises:

(a) an antibody or a pharmaceutical composition of the disclosure (e.g., cetuximab);

(b) leucovorin calcium;

(c) fluorouracil; and

(d) irinotecan hydrochloride.

The combination of (b), (c), and (d) is referred to as FOLFIRI in the art. Thus, in some embodiments, methods of treatment comprise administering an antibody or a pharmaceutical composition of the disclosure and FOLFIRI.

According to embodiments that involve administering to a subject in need of treatment a therapeutically effective amount of the antibodies as provided herein, “therapeutically effective” or “an amount effective to treat” denotes the amount of antibody or of a composition needed to inhibit or reverse a disease condition (e.g., reduce or inhibit cancer growth). Determining a therapeutically effective amount specifically depends on such factors as toxicity and efficacy of the medicament. These factors will differ depending on other factors such as potency, relative bioavailability, patient body weight, severity of adverse side-effects and preferred mode of administration. Toxicity may be determined using methods well known in the art. Efficacy may be determined utilizing the same guidance. Efficacy, for example, can be measured by a decrease in mass of a target tissue, e.g. a tumor. A pharmaceutically effective amount, therefore, is an amount that is deemed by the clinician to be toxicologically tolerable, yet efficacious.

Dosage may be adjusted appropriately to achieve desired drug levels, local or systemic, depending upon the mode of administration. In the event that the response in a subject is insufficient at such doses, even higher doses (or effective higher doses by a different, more localized delivery route) may be employed to the extent that patient tolerance permits. Multiple doses per day are contemplated to achieve appropriate systemic levels of antibodies. Appropriate systemic levels can be determined by, for example, measurement of the patient's peak or sustained plasma level of the drug. “Dose” and “dosage” are used interchangeably herein.

In some embodiments, the amount of antibody or pharmaceutical composition administered to a subject is 50 to 500 mg/m², 100 to 400 mg/m², or 200 to 300 mg/m² per week. In one embodiment the amount of antibody or pharmaceutical composition administered to a subject is 250 mg/m² per week. In some embodiments, an initial dose of 400 mg/m² is administered to a subject the first week, followed by administration of 250 mg/m² to the subject in subsequent weeks. In some embodiments the administration rate is less than 10mg/min In some embodiments, administration of the antibody or pharmaceutical composition to a subject occurs at least one hour prior to treatment with another therapeutic agent, e.g., 5-FU or FOLFIRI. In some embodiments, a pre-treatment is administered prior to administration of the antibody or pharmaceutical composition. In one embodiment, the pre-treatment is administration of an H₁ antagonist. In one embodiment, the H₁ antagonist if diphenhydramine.

In some embodiments the compositions provided are employed for in vivo applications. Depending on the intended mode of administration in vivo the compositions used may be in the dosage form of solid, semi-solid or liquid such as, e.g., tablets, pills, powders, capsules, gels, ointments, liquids, suspensions, or the like. Preferably the compositions are administered in unit dosage forms suitable for single administration of precise dosage amounts. The compositions may also include, depending on the formulation desired, pharmaceutically acceptable carriers or diluents, which are defined as aqueous-based vehicles commonly used to formulate pharmaceutical compositions for animal or human administration. The diluent is selected so as not to affect the biological activity of the human recombinant protein of interest. Examples of such diluents are distilled water, physiological saline, Ringer's solution, dextrose solution, and Hank's solution. The same diluents may be used to reconstitute a lyophilized recombinant protein of interest. In addition, the pharmaceutical composition may also include other medicinal agents, pharmaceutical agents, carriers, adjuvants, nontoxic, non-therapeutic, non-immunogenic stabilizers, etc. Effective amounts of such diluent or carrier are amounts which are effective to obtain a pharmaceutically acceptable formulation in terms of solubility of components, biological activity, etc. In some embodiments the compositions provided herein are sterile.

Administration during in vivo treatment may be by any number of routes, including oral, parenteral, intramuscular, intranasal, sublingual, intratracheal, inhalation, ocular, vaginal, and rectal. Intracapsular, intravenous, and intraperitoneal routes of administration may also be employed. The skilled artisan recognizes that the route of administration varies depending on the disorder to be treated. For example, the compositions or antibodies herein may be administered to a subject via oral, parenteral or topical administration and otherwise systemic forms for anti-cancer treatment, e.g. head and neck cancer or colorectal cancer. In one embodiment, the compositions or antibodies herein are administered by intravenous infusion.

The compounds, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions. Alternatively, the active compounds may be in powder form for constitution with a suitable vehicle, e.g., sterile pyrogen-free water, before use.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate. The component or components may be chemically modified so that oral delivery of the antibodies is efficacious. Generally, the chemical modification contemplated is the attachment of at least one molecule to the antibodies, where said molecule permits (a) inhibition of proteolysis; and (b) uptake into the blood stream from the stomach or intestine. Also desired is the increase in overall stability of the antibodies and increase in circulation time in the body. Examples of such molecules include: polyethylene glycol, copolymers of ethylene glycol and propylene glycol, carboxymethyl cellulose, dextran, polyvinyl alcohol, polyvinyl pyrrolidone and polyproline. Abuchowski and Davis, 1981, “Soluble Polymer-Enzyme Adducts” In: Enzymes as Drugs, Hocenberg and Roberts, eds., Wiley-Interscience, New York, N.Y., pp. 367-383; Newmark, et al., 1982, J. Appl. Biochem. 4:185-189. Other polymers that could be used are poly-1,3-dioxolane and poly-1,3,6-tioxocane. Preferred for pharmaceutical usage, as indicated above, are polyethylene glycol molecules. For oral compositions, the location of release may be the stomach, the small intestine (the duodenum, the jejunum, or the ileum), or the large intestine. One skilled in the art has available formulations which will not dissolve in the stomach, yet will release the material in the duodenum or elsewhere in the intestine. Preferably, the release will avoid the deleterious effects of the stomach environment, either by protection of the antibody or by release of the biologically active material beyond the stomach environment, such as in the intestine.

For buccal administration, the compositions may take the form of tablets or lozenges formulated in conventional manner.

For administration by inhalation, the compounds for use according to the present disclosure may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

Also contemplated herein is pulmonary delivery. The compositions can be delivered to the lungs of a mammal while inhaling and traverses across the lung epithelial lining to the blood stream. Contemplated for use in the practice of this disclosure are a wide range of mechanical devices designed for pulmonary delivery of therapeutic products, including but not limited to nebulizers, metered dose inhalers, and powder inhalers, all of which are familiar to those skilled in the art.

Nasal delivery of a pharmaceutical composition of the present disclosure is also contemplated. Nasal delivery allows the passage of a pharmaceutical composition of the present disclosure to the blood stream directly after administering the therapeutic product to the nose, without the necessity for deposition of the product in the lung. Formulations for nasal delivery include those with dextran or cyclodextran.

The compounds may also be formulated in rectal or vaginal compositions such as suppositories or retention enemas, e.g., containing conventional suppository bases such as cocoa butter or other glycerides.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer, Science 249:1527-1533, 1990, which is incorporated herein by reference.

The antibodies and optionally other therapeutics may be administered per se (neat) or in the form of a pharmaceutically acceptable salt. When used in medicine the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

The pharmaceutical compositions of the disclosure contain an effective amount of the antibodies and optionally therapeutic agents included in a pharmaceutically-acceptable carrier. The term pharmaceutically-acceptable carrier means one or more compatible solid or liquid filler, diluents or encapsulating substances which are suitable for administration to a human or other vertebrate animal. The term carrier denotes an organic or inorganic ingredient, natural or synthetic, with which the active ingredient is combined to facilitate the application. The components of the pharmaceutical compositions also are capable of being commingled with the compounds of the present disclosure, and with each other, in a manner such that there is no interaction which would substantially impair the desired pharmaceutical efficiency.

The therapeutic agent(s), including specifically but not limited to the antibodies, may be provided in particles. Particles, as used herein, include nano or microparticles (or in some instances larger) which can consist in whole or in part of the antibody or other therapeutic agents administered with the antibody. The particle may include, in addition to the therapeutic agent(s), any of those materials routinely used in the art of pharmacy and medicine, including, but not limited to, erodible, nonerodible, biodegradable, or nonbiodegradable material or combinations thereof. The particles may be microcapsules which contain the antibody in a solution or in a semi-solid state. The particles may be of virtually any shape.

### Methods of Production

One aspect of the disclosure provides a method for the production of a glycosylated antibody or population of glycosylated antibodies, the process comprising expressing in the milk of a transgenic non-human mammal a glycosylated antibody encoded by a nucleic acid construct. In one embodiment the mammalian mammary epithelial cells are of a non-human mammal engineered to express the antibody in its milk. In yet another embodiment the mammalian mammary epithelial cells are mammalian mammary epithelial cells in culture.

In another embodiment the method comprises:

(a) providing a non-human transgenic mammal engineered to express an antibody,

(b) expressing the antibody in the milk of the non-human transgenic mammal;

(c) isolating the antibodies expressed in the milk; and

(d) detecting the presence of gal-alpha-1,3-gal moieties on the isolated antibodies.

In yet another embodiment, the method comprises producing a population of glycosylated antibodies in mammary gland epithelial cells such that the population of glycosylated antibodies produced comprises fewer galactose-alpha-1,3-galactose moieties than the population of glycosylated antibodies produced in non-mammary cell culture, wherein the glycosylated antibodies comprise a heavy chain comprising SEQ ID NO:1 and a light chain comprising SEQ ID NO:2. In some embodiments, this method is performed in vitro. In other embodiments, this method is performed in vivo, e.g. in the mammary gland of a transgenic goat.

In some embodiments the methods above further comprise steps for inducing lactation. In still other embodiments the methods further comprise additional isolation and/or purification steps. In yet other embodiments the methods further comprise steps for comparing the glycosylation pattern of the antibodies obtained with antibodies produced in cell culture, e.g. non-mammary cell culture. In further embodiments, the methods further comprise steps for comparing the glycosylation pattern of the antibodies obtained to antibodies produced by non-mammary epithelial cells. Such cells can be cells of a cell culture. In some embodiments, the glycosylation pattern is the gal-alpha-1,3-gal pattern, e.g. the amount of gal-alpha-1,3-gal moieties present on an antibody or population of antibodies. In some embodiments, the method further comprises comparing the number of galactose-alpha-1,3-galactose moieties present in the population of glycosylated antibodies to the number of galactose-alpha-1,3-galactose moieties present in a population of glycosylated antibodies produced in cell culture, e.g. non-mammary cell culture. Experimental techniques for assessing the glycosylation pattern of the antibodies can be any of those known to those of ordinary skill in the art or as provided herein, such as below in the Examples. Such methods include, e.g., liquid chromatography mass spectrometry, tandem mass spectrometry, and Western blot analysis.

The antibodies can be obtained, in some embodiments, by collecting the antibodies from the milk of a transgenic animal produced as provided herein or from an offspring of said transgenic animal. In some embodiments the antibodies produced by the transgenic mammal is produced at a level of at least 1 gram per liter of milk produced.

Unless otherwise defined herein, scientific and technical terms used in connection with the present disclosure shall have the meanings that are commonly understood by those of ordinary skill in the art. Further, unless otherwise required by context, singular terms shall include pluralities and plural terms shall include the singular. The methods and techniques of the present disclosure are generally performed according to conventional methods well-known in the art. Generally, nomenclatures used in connection with, and techniques of biochemistry, enzymology, molecular and cellular biology, microbiology, genetics and protein and nucleic acid chemistry and hybridization described herein are those well-known and commonly used in the art. The methods and techniques of the present disclosure are generally performed according to conventional methods well known in the art and as described in various general and more specific references that are cited and discussed throughout the present specification unless otherwise indicated.

The present disclosure is further illustrated by the following Examples, which in no way should be construed as further limiting. The entire contents of all of the references (including literature references, issued patents, published patent applications, and co pending patent applications) cited throughout this application are hereby expressly incorporated by reference.

### EXAMPLES

### Methods

TABLE 1
SEQ ID NO list

| SEQ ID | |
|---|---|
| NO | Description |
| 1 | Heavy chain amino acid sequence of cetuximab |
| 2 | Light chain amino acid sequence of cetuximab |
| 3 | Heavy chain nucleotide sequence of cetuximab |
| 4 | Light chain nucleotide sequence of cetuximab |
| 5 | Bc2553 |
| 6 | Bc2554 |
| 7 | Bc2584 |

### Gene Encoding Cetuximab

The amino acid sequence of cetuximab/Erbitux® was published in Charlotte Magdelaine-Beuzelin, et al., Critical Reviews in Oncology/Hematology 64 (2007) 210-225 and used as a reference for confirmation of the sequence of commercially-produced Erbitux®.

### Peptide Map

The commercially produced cetuximab, Erbitux®, was analyzed through generation of the peptide maps of the heavy and light chain and the published amino acid sequence was confirmed. The peptide maps were generated though trypsin and Asp-N digestion, followed by HPLC and mass spec analysis. The amino acid sequences of commercially produced cetuximab were then used to design DNA expression sequences for expression of cetuximab in the milk of transgenic animals.

### Beta Casein Expression Constructs

The DNA sequences of the cetuximab heavy and light chains were optimized for expression in a bovine system (SEQ ID NOs: 3 (heavy chain) and 4 (light chain)). All constructs described herein contain the optimized sequences.

The constructs used herein include the coding sequence of cetuximab, a Kozak consensus sequence as well as flanking XhoI sites to facilitate cloning into beta casein expression vectors. The expression vectors used for this project are Bc450, Bc451, Bc800, and Bc350, all of which were in the supercos background which confers both Amp^{R} and Kan^{R}.

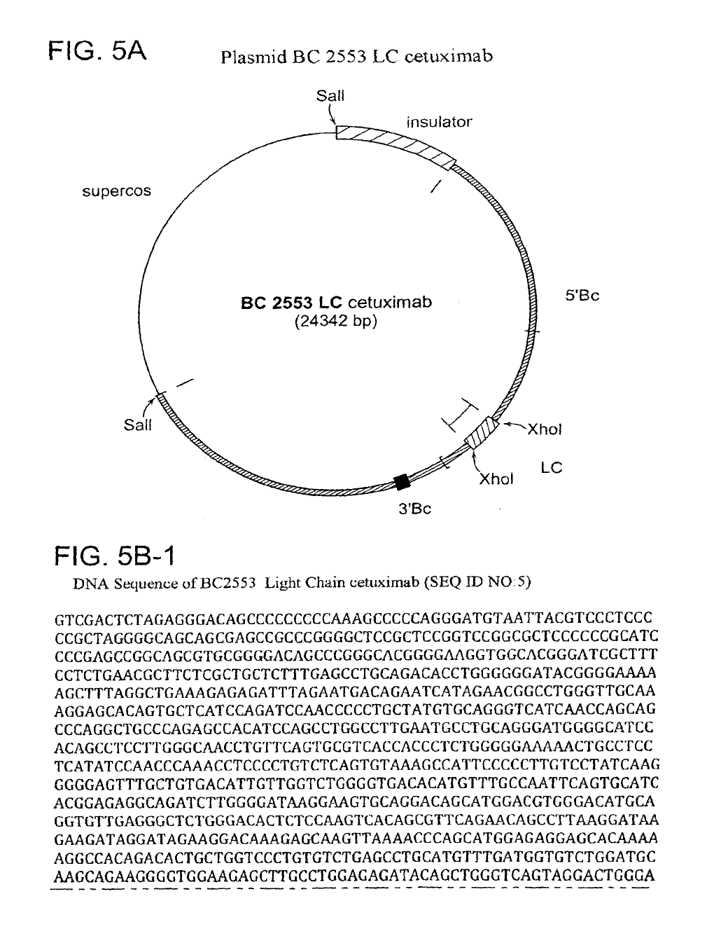

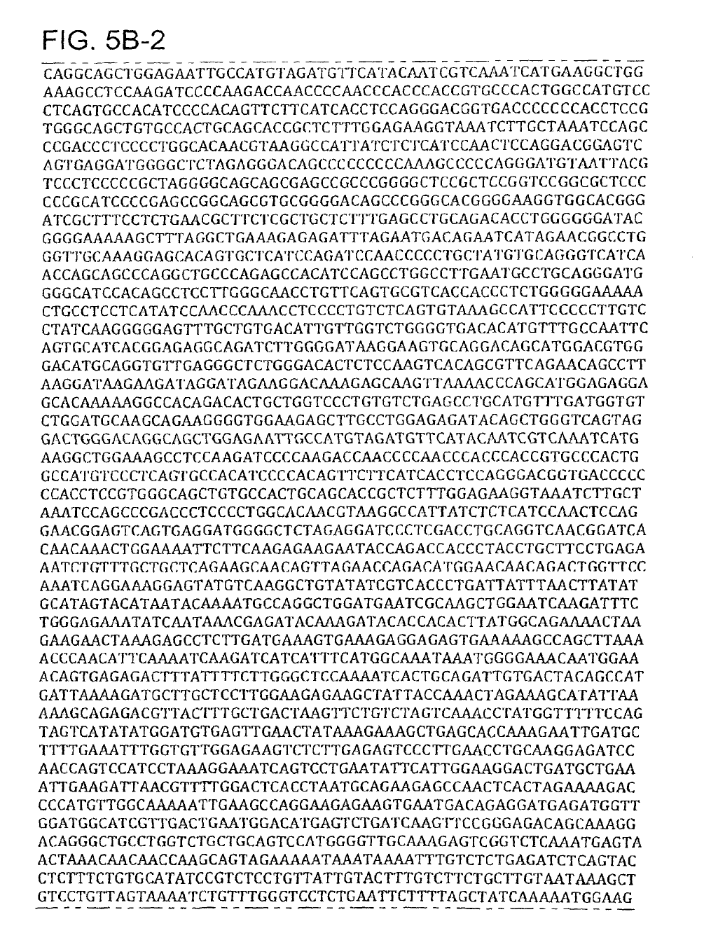

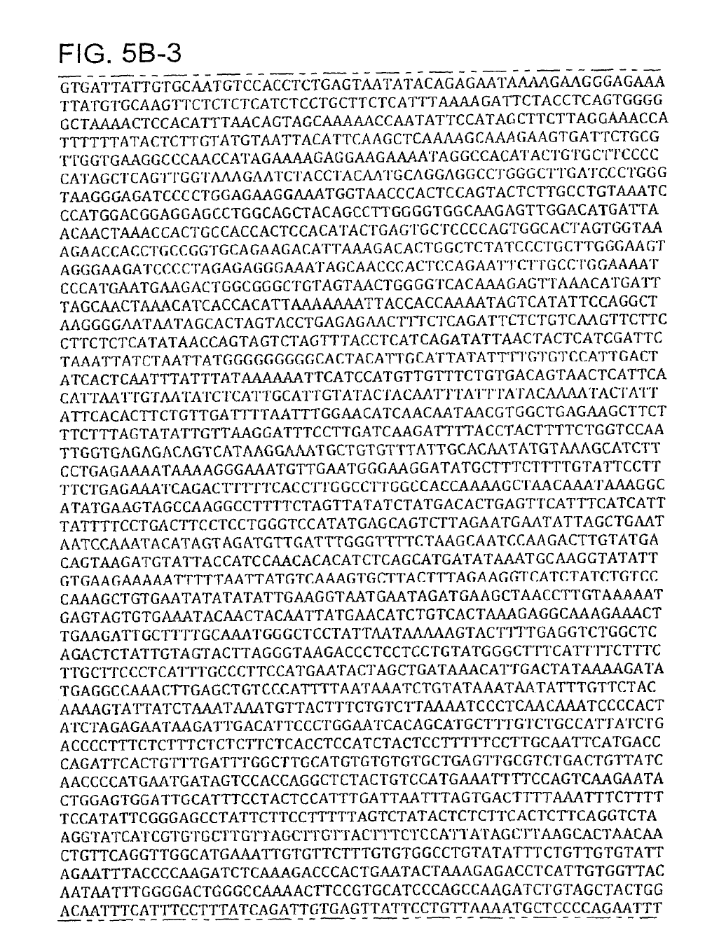

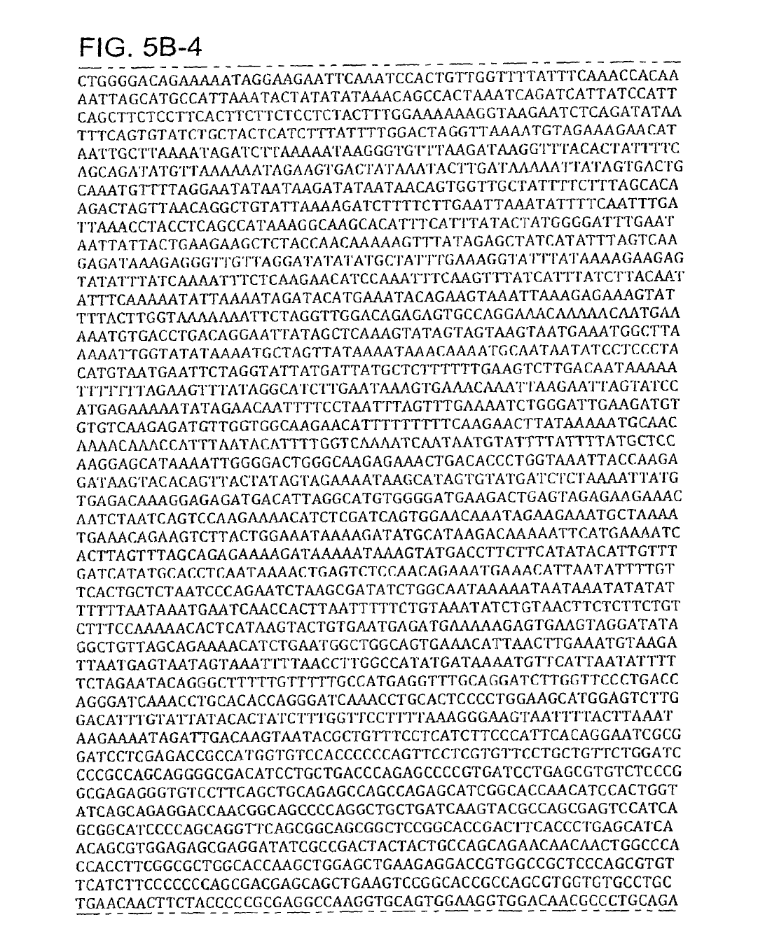

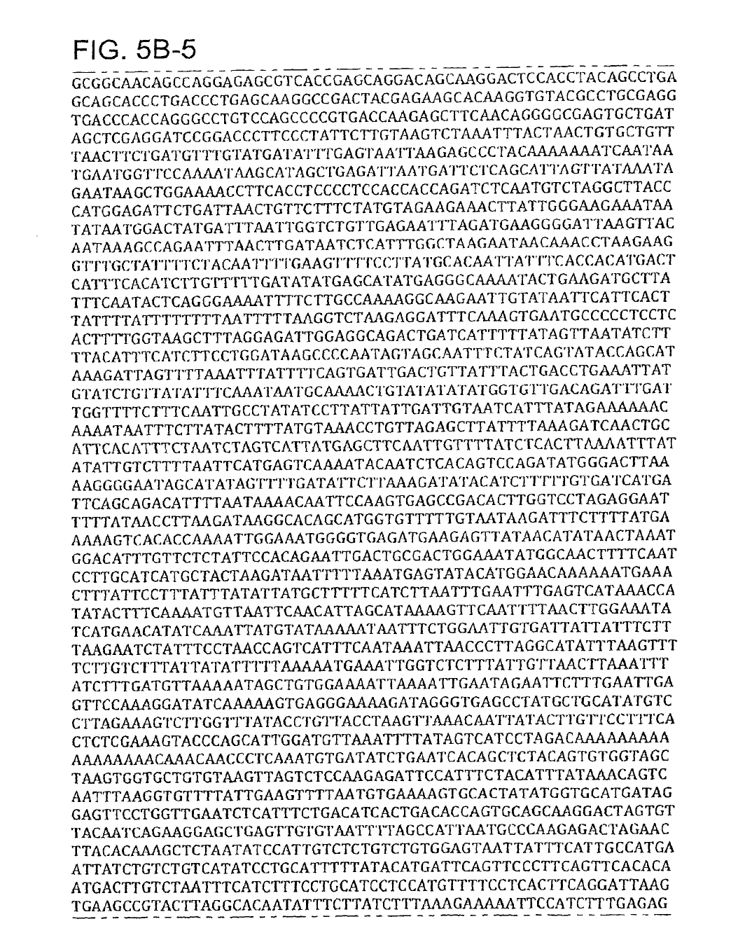

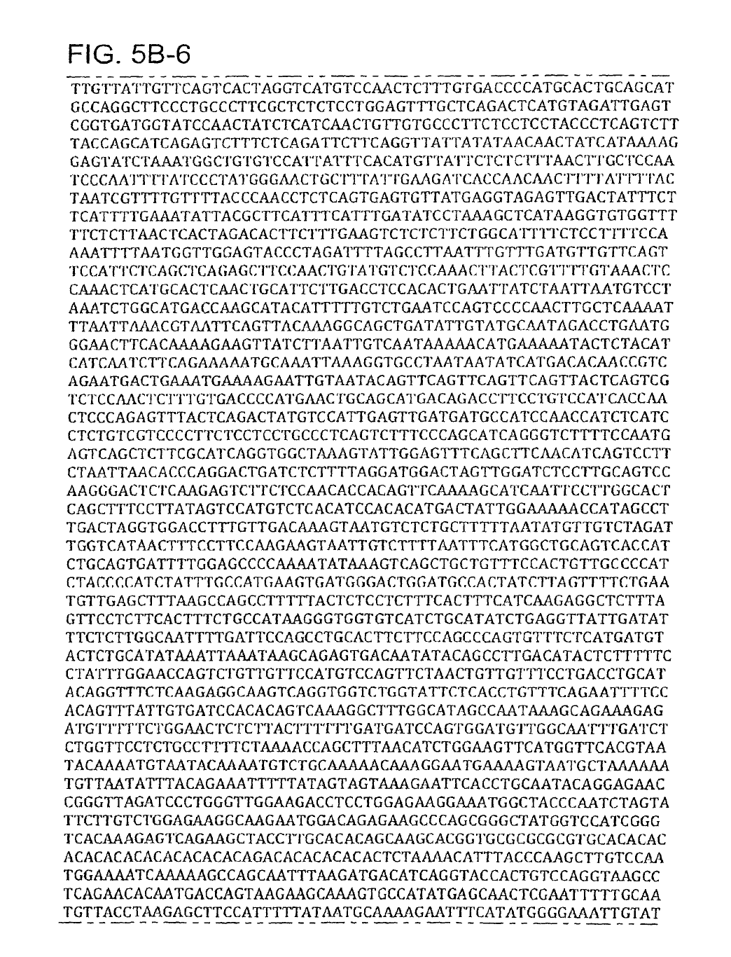

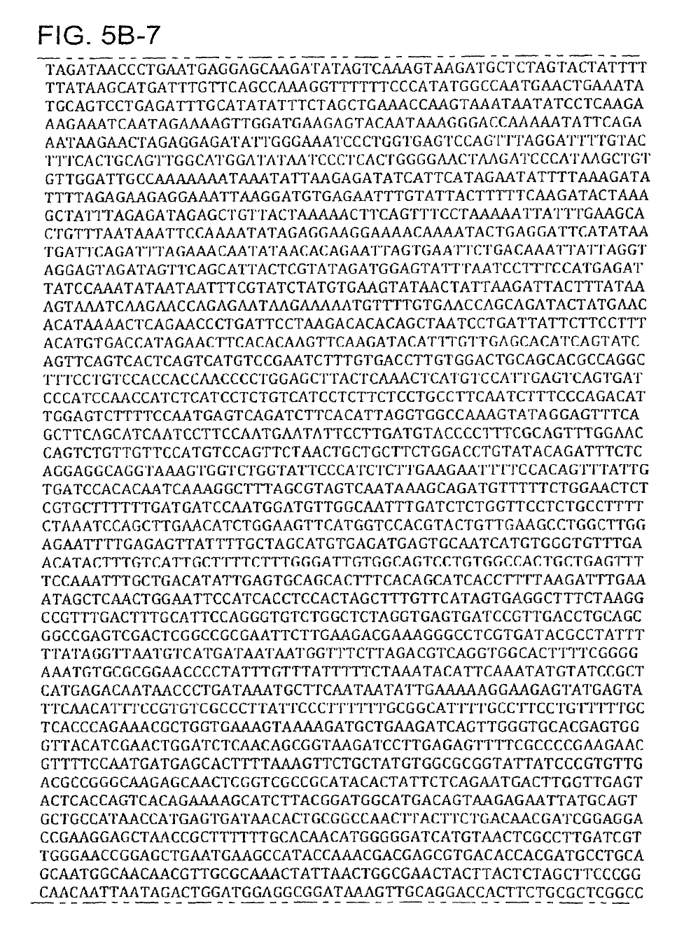

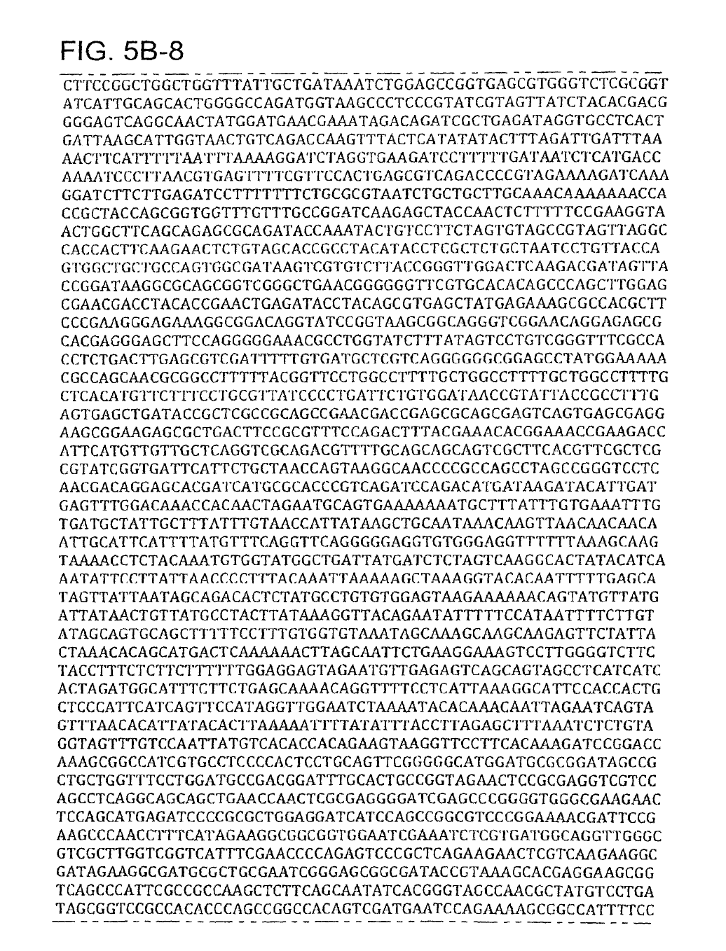

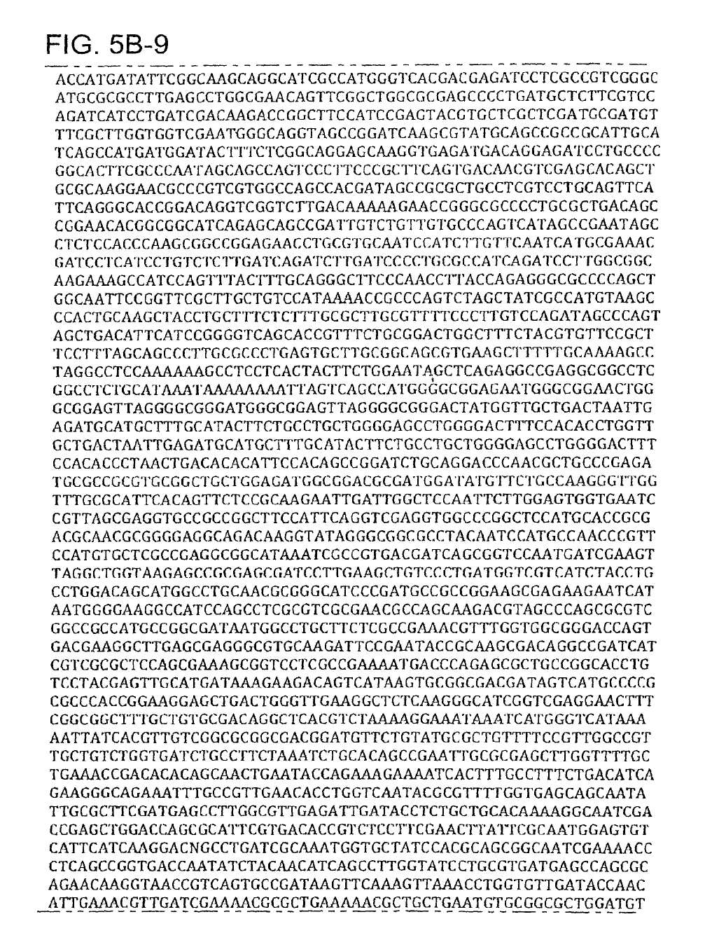

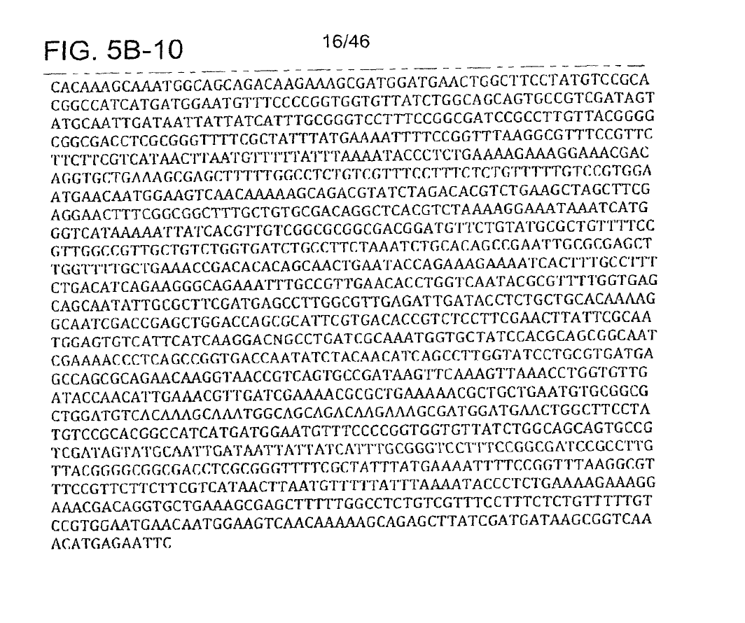

Plasmid Bc2553 (FIG. 5) contains the cetuximab light chain nucleotide sequence (SEQ ID NO:4) in expression vector Bc450, which includes a chicken beta globin insulator sequence, 6.2 kb of 5′ beta casein sequence, an XhoI cloning site, and 7.1 kb of 3′ beta casein flanking sequence, in a supercos backbone. The supercos vector can be removed using flanking SalI sites.

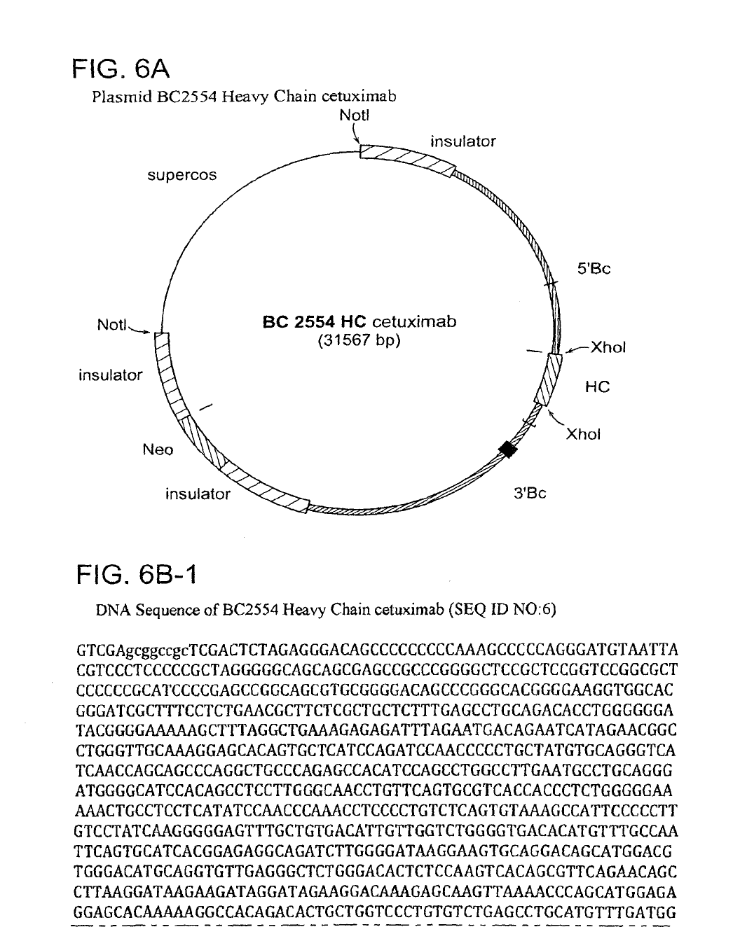

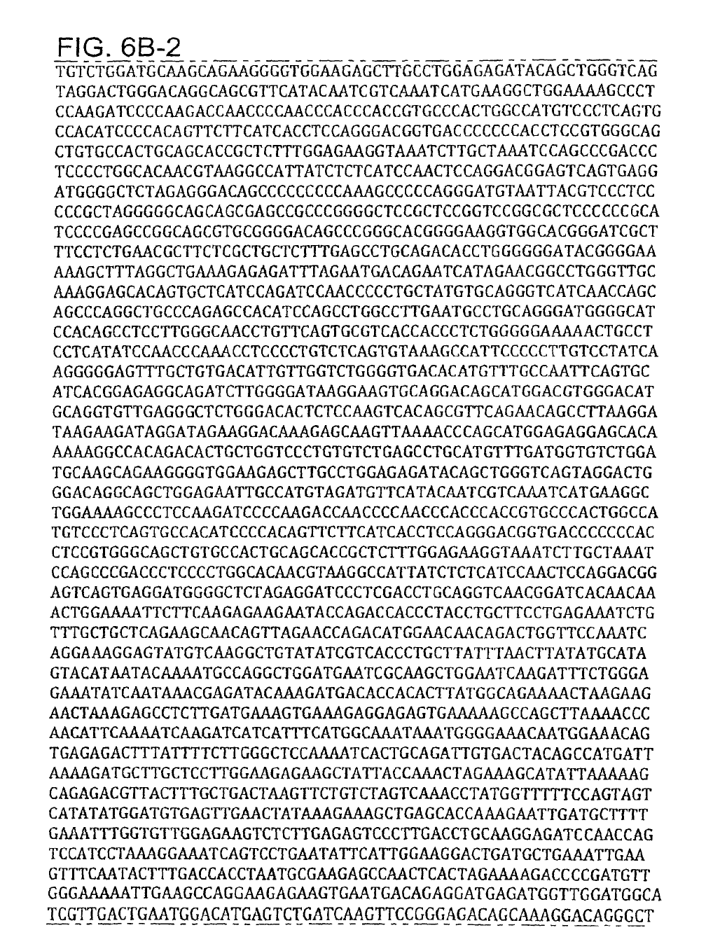

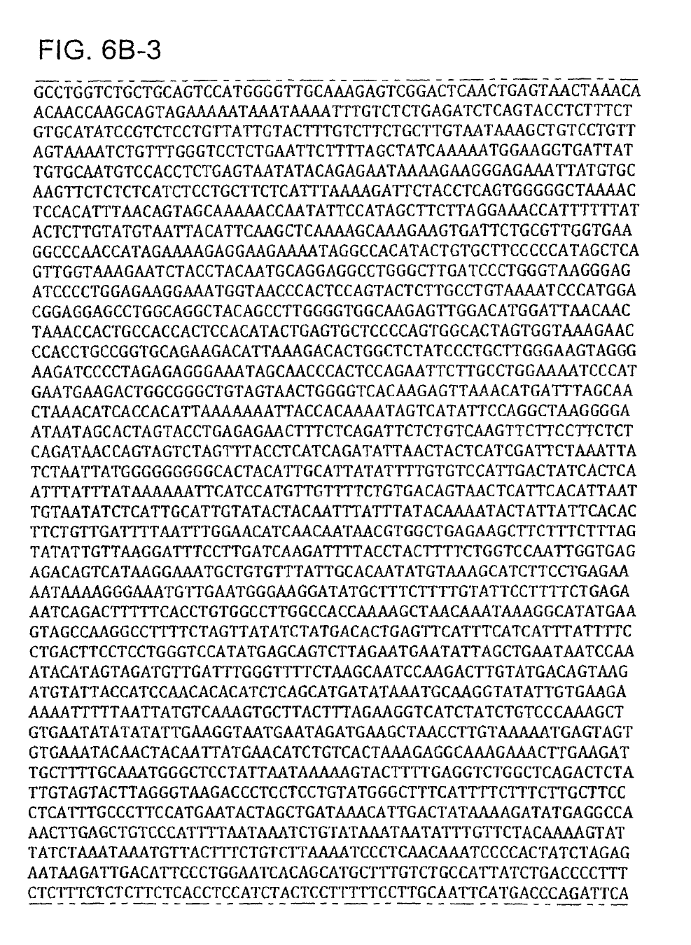

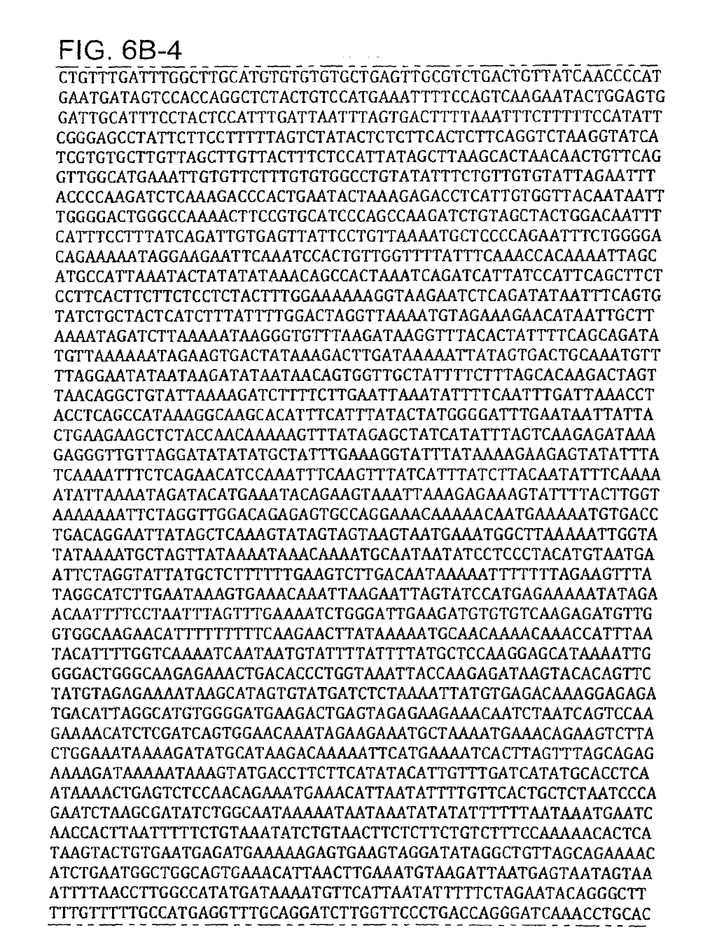

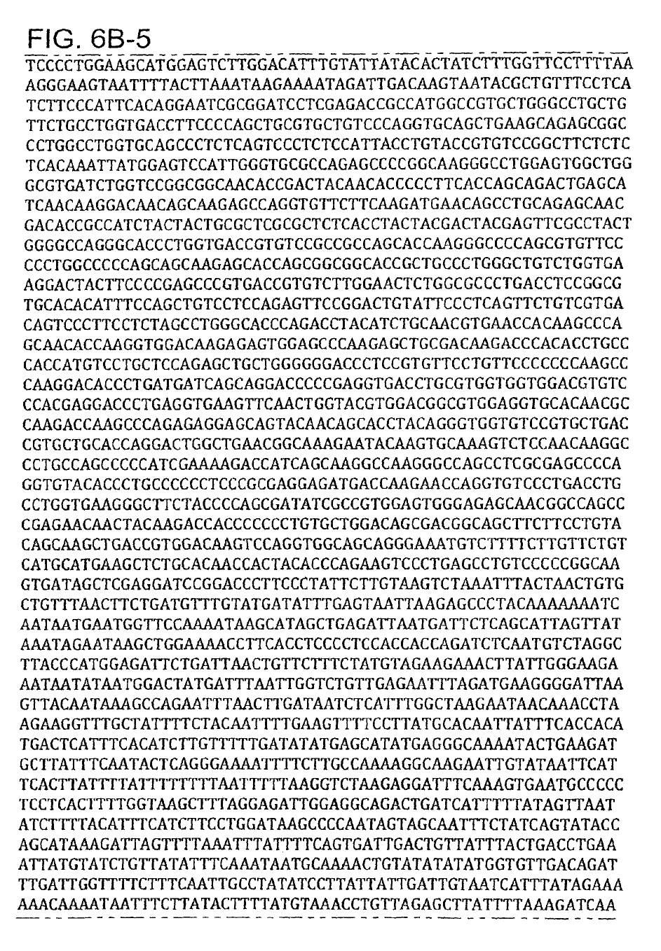

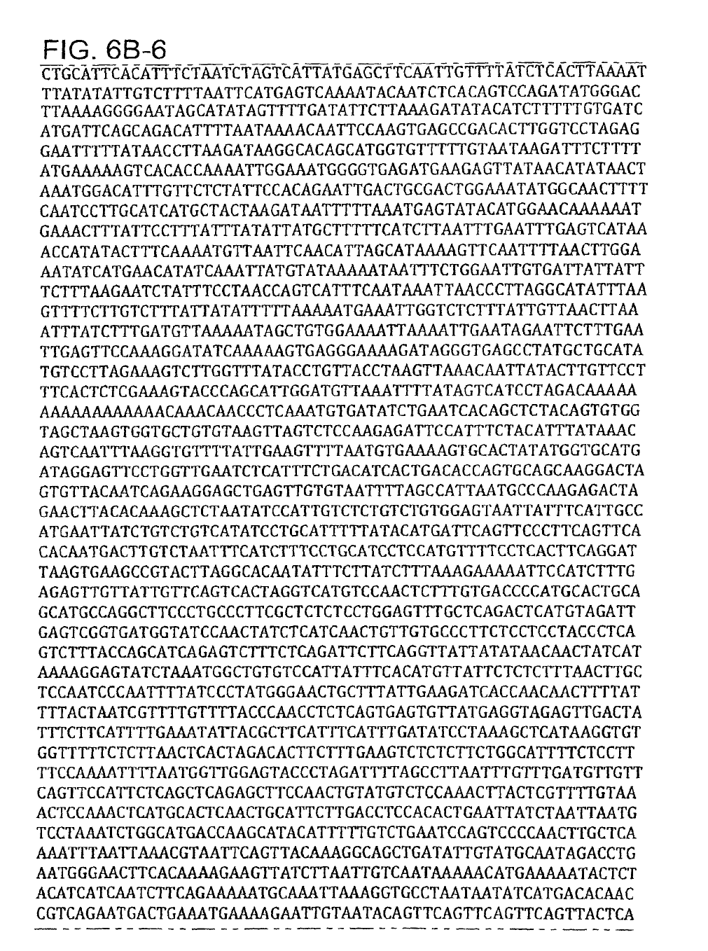

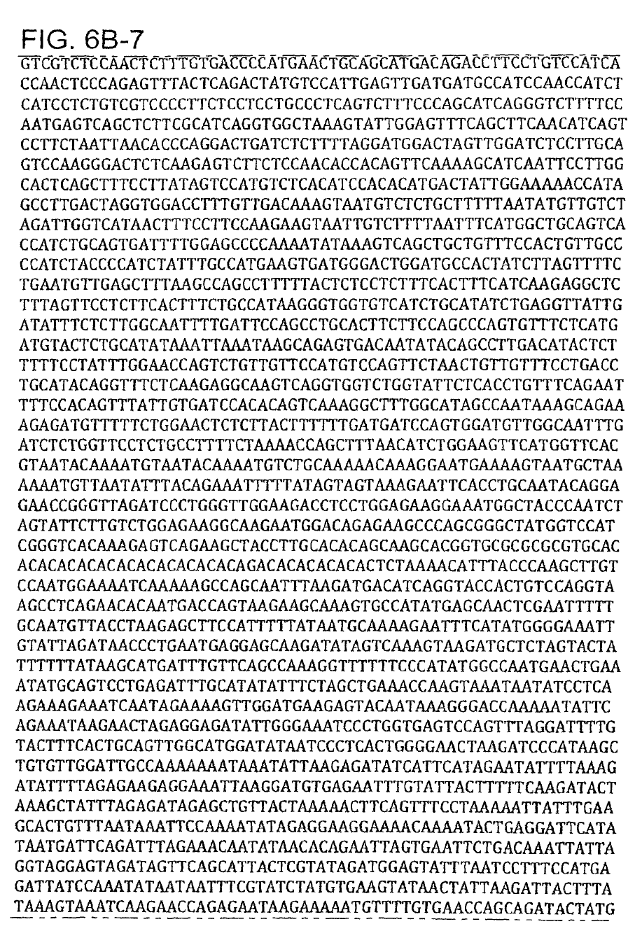

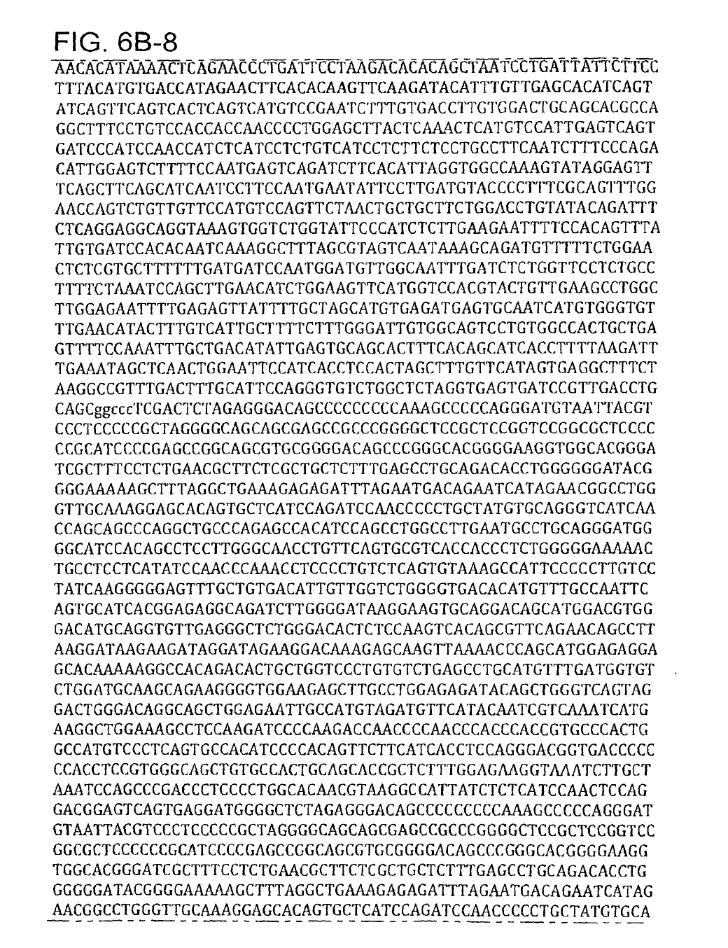

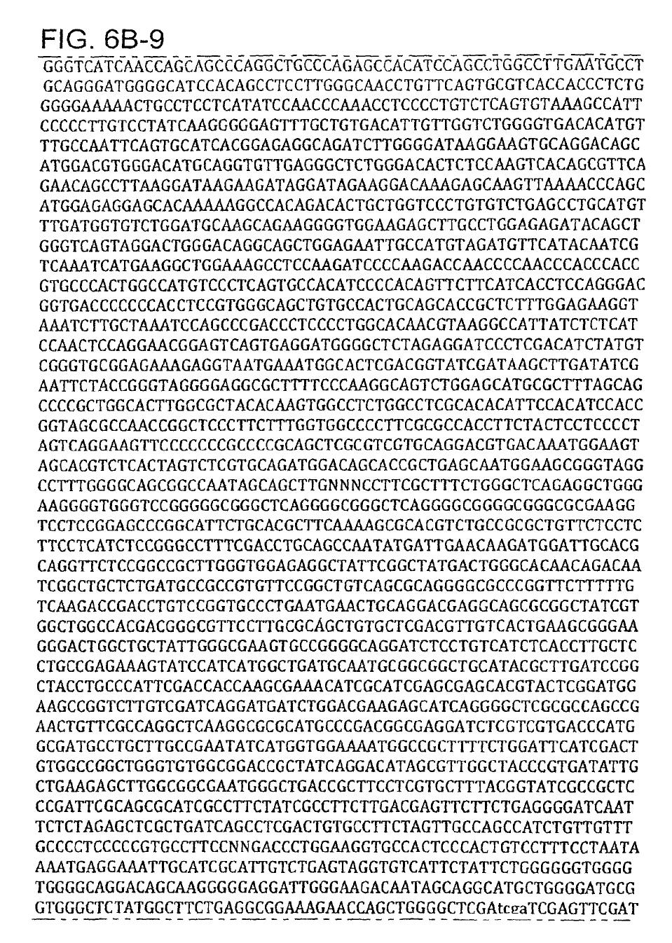

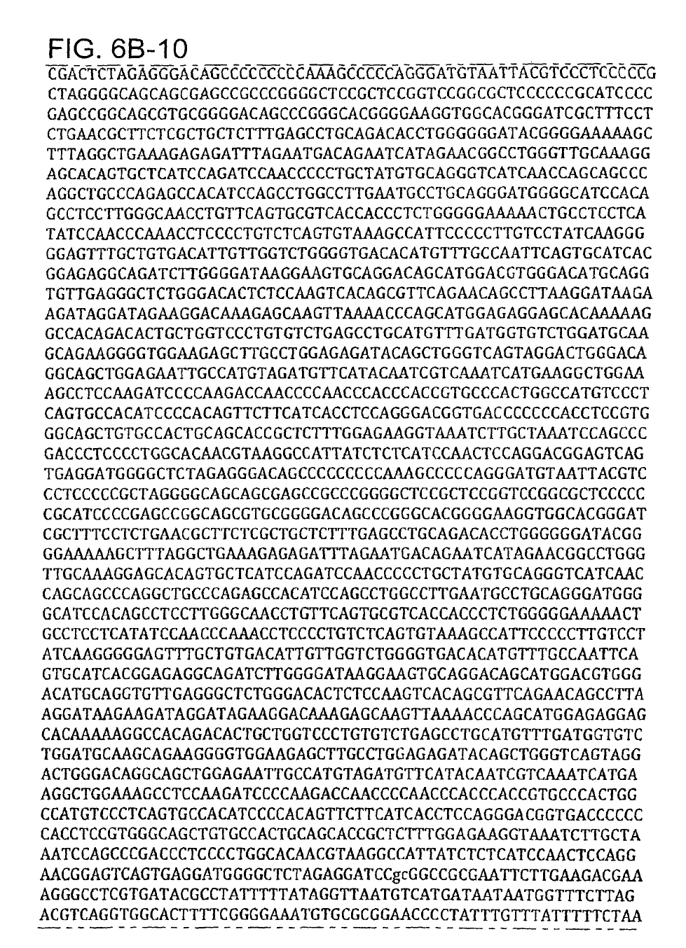

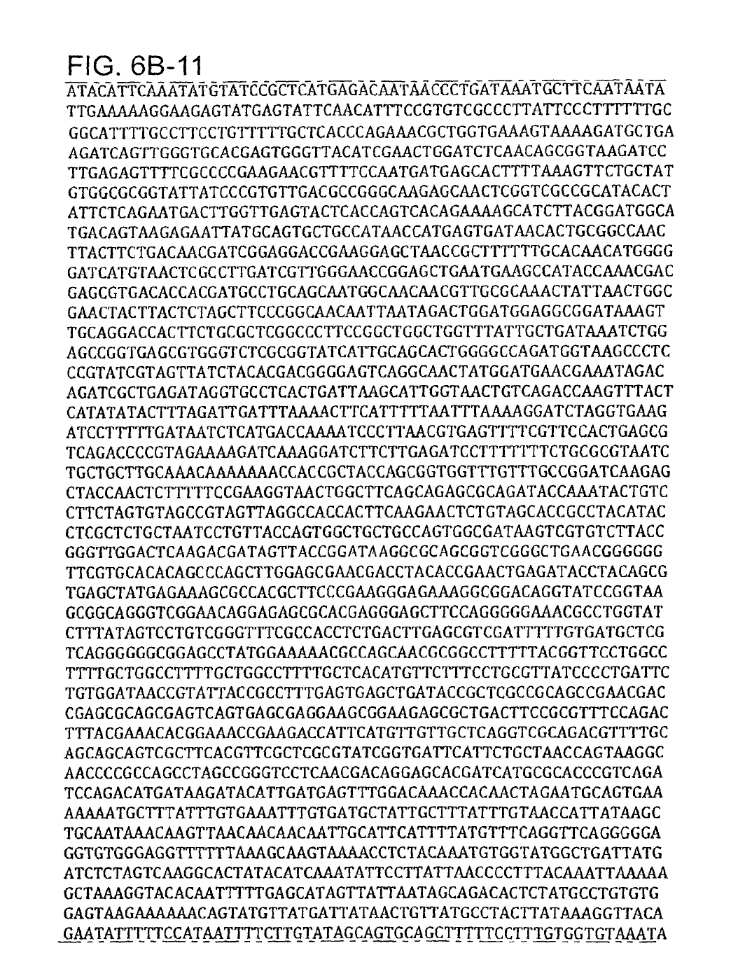

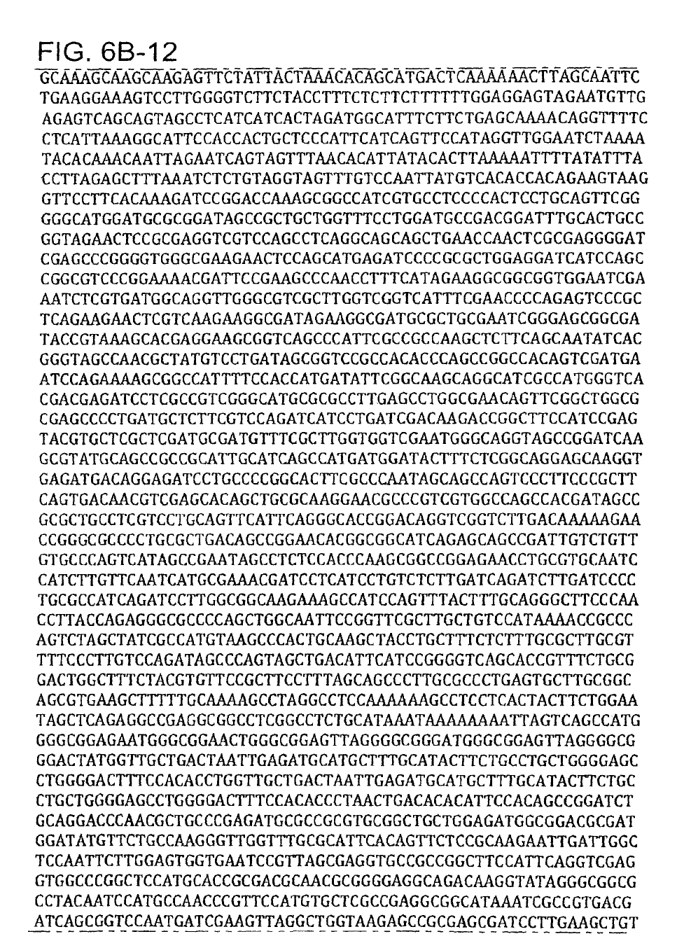

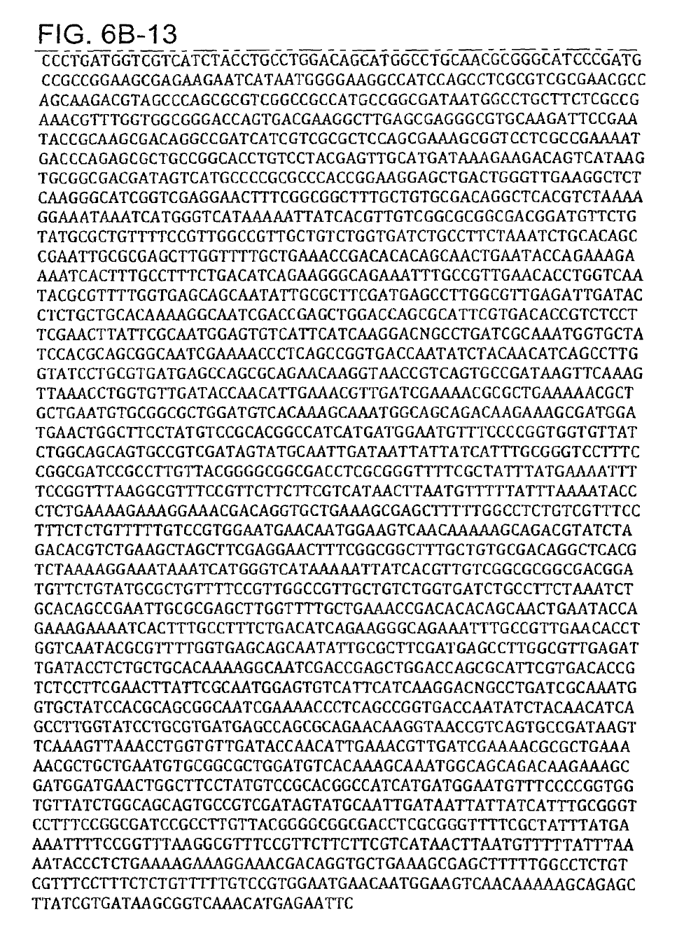

Plasmid Bc2554 (FIG. 6) contains the cetuximab heavy chain nucleotide sequence (SEQ ID NO:3) in expression vector Bc800, which includes the same chicken beta globin insulator sequence, 6.2 kb of 5′ beta casein sequence, XhoI cloning site, and 7.1 kb of 3′ beta casein flanking sequence, with the addition of a Neo^{R} marker flanked by additional chicken beta globin insulator sequences, again in a supercos backbone. The supercos vector was removed using NotI sites present at both ends.

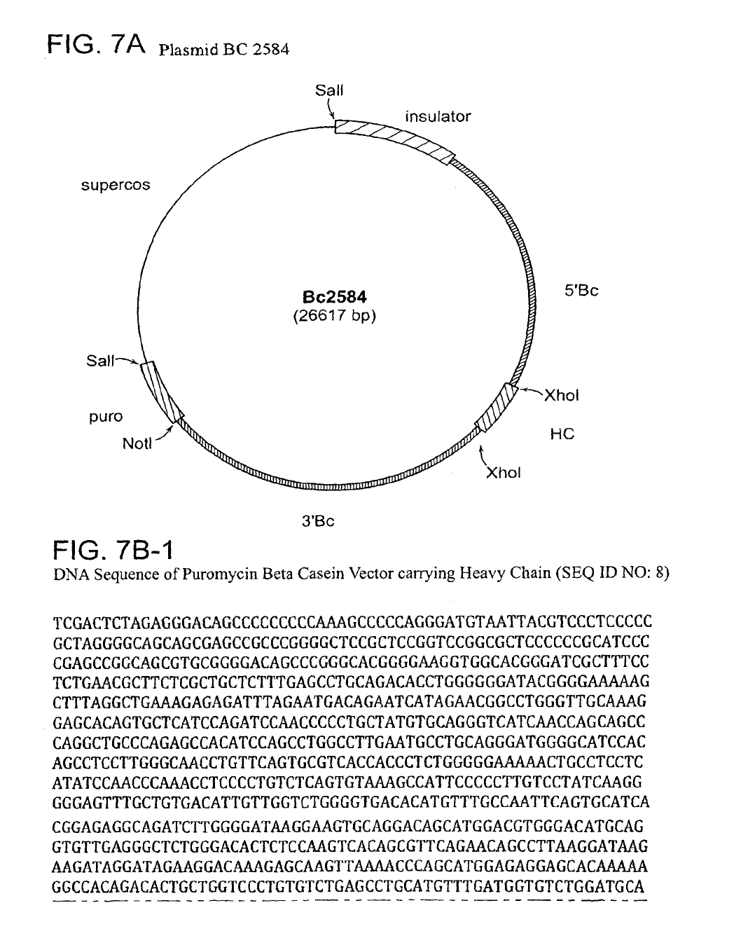

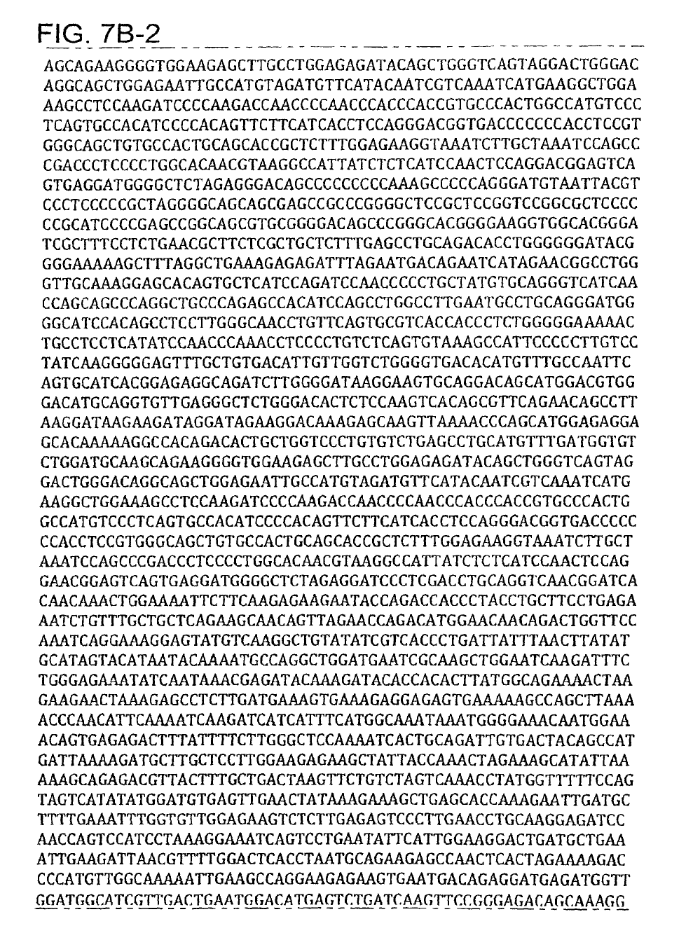

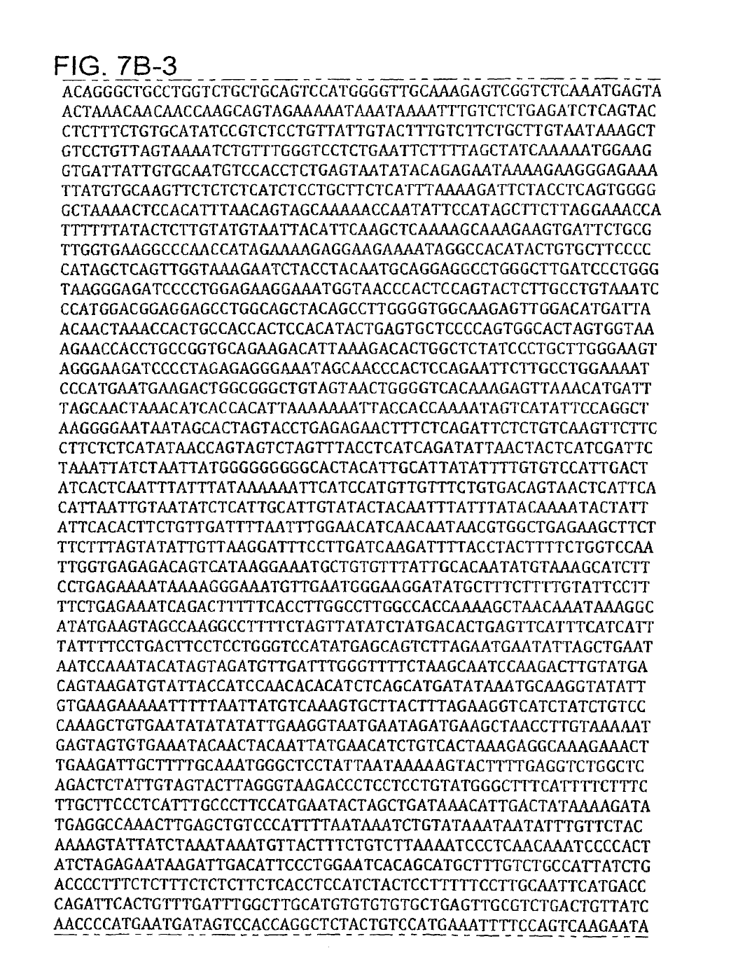

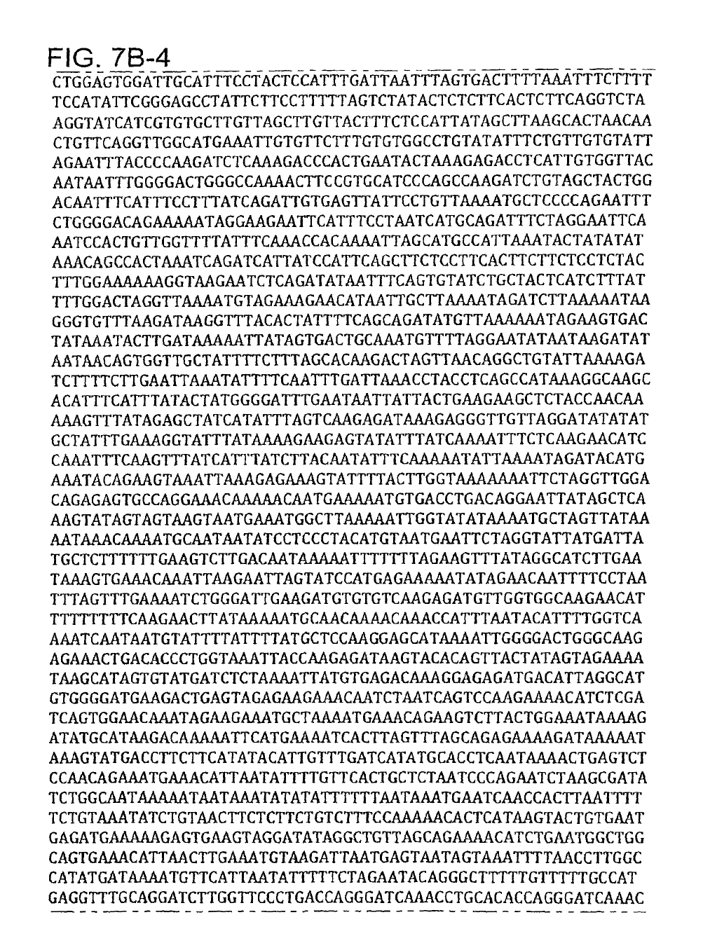

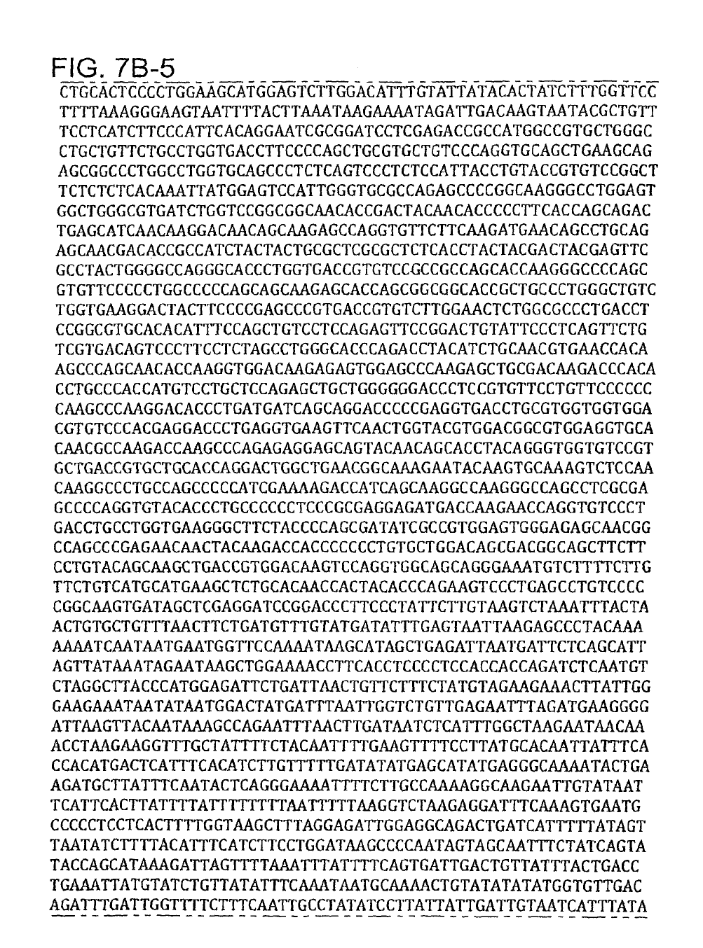

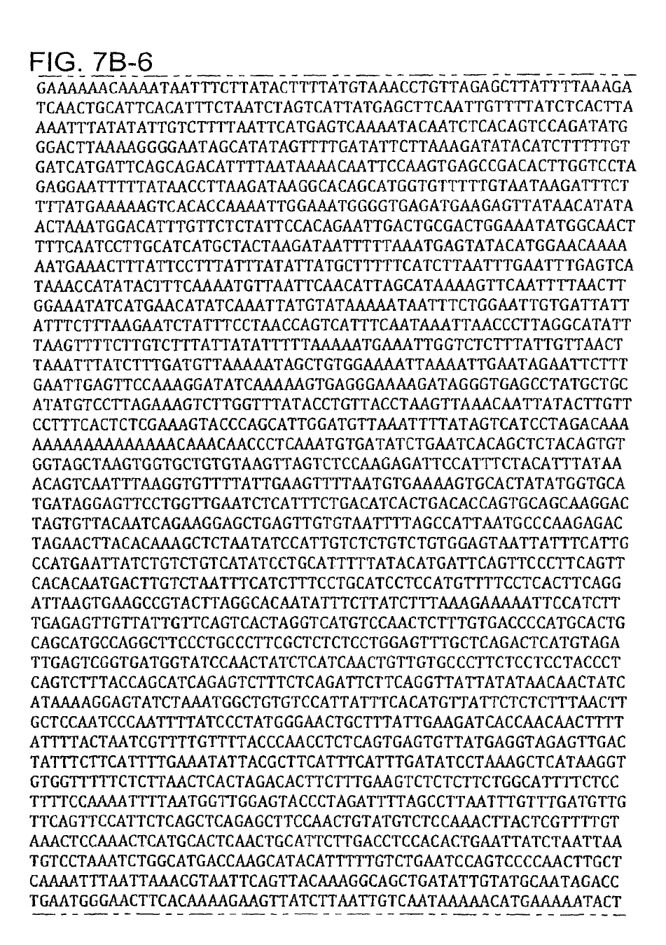

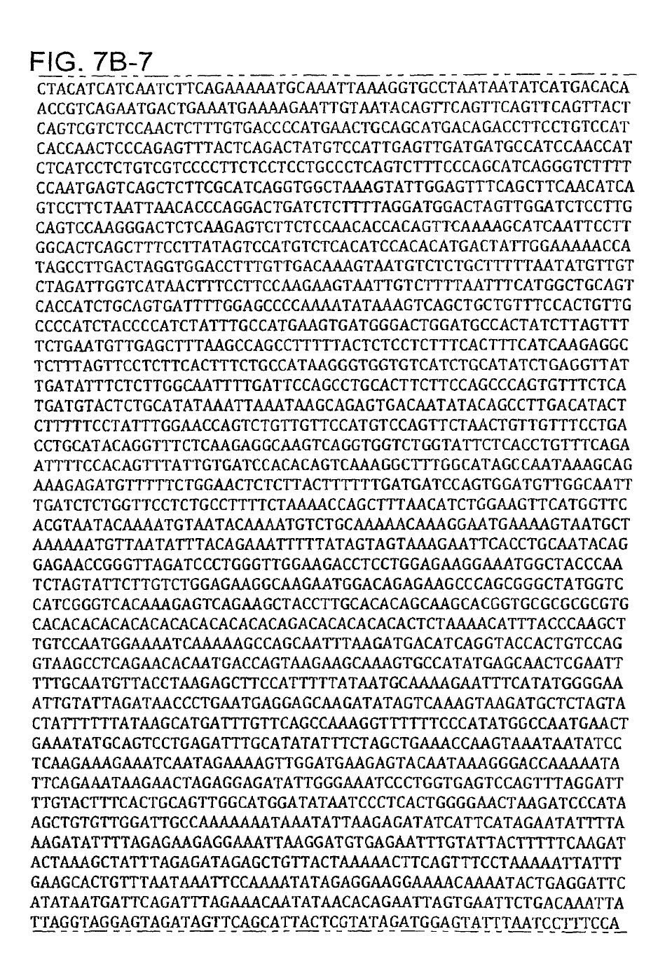

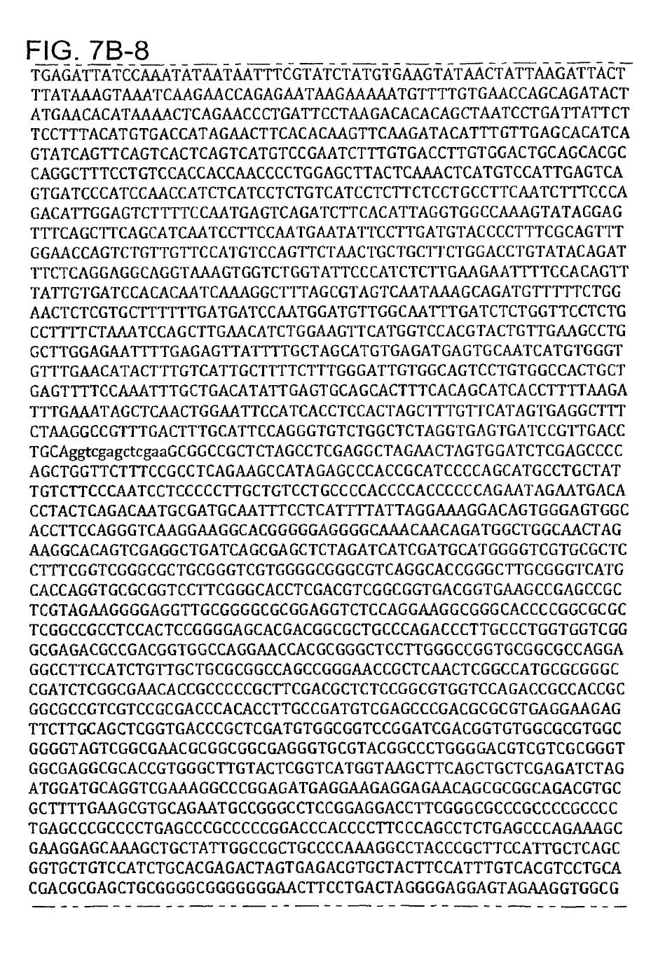

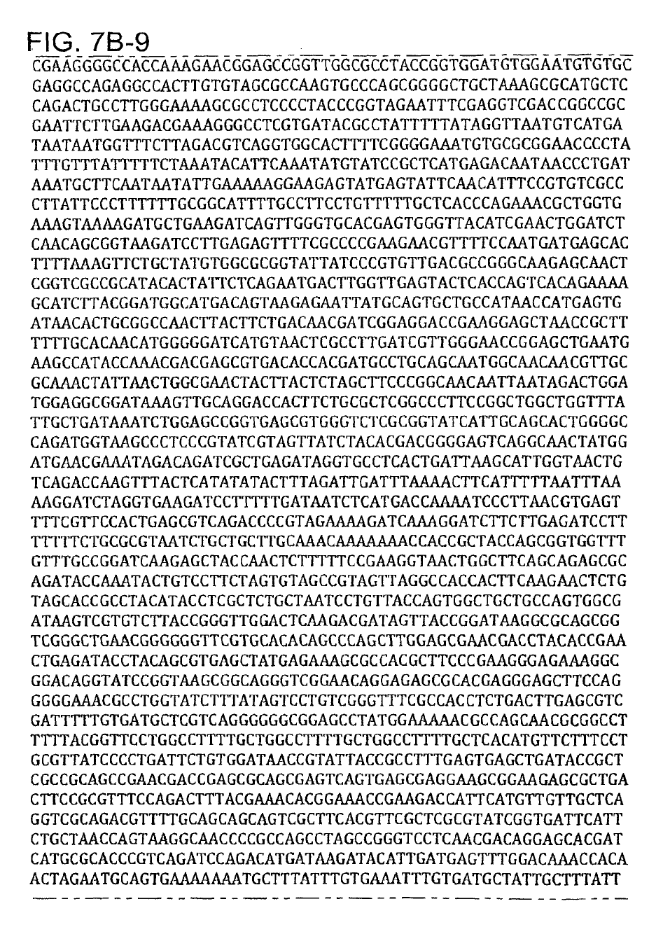

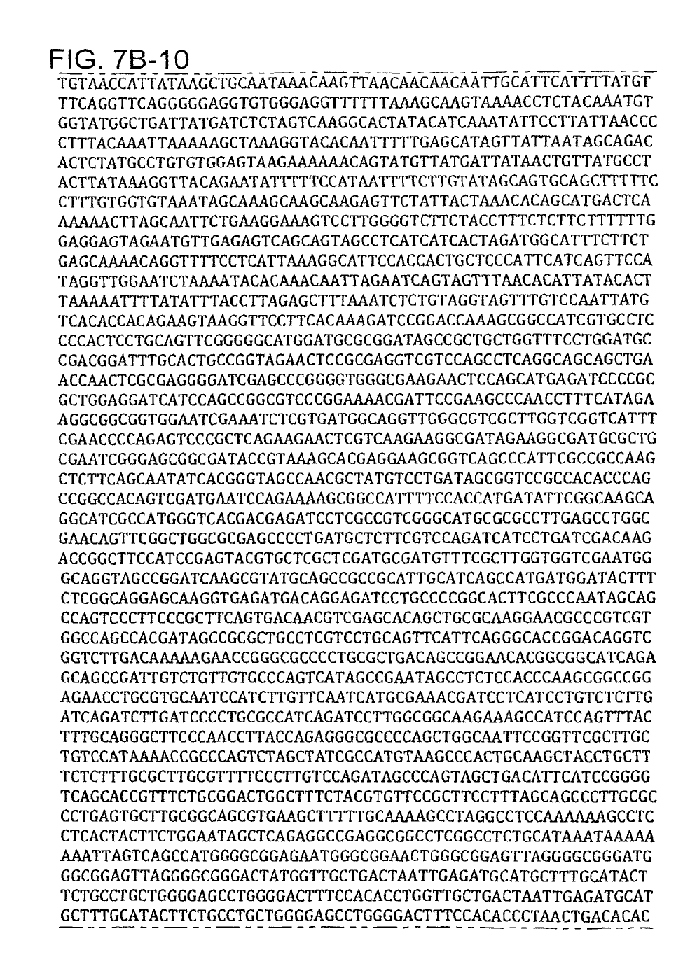

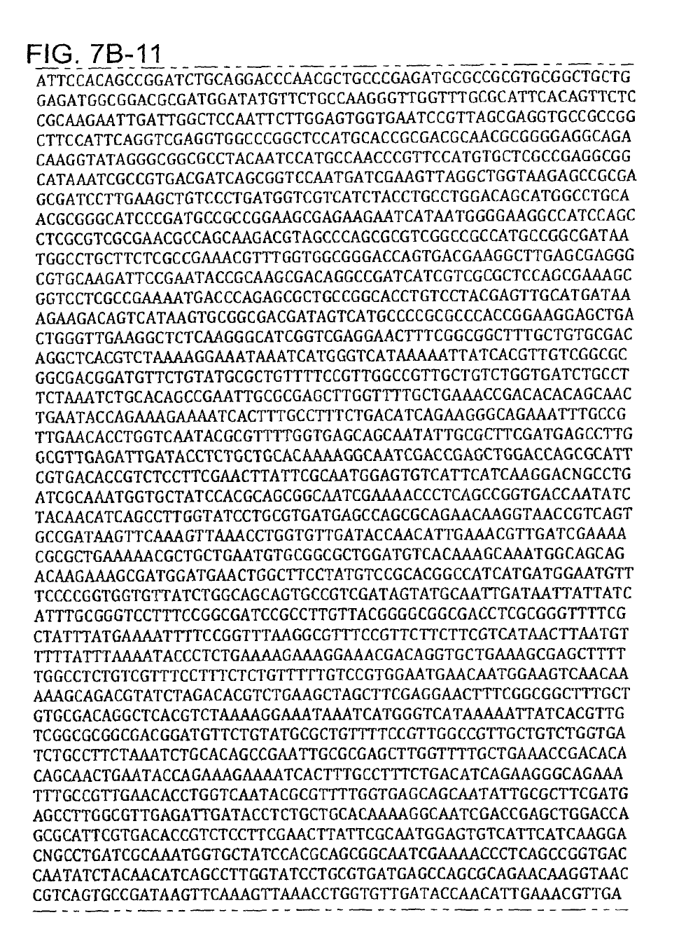

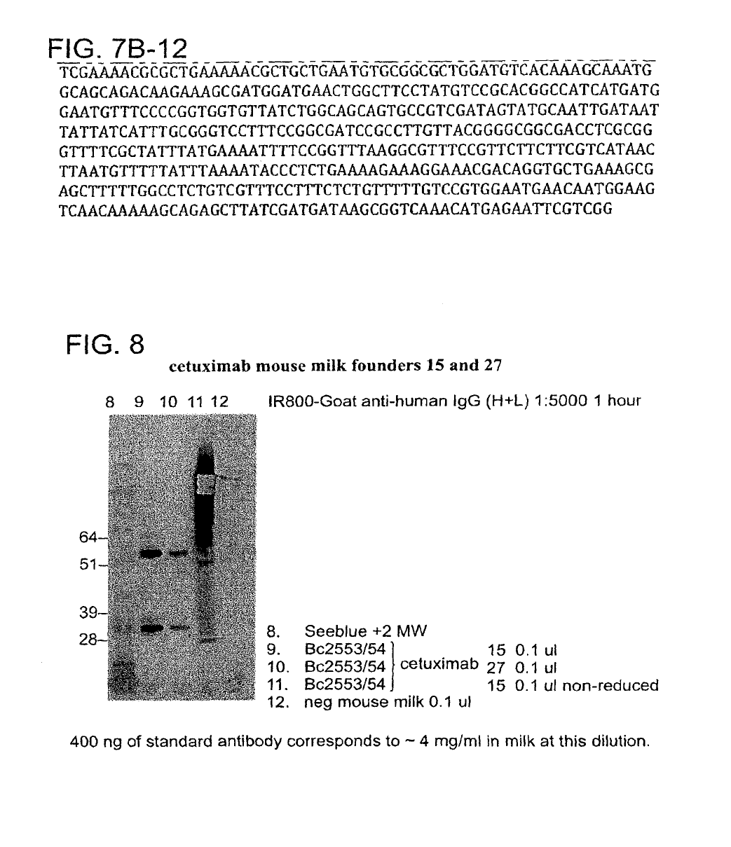

Three pieces of DNA were ligated to generate plasmid Bc2584 (FIG. 7). The first piece was made by inserting the heavy chain coding sequence into Bc350, which is identical to Bc450 described above except for the replacement of the downstream SalI site with Not I. This SalI to NotI fragment, containing the insulator, beta casein, and heavy chain sequences, was ligated to a SalI to NotI fragment of pGL71, containing the puromycin resistance gene, and a supercos fragment with flanking SalI sites.

### Generation of Transgenic Mice

The beta casein constructs carrying the heavy and light chains of cetuximab (SEQ ID NO:6 and SEQ ID NO:5; BC2554 and BC2553, respectively) were used to generate transgenic mice using standard pro-nuclear micro-injection technology. The injection fragment for the heavy chain was isolated from BC2554 HC Neo, by digesting with NotI to release the supercos backbone and electro-eluting the beta casein heavy chain injection fragment. Likewise, the supercos was removed from the beta casein light chain fragment by digesting with Sall followed by electro-elution of the injection fragment.

### Generation of Transgenic Goats

Transgenic goats were generated by Somatic Cell Nuclear Transfer, (SCNT) or cloning. A selection marker was added to the beta casein expression constructs in order to select cell cultures with the desired transgenes. The marker was added by ligating the puromycin resistance gene to beta casein heavy chain to yield BC2584 HC puro (SEQ ID NO:7; FIG. 7). The DNA constructs were used to transfect primary female fibroblasts. Following selection, the clones were characterized to ensure that both heavy and light chain constructs were integrated. The SCNT was carried out and two female goats were generated from the G100 cell line. These two goats #1 and #2 were induced to lactate in order to produce cetuximab in their milk.

### LC/MS

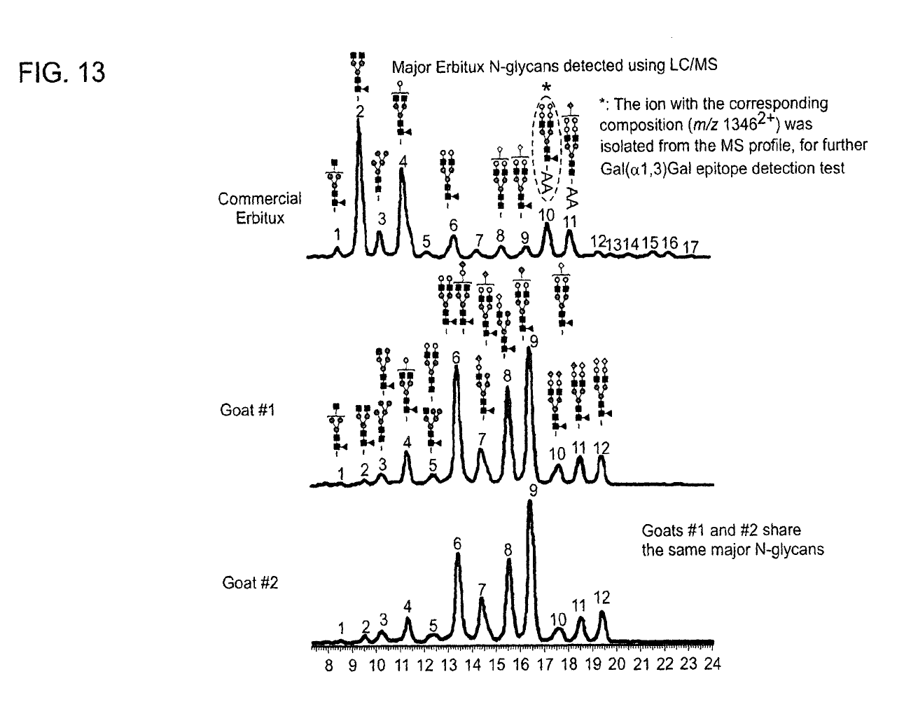

Purified cetuximab from goats #1 and #2 and commercial Erbitux® were analyzed by liquid chromatography-mass spectrometry (LC/MS) using methods well-known in the art (See FIG. 13).

### Results

### Peptide Map

The peptide map confirmed that the sequence of the variable region of commercially produced cetuximab (Erbitux®) was correct as published in Charlotte Magdelaine-Beuzelin et al., (Critical Reviews in Oncology/Hematology 64 (2007) 210-225). In addition, the peptide maps confirmed that the heavy chain constant region was human IgG1 and the light chain carried the kappa constant region.

### Expression in Mice

Several lines of mice were identified that carried both the heavy and light chain constructs of cetuximab in their genome. The mice were bred upon maturity and milked following parturition. Milk was analyzed by Western blot for the presence of cetuximab to determine level of expression. The levels of expression of cetuximab in two of the mouse lines (#15 and #27), is shown in FIG. 8.

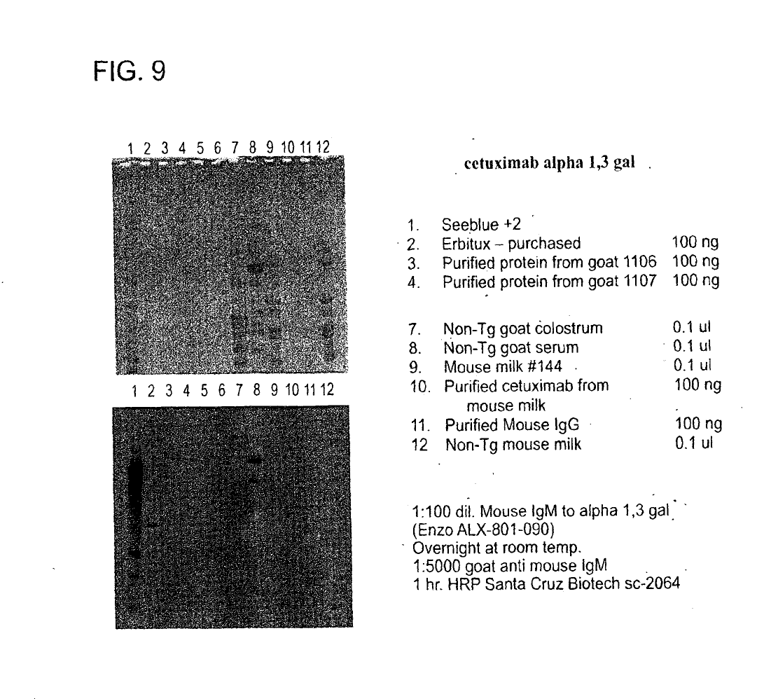

Cetuximab was purified from the milk of the mice and analyzed on a Western blot to determine if alpha-1,3-gal was present. As seen in FIG. 9, there is no evidence of alpha-1,3-gal in the mouse produced antibody (Lane 10). In contrast, commercially produced cetuximab (Erbitux®) did show alpha-1,3-gal (Lane 2).

### Expression in Goats

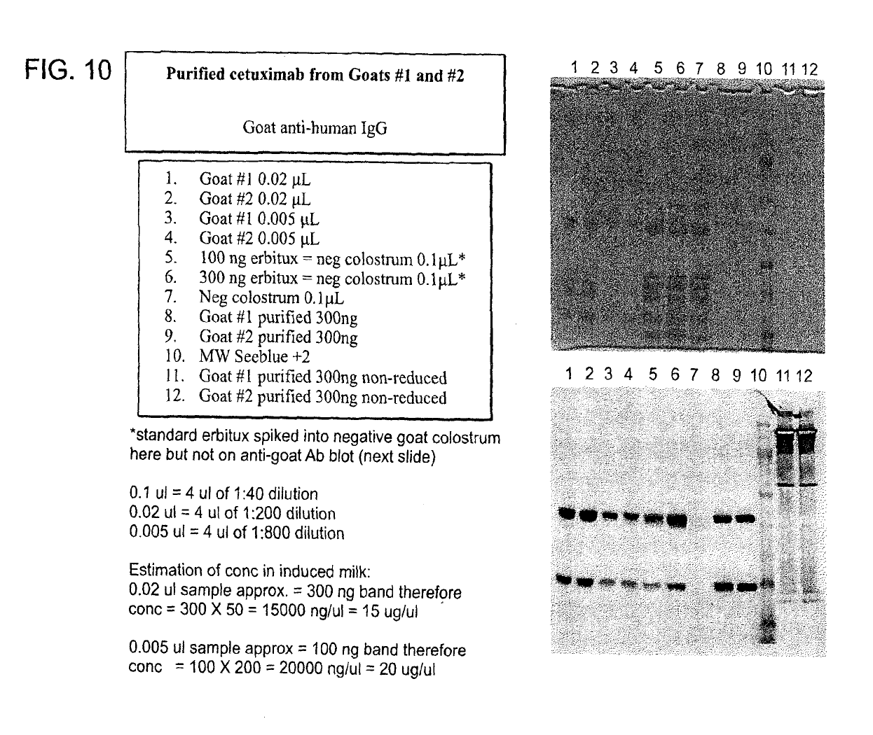

The transgenic goats that were derived from the cell line G100 using the methods described above, were transgenic for the beta casein vector linked to both heavy and light chains of cetuximab. The animals were induced to lactate at 3 months of age. Milk was collected and analyzed by Western blot to determine level of expression of cetuximab. Goats #1 and #2 each produced 10-15 mg/ml of cetuximab in their milk as shown by Western blot in which anti-human IgG1 was used to detect the recombinant antibody (FIG. 10).

### Glycosylation Analysis

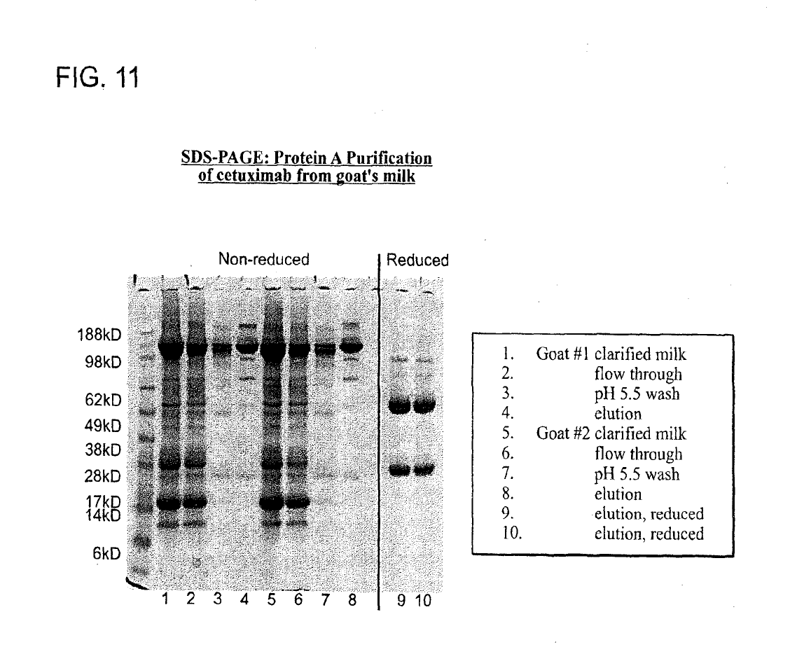

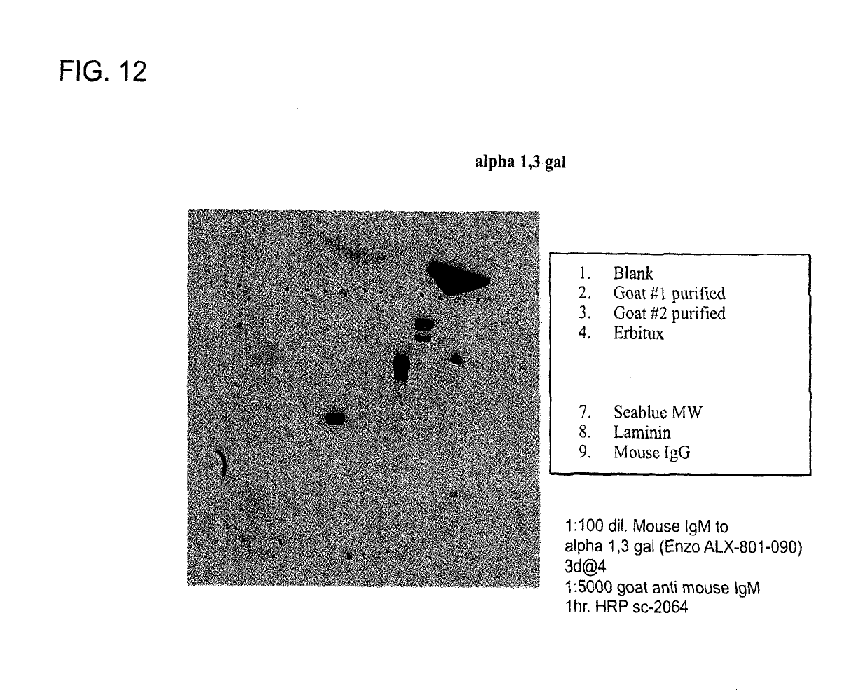

In order to determine if alpha-1,3-gal was present on the goat milk-produced cetuximab, the antibody was purified as shown in FIG. 11. A Western blot was subsequently performed using a monoclonal antibody specific to alpha-1,3-gal (FIG. 12). As a positive control, bovine laminin was used. The blot showed that commercially produced Erbitux® and laminin bound the alpha-1,3-gal monoclonal antibody, but the cetuximab produced in the milk of either goat #1 or #2 (lanes 2 and 3) did not bind the gal-alpha-1,3-gal monoclonal antibody. Thus, the goat-milk produced antibody does not include alpha-1,3-gal to the N-linked site. However, interestingly, mouse IgG (lane 9), as well as goat serum (FIG. 9; lane 8) did show gal-alpha-1,3-gal.

The purified goat milk-produced cetuximab from both goat #1 and goat #2 was also analyzed by LC/MS and compared to commercial Erbitux®. As shown in FIG. 13, alpha-1,3-gal was present on Erbitux®, but not present on the cetuximab produced in the milk of transgenic goats. Thus, the glycosylation site in the heavy chain variable region of cetuximab was not decorated with alpha-1,3-gal when produced in the mammary gland of a goat. In contrast, cetuximab produced commercially in mouse cells (i.e., Erbitux®) does show alpha-1,3 gal glycosylation. Thus, production in the mammary gland does not include alpha-1,3-gal glycosylation, even to sites normally decorated in this manner in other systems, e.g., in non-mammary mouse cells.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

What is claimed is:

1. A glycosylated antibody comprising:
(a) a heavy chain comprising SEQ ID NO:1;
(b) a light chain comprising SEQ ID NO:2;
wherein the glycosylated antibody has fewer galactose-alpha-1,3-galactose moieties than the glycosylated antibody produced in non-mammary cell culture.

2. The glycosylated antibody of claim 1, wherein the non-mammary cell culture is mouse myeloma cell culture.

3. The glycosylated antibody of claim 1 or 2, wherein the fewer galactose-alpha-1,3-galactose moieties are located in the variable region of the heavy chain.

4. The glycosylated antibody of any one of claims 1-3, wherein the fewer galactose-alpha-1,3-galactose moieties are located at position 107 of the heavy chain as shown in SEQ ID NO:1.

5. The glycosylated antibody of claim 1, wherein the glycosylated antibody lacks galactose-alpha-1,3-galactose moieties.

6. The glycosylated antibody of any one of claims 1-5, wherein the glycosylated antibody is produced in mammary gland epithelial cells.

7. The glycosylated antibody of any one of claims 1-6, wherein the glycosylated antibody is produced in the mammary gland epithelial cells of a non-human transgenic mammal.

8. The glycosylated antibody of claim 7, wherein the non-human mammal is a goat, sheep, bison, camel, cow, pig, rabbit, buffalo, horse, rat, mouse or llama.

9. The glycosylated antibody of claim 7, wherein the non-human mammal is a goat.

10. The glycosylated antibody of any one of claims 1-9, wherein the antibody is chimeric, humanized or a fully human antibody.

11. The glycosylated antibody of any one of claims 1-10, wherein the antibody is cetuximab.

12. A composition comprising the glycosylated antibody of any one of claims 1-11, and a pharmaceutically-acceptable carrier.

13. A composition comprising a population of glycosylated antibodies, wherein the glycosylated antibody comprises:
(a) a heavy chain comprising SEQ ID NO:1;
(b) a light chain comprising SEQ ID NO:2; and
wherein the population of antibodies has fewer galactose-alpha-1,3-galactose moieties than the population of glycosylated antibodies produced in non-mammary cell culture.

14. The composition of claim 13, wherein the non-mammary cell culture is mouse myeloma cell culture.

15. The composition of claim 13 or 14, wherein the fewer galactose-alpha-1,3-galactose moieties are located in the variable region of the heavy chain.

16. The composition of any one of claims 13-15, wherein the fewer galactose-alpha-1,3-galactose moieties are located at position 107 of the heavy chain as shown in SEQ ID NO:1.

17. The composition of claim 13, wherein the glycosylated antibodies lack galactose-alpha-1,3-galactose moieties.

18. The composition of any one of claims 13-17, wherein the population of glycosylated antibodies is produced in mammary gland epithelial cells.

19. The composition of any one of claims 13-18, wherein the population of glycosylated antibodies is produced in the mammary gland epithelial cells of a non-human transgenic mammal.

20. The composition of claim 19, wherein the non-human mammal is a goat, sheep, bison, camel, cow, pig, rabbit, buffalo, horse, rat, mouse or llama.

21. The composition of claim 19, wherein the non-human mammal is a goat.

22. The composition of any one of claims 13-21, wherein the glycosylated antibodies in the population are chimeric, humanized or fully human antibodies.

23. The composition of any one of claims 13-22, wherein the glycosylated antibodies in the population are cetuximab antibodies.

24. The composition of any one of claims 13-23, wherein the composition further comprises milk.

25. The composition of any one of claims 13-24, wherein the composition further comprises a pharmaceutically acceptable carrier.

26. A composition comprising monoclonal antibodies, wherein the monoclonal antibodies comprise:
(a) a heavy chain comprising SEQ ID NO:1;
(b) a light chain comprising SEQ ID NO:2; and
wherein the composition is produced in a mammary gland of a transgenic goat.

27. A composition comprising monoclonal antibodies, wherein the monoclonal antibodies comprise:
(a) a heavy chain comprising SEQ ID NO:1;
(b) a light chain comprising SEQ ID NO:2; and
wherein the monoclonal antibodies lack galactose-alpha-1,3-galactose.

28. A method comprising:
producing a population of glycosylated antibodies in mammary gland epithelial cells such that the population of glycosylated antibodies produced comprises fewer galactose-alpha-1,3-galactose moieties than the population of glycosylated antibodies produced in non-mammary cell culture, and
wherein the glycosylated antibodies comprise a heavy chain comprising SEQ ID NO:1 and a light chain comprising SEQ ID NO:2.

29. The method of claim 28, wherein the non-mammary cell culture is mouse myeloma cell culture.

30. The method of claim 28 or 29, wherein the population of glycosylated antibodies lacks galactose-alpha-1,3-galactose.

31. The method of any one of claims 28-30, wherein the method further comprises collecting the population of glycosylated antibodies.

32. The method of any one of claims 28-31, wherein the producing occurs in vitro.

33. The method of any one of claims 28-31, wherein the producing occurs in vivo.

34. The method of claim 33, wherein the producing occurs in a non-human transgenic mammal.

35. The method of claim 34, wherein the non-human mammal is a goat, sheep, bison, camel, cow, pig, rabbit, buffalo, horse, rat, mouse or llama.

36. The method of claim 34, wherein the non-human mammal is a goat.

37. The method of any one of claims 28-36, the method further comprising purifying the population of glycosylated antibodies.

38. The method of any one of claim 28-37, wherein the method further comprises
comparing the number of galactose-alpha-1,3-galactose moieties present in the population of glycosylated antibodies to the number of galactose-alpha-1,3-galactose moieties present in a population of glycosylated antibodies produced in non-mammary cell culture.

39. A population of glycosylated antibodies produced by the method of any one of claims 28-38.

40. Mammary gland epithelial cells that produce the antibody of any one of claims 1-11.

41. The mammary gland epithelial cells of claim 40, wherein the mammary gland epithelial cells comprise a nucleic acid comprising SEQ ID NO:3 and a nucleic acid comprising SEQ ID NO:4.

42. The mammary gland epithelial cells of claim 41, wherein the nucleic acid comprising SEQ ID NO:3 and the nucleic acid comprising SEQ ID NO:4 are connected.

43. A transgenic non-human mammal comprising the mammary gland epithelial cells of any one of claims 40-42.

44. Mammary gland epithelial cells that produce the population of antibodies of any one of claims 13-27.

45. The mammary gland epithelial cells of claim 44, wherein the mammary gland epithelial cells comprise a nucleic acid comprising SEQ ID NO: 3 and a nucleic acid comprising SEQ ID NO: 4.

46. The mammary gland epithelial cells of claim 45, wherein the nucleic acid comprising SEQ ID NO: 3 and the nucleic acid comprising SEQ ID NO: 4 are connected.

47. A transgenic non-human mammal comprising the mammary gland epithelial cells of any one of claims 44-46.

48. The transgenic non-human mammal of claim 43 or 47, wherein the transgenic non-human mammal is a goat.

49. A method comprising:
administering an effective amount of the antibody of any one of claims 1-11 or the composition of any one of claims 12-27 to a subject in need thereof.

50. The method of claim 49, wherein the subject has cancer.

51. The method of claim 50, wherein the cancer is head and neck cancer or colorectal cancer.

52. The method of any one of claims 49-51, wherein the method further comprises administering at least one additional therapeutic agent.

53. The method of claim 52, wherein the at least one additional therapeutic agent is irinotecan, leucovorin calcium, fluorouracil, or a combination thereof.
